(19) United States
(12) Patent Application Publication
(10) Pub. No.: US 2025/0099447 A1
(43) Pub. Date: Mar. 27, 2025

(54) SOLID FORMS OF FXR AGONISTS

(71) Applicant: Gilead Sciences, Inc., Foster City, CA (US)

(72) Inventors: Derek M. Dalton, Berkeley, CA (US); Peter C. Fung, San Mateo, CA (US); Nolan Griggs, San Mateo, CA (US); Jeffrey N. Hemenway, San Mateo, CA (US); Olga V. Lapina, Newark, CA (US); Matthew M. Logan, San Mateo, CA (US); Sean T. Neville, San Mateo, CA (US); Bryan J. Reynolds, Chicago, IL (US); Hui-Wen Shih, San Mateo, CA (US); Anna M. Wagner, San Francisco, CA (US)

(21) Appl. No.: 18/818,487

(22) Filed: Aug. 28, 2024

Related U.S. Application Data
(62) Division of application No. 18/051,471, filed on Oct. 31, 2022, now Pat. No. 12,102,625.
Application No. 18/051,471 is a division of application No. 16/791,974, filed on Feb. 14, 2020, now Pat. No. 11,524,005.
(60) Provisional application No. 62/807,542, filed on Feb. 19, 2019.

(51) Int. Cl. A61K 31/4439; C07D 401/14; C07D 413/14

(52) U.S. Cl. CPC A61K 31/4439; C07D 401/14; C07D 413/14

(57) ABSTRACT

Disclosed herein are novel solid forms of FXR agonists. The disclosure also relates to pharmaceutical compositions containing one or more of the solid forms disclosed herein, as well as methods of using the solid forms in the treatment of conditions mediated by FXR. The disclosure also relates to methods for obtaining such solid forms.

### CROSS-REFERENCE TO RELATED APPLICATIONS

This application is a divisional of U.S. patent application Ser. No. 18/051,471, filed Oct. 31, 2022, which is a divisional of U.S. patent application Ser. No. 16/791,974, filed Feb. 14, 2020, now U.S. Pat. No. 11,524,005, which application claims the benefit of priority to U.S. Provisional Patent Application No. 62/807,542, filed Feb. 19, 2019, the contents of all of which are incorporated herein by reference in their entireties.

### FIELD

The present disclosure relates to novel solid forms of FXR agonists, and the pharmaceutical formulations and therapeutic uses thereof.

### BACKGROUND

The present disclosure relates to solid forms of compounds that bind to the NR1H4 receptor (FXR) and act as agonists or modulators of FXR. The disclosure further relates to the use of the solid forms of such compounds for the treatment and/or prophylaxis of diseases and/or conditions through binding of said nuclear receptor by said compounds.

Compounds that bind to the NR1H4 receptor (FXR) can act as agonists or modulators of FXR. FXR agonists are useful for the treatment and/or prophylaxis of diseases and conditions through binding of the NR1H4 receptor. One such FXR agonist is the compound of Formula I:

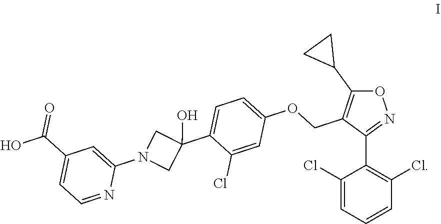 [SMILES: O=C(O)c1ccnc(N2CC(O)(c3ccc(OCc4c(-c5c(Cl)cccc5Cl)noc4C4CC4)cc3Cl)C2)c1]

Although numerous FXR agonists are known, what is desired in the art are physically stable forms of the compound of Formula I, or pharmaceutically acceptable salt thereof, with desired properties such as good physical and chemical stability, good aqueous solubility and good bioavailability. For example, pharmaceutical compositions are desired that address challenges of stability, variable pharmacodynamics responses, drug-drug interactions, pH effect, food effects, and oral bioavailability.

Accordingly, there is a need for stable forms of the compound of Formula I with suitable chemical and physical stability for the formulation, therapeutic use, manufacturing, and storage of the compound.

Moreover, it is desirable to develop a solid form of Formula I that may be useful in the synthesis of Formula I. A solid form, such as a crystalline form of a compound of Formula I may be an intermediate to the synthesis of Formula I. A solid form may have properties such as bioavailability, stability, purity, and/or manufacturability at certain conditions that may be suitable for medical or pharmaceutical uses.

### SUMMARY

In some embodiments, the present disclosure is directed to novel solid forms of a compound of Formula I:

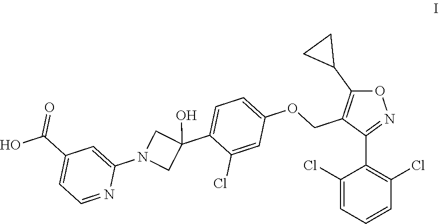 [SMILES: O=C(O)c1ccnc(N2CC(O)(c3ccc(OCc4c(-c5c(Cl)cccc5Cl)noc4C4CC4)cc3Cl)C2)c1]

and pharmaceutically acceptable salts, solvates and hydrates thereof.

These novel forms are useful, for example, for treating human patients suffering from a liver disease, such as nonalcoholic steatohepatitis (“NASH”). The novel solid forms of the present disclosure can be useful for preparing a medicament for treating a liver disease. The novel solid forms of the present disclosure can be used as FXR agonists.

In some embodiments, the present disclosure is directed to solid forms of a zwitterionic form of Formula I

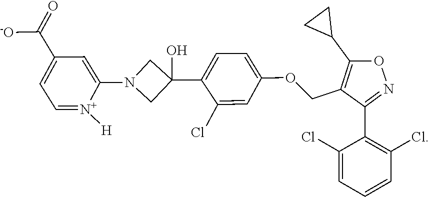

In some embodiments, the present disclosure is directed to zwitterion Form I.

In some embodiments, the present disclosure is directed to zwitterion Form II.

In some embodiments, the present disclosure is directed to zwitterion hydrate.

In some embodiments, the present disclosure is directed to zwitterion amorphous form.

In some embodiments, the present disclosure is directed to solid forms of a tromethamine salt of Formula I. In some embodiments, the tromethamine salt of Formula I has the structure:

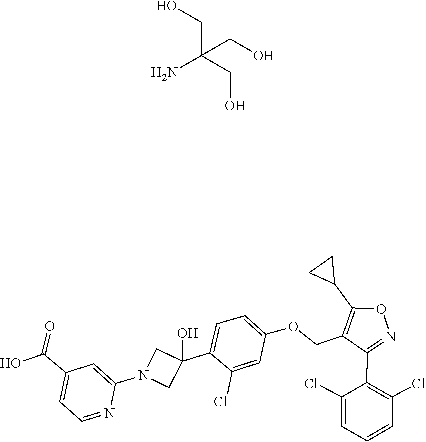 [SMILES: NC(CO)(CO)CO ; O=C(O)c1ccnc(N2CC(O)(c3ccc(OCc4c(-c5c(Cl)cccc5Cl)noc4C4CC4)cc3Cl)C2)c1]

In some embodiments, the present disclosure is directed to tromethamine salt Form I.

In some embodiments, the present disclosure is directed to tromethamine salt Form II.

In some embodiments, the present disclosure is directed to tromethamine salt hydrate I.

In some embodiments, the present disclosure is directed to tromethamine salt hydrate II.

In some embodiments, the present disclosure is directed to tromethamine salt hydrate III.

In some embodiments, the present disclosure is directed to tromethamine salt hydrate IV.

In some embodiments, the present disclosure is directed to tromethamine salt methanol solvate I.

In some embodiments, the present disclosure is directed to tromethamine salt methanol solvate II.

In some embodiments, the present disclosure is directed to tromethamine salt methanol solvate III.

In some embodiments, the present disclosure is directed to tromethamine salt methyl t-butyl ether (“MTBE”) solvate.

In some embodiments, the present disclosure is directed to tromethamine salt amorphous form.

In some embodiments, the present disclosure is directed to tromethamine salt ethanol solvate.

In some embodiments, the present disclosure is directed to solid forms of a p-toluenesulfonic acid salt of Formula I.

In some embodiments, the present disclosure is directed to p-toluenesulfonic acid salt Form I.

In some embodiments, the present disclosure is directed to p-toluenesulfonic acid salt Form II.

In some embodiments, the present disclosure is directed to p-toluenesulfonic acid salt Form III.

In some embodiments, the present disclosure is directed to p-toluenesulfonic acid salt hydrate.

In some embodiments, a pharmaceutical composition comprises a therapeutically effective amount of a solid form of Formula I.

### DESCRIPTION OF THE FIGURES

FIG. 1 shows an XRPD pattern of the zwitterionic compound of Formula I Form I.

FIG. 2 shows a DSC thermogram of the zwitterionic compound of Formula I Form I.

FIG. 3 shows a TGA thermogram of the zwitterionic compound of Formula I Form I.

FIG. 4 shows an XRPD pattern of the zwitterionic compound of Formula I Form II.

FIG. 5 shows a DSC thermogram of the zwitterionic compound of Formula I Form II.

FIG. 6 shows a TGA thermogram of the zwitterionic compound of Formula I Form II.

FIG. 7 shows an XRPD pattern of the zwitterionic compound of Formula I hydrate.

FIG. 8 shows a DSC thermogram of the zwitterionic compound of Formula I hydrate.

FIG. 9 shows a TGA thermogram of the zwitterionic compound of Formula I hydrate.

FIG. 10 shows an XRPD pattern of the amorphous zwitterionic compound of Formula I.

FIG. 11 shows an XRPD pattern of Formula I tromethamine salt Form I.

FIG. 12 shows a DSC thermogram of the Formula I tromethamine salt Form I.

FIG. 13 shows a TGA thermogram of the Formula I tromethamine salt Form I.

FIG. 14 shows an XRPD pattern of Formula I tromethamine salt Form II.

FIG. 15 shows an XRPD pattern of the Formula I tromethamine salt hydrate I.

FIG. 16 shows a DSC thermogram of the Formula I tromethamine salt hydrate I.

FIG. 17 shows a TGA thermogram of the Formula I tromethamine salt hydrate I.

FIG. 18 shows an XRPD pattern of the Formula I tromethamine salt hydrate II.

FIG. 19 shows a DSC thermogram of the Formula I tromethamine salt hydrate II.

FIG. 20 shows a TGA thermogram of the Formula I tromethamine salt hydrate II.

FIG. 21 shows an XRPD pattern of the Formula I tromethamine salt hydrate III.

FIG. 22 shows a DSC thermogram of the Formula I tromethamine salt hydrate III.

FIG. 23 shows a TGA thermogram of the Formula I tromethamine salt hydrate III.

FIG. 24 shows an XRPD pattern of the Formula I tromethamine salt hydrate IV.

FIG. 25 shows a DSC thermogram of the Formula I tromethamine salt hydrate IV.

FIG. 26 shows a TGA thermogram of the Formula I tromethamine salt hydrate IV.

FIG. 27 shows an XRPD pattern of the Formula I tromethamine salt methanol solvate I.

FIG. 28 shows an XRPD pattern of the Formula I tromethamine salt methanol solvate II.

FIG. 29 shows an XRPD pattern of the Formula I tromethamine salt methanol solvate III.

FIG. 30 shows a DSC thermogram of the Formula I tromethamine salt methanol solvate III.

FIG. 31 shows a TGA thermogram of the Formula I tromethamine salt methanol solvate III.

FIG. 32 shows an XRPD pattern of the Formula I tromethamine salt MTBE solvate.

FIG. 33 shows a DSC thermogram of the Formula I tromethamine salt MTBE solvate.

FIG. 34 shows a TGA thermogram of the Formula I tromethamine salt MTBE solvate.

FIG. 35 shows an XRPD pattern of the Formula I tromethamine salt amorphous form.

FIG. 36 shows an XRPD pattern of the Formula I tromethamine salt ethanol solvate.

FIG. 37 shows an XRPD pattern of the Formula I p-toluenesulfonic acid salt Form I.

FIG. 38 shows a DSC thermogram of the Formula I p-toluenesulfonic acid salt Form I.

FIG. 39 shows a TGA thermogram of the Formula I p-toluenesulfonic acid salt Form I.

FIG. 40 shows an XRPD pattern of the Formula I p-toluenesulfonic acid salt Form II.

FIG. 41 shows a DSC thermogram of the Formula I p-toluenesulfonic acid salt Form II.

FIG. 42 shows a TGA thermogram of the Formula I p-toluenesulfonic acid salt Form II.

FIG. 43 shows an XRPD pattern of the Formula I p-toluenesulfonic acid salt Form III.

FIG. 44 shows a DSC thermogram of the Formula I p-toluenesulfonic acid salt Form III.

FIG. 45 shows a TGA thermogram of the Formula I p-toluenesulfonic acid salt Form III.

FIG. 46 shows an XRPD pattern of the Formula I p-toluenesulfonic acid salt hydrate.

FIG. 47 shows a DSC thermogram of the Formula I p-toluenesulfonic acid salt hydrate.

FIG. 48 shows a TGA thermogram of the Formula I p-toluenesulfonic acid salt hydrate.

### DETAILED DESCRIPTION

In the following description, certain specific details are set forth in order to provide a thorough understanding of various embodiments of the disclosure. However, one skilled in the art will understand that the disclosure may be practiced without these details. The description below of several embodiments is made with the understanding that the present disclosure is to be considered as an exemplification of the claimed subject matter, and is not intended to limit the appended claims to the specific embodiments illustrated. The headings used throughout this disclosure are provided for convenience only and are not to be construed to limit the claims in any way. Embodiments illustrated under any heading may be combined with embodiments illustrated under any other heading.

### Definitions

Unless the context requires otherwise, throughout the present specification and claims, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Embodiments that reference throughout this specification to “a compound” include the crystalline, salt, co-crystal, zwitterion, hydrate, solvate, and/or amorphous forms of the formulas and/or compounds disclosed herein. Thus, the appearance or the phrase “a compound of Formula I” can include Formula I zwitterion Form I; Formula I zwitterion Form II; Formula I hydrate; Formula I zwitterion amorphous; Formula I tromethamine salt Form I; Formula I tromethamine salt Form II; Formula I tromethamine salt hydrate I; Formula I tromethamine salt hydrate II; Formula I tromethamine salt hydrate III; Formula I tromethamine salt hydrate IV; Formula I tromethamine salt methanol solvate I; Formula I tromethamine salt methanol solvate II; Formula I tromethamine salt methanol solvate III; Formula I tromethamine salt MTBE solvate; Formula I tromethamine salt amorphous form; Formula I tromethamine salt ethanol solvate; Formula I p-toluenesulfonic acid salt Form I; Formula I p-toluenesulfonic acid salt Form II; Formula I p-toluenesulfonic acid salt Form III; and/or Formula I p-toluenesulfonic acid salt hydrate.

The disclosure disclosed herein is also meant to encompass all pharmaceutically acceptable compounds of Formula I being isotopically-labeled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabeled compounds could be useful to help determine or measure the effectiveness of the compounds, by characterizing, for example, the site or mode of action, or binding affinity to pharmacologically important site of action. Certain isotopically labeled compounds of Formula I, for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability. For example, in vivo half-life may increase or dosage requirements may be reduced. Thus, heavier isotopes may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically labeled compounds of Formula I can generally be prepared by conventional techniques known to those skilled in the art or by processes analogous to those described in the Examples as set out below using an appropriate isotopically labeled reagent in place of the non-labeled reagent previously employed.

“Stable compound” and “stable structure” are meant to indicate a compound that is sufficiently robust to survive isolation to a useful degree of purity from a reaction mixture, and formulation into an efficacious therapeutic agent.

“Optional” or “optionally” means that the subsequently described event or circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, and/or emulsifier, or a combination of one or more of the above which has been approved by the United States Food and Drug Administration as being acceptable for use in humans or domestic animals.

A “pharmaceutical composition” refers to a formulation of a compound of the disclosure (e.g., a compound of Formula I) and a medium generally accepted in the art for the delivery of the biologically active compound to mammals, e.g., humans. Such a medium includes all pharmaceutically acceptable excipients therefor.

“Effective amount” or “therapeutically effective amount” refers to an amount of a compound according to the disclosure, which when administered to a patient in need thereof, is sufficient to effect treatment for disease-states, conditions, or disorders for which the compounds have utility. Such an amount would be sufficient to elicit the biological or medical response of a tissue system, or patient that is sought by a researcher or clinician. The amount of a compound according to the disclosure which constitutes a therapeutically effective amount will vary depending on such factors as the compound and its biological activity, the composition used for administration, the time of administration, the route of administration, the rate of excretion of the compound, the duration of the treatment, the type of disease-state or disorder being treated and its severity, drugs used in combination with or coincidentally with the compounds of the disclosure, and the age, body weight, general health, sex and diet of the patient. Such a therapeutically effective amount can be determined routinely by one of ordinary skill in the art having regard to their own knowledge, the state of the art, and this disclosure.

“Prevention” or “preventing” or “prophylaxis” means any treatment of a disease or condition that causes the clinical symptoms of the disease or condition not to develop. Compounds may, in some embodiments, be administered to a subject (including a human) who is at risk or has a family history of the disease or condition.

“Treating” and “treatment” of a disease include the following:
(1) preventing or reducing the risk of developing the disease, i.e., causing the clinical symptoms of the disease not to develop in a subject that may be exposed to or predisposed to the disease but does not yet experience or display symptoms of the disease,
(2) inhibiting the disease, i.e., arresting or reducing the development of the disease or its clinical symptoms, and
(3) relieving the disease, i.e., causing regression of the disease or its clinical symptoms.

The terms “subject” or “patient” refer to an animal, such as a mammal (including a human), that has been or will be the object of treatment, observation or experiment. The methods described herein may be useful in human therapy and/or veterinary applications. In some embodiments, the subject is a mammal (or the patient). In some embodiments the subject (or the patient) is human, domestic animals (e.g., dogs and cats), farm animals (e.g., cattle, horses, sheep, goats, and pigs), and/or laboratory animals (e.g., mice, rats, hamsters, guinea pigs, pigs, rabbits, dogs, and monkeys). In some embodiments, the subject (or the patient) is a human. “Human (or patient) in need thereof” refers to a human who may have or is suspect to have diseases or conditions that would benefit from certain treatment; for example, being treated with the compounds disclosed herein according to the present application.

Reference to “about” a value or parameter herein includes (and describes) embodiments that are directed to that value or parameter per se. For example, description referring to “about X” includes description of “X.” Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Pharmaceutically acceptable” or “physiologically acceptable” refer to compounds, salts, compositions, dosage forms and other materials which are useful in preparing a pharmaceutical composition that is suitable for veterinary or human pharmaceutical use.

“Unit dosage forms” are physically discrete units suitable as unitary dosages for subjects (e.g., human subjects and other mammals), each unit containing a predetermined quantity of active material calculated to produce the desired therapeutic effect, in association with a suitable pharmaceutical excipient.

The term “substantially as shown in” when referring, for example, to an XRPD pattern, a DSC thermogram, a DVS isotherm, or a TGA thermogram includes a pattern, thermogram or spectrum that is not necessarily identical to those depicted herein, but that falls within the limits of experimental error or deviations when considered by one of ordinary skill in the art.

In some embodiments, the term “substantially pure” or “substantially free” with respect to a particular crystalline form of a compound means that the composition comprising the crystalline form contains less than 99%, less than 95%, less than 90%, less than 85%, less than 80%, less than 75%, less than 70%, less than 65%, less than 60%, less than 55%, less than 50%, less than 40%, less than 30%, less than 20%, less than 15%, less than 10%, less than 5%, or less than 1% by weight of other substances, including other crystalline forms and/or impurities. In certain embodiments, “substantially pure” or “substantially free of” refers to a substance free of other substances, including other crystalline forms and/or impurities. Impurities may, for example, include by-products or left-over reagents from chemical reactions, contaminants, degradation products, other crystalline forms, water, and solvents.

The term “pharmaceutically acceptable salts” refers to salts prepared from pharmaceutically acceptable non-toxic bases or acids, including inorganic bases or acids and organic bases or acids. In case the compounds of the present disclosure contain one or more acidic or basic groups, the disclosure also comprises their corresponding pharmaceutically or toxicologically acceptable salts, in particular their pharmaceutically utilizable salts. Thus, the compounds of the present disclosure which contain acidic groups can be present on these groups and can be used according to the disclosure, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine or amino acids. The compounds of the present disclosure which contain one or more basic groups, i.e. groups which can be protonated, can be present and can be used according to the disclosure in the form of their addition salts with inorganic or organic acids. Examples of suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the compounds of the present disclosure simultaneously contain acidic and basic groups in the molecule, the disclosure also includes, in addition to the salt forms mentioned, inner salts or betaines (zwitterions). The respective salts can be obtained by customary methods which are known to the person skilled in the art like, for example, by contacting these with an organic or inorganic acid or base in a solvent or dispersant, or by anion exchange or cation exchange with other salts. The present disclosure also includes all salts of the compounds of the present disclosure which, owing to low physiological compatibility, are not directly suitable for use in pharmaceuticals but which can be used, for example, as intermediates for chemical reactions or for the preparation of pharmaceutically acceptable salts.

Further the compounds of the present disclosure may be present in the form of solvates, such as those which include as solvate water, or pharmaceutically acceptable solvates, such as alcohols, in particular ethanol. A “solvate” is formed by the interaction of a solvent and a compound. When the solvent is water, the “solvate” is a “hydrate.”

In certain embodiments, provided are optical isomers, racemates, or other mixtures thereof of the compounds described herein or a pharmaceutically acceptable salt or a mixture thereof. If desired, isomers can be separated by methods well known in the art, e.g. by liquid chromatography. In those situations, the single enantiomer or diastereomer, i.e., optically active form, can be obtained by asymmetric synthesis or by resolution. Resolution can be accomplished, for example, by conventional methods such as crystallization in the presence of a resolving agent, or chromatography, using for example, a chiral high-pressure liquid chromatography (HPLC) column.

A “stereoisomer” refers to a compound made up of the same atoms bonded by the same bonds but having different three-dimensional structures, which are not interchangeable. The present invention contemplates various stereoisomers and mixtures thereof and includes “enantiomers,” which refers to two stereoisomers whose molecules are nonsuperimposeable mirror images of one another. “Diastereomers” are stereoisomers that have at least two asymmetric atoms, but which are not mirror-images of each other.

The compounds disclosed herein and their pharmaceutically acceptable salts may include an asymmetric center and may thus give rise to enantiomers, diastereomers, and other stereoisomeric forms that may be defined, in terms of absolute stereochemistry, as (R)- or(S) or, as (D)- or (L)- for amino acids. The present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high-pressure liquid chromatography (HPLC). When the compounds described herein contain olefinic double bonds or other centres of geometric asymmetry, and unless specified otherwise, it is intended that the compounds include both E and Z geometric isomers.

Compositions provided herein that include a compound described herein or pharmaceutically acceptable salts, isomer, or a mixture thereof may include racemic mixtures, or mixtures containing an enantiomeric excess of one enantiomer or single diastereomers or diastereomeric mixtures. All such isomeric forms of these compounds are expressly included herein the same as if each and every isomeric form were specifically and individually listed.

A “tautomer” is an isomer of a compound which exists together, in equilibrium, with another isomer where the two isomers are interchangeable by migration of an atom or a group within the molecule.

### Solid Forms of Formula I

Solid forms of Formula I, including crystalline forms and substantially pure forms, may provide the advantage of bioavailability and stability, suitable for use as an active ingredient in a pharmaceutical composition. Formula I tromethamine salt Form I, for example, exhibits advantageous physical properties such as good physical and chemical stability, good aqueous solubility and good bioavailability. Variations in the crystal structure of a pharmaceutical drug substance or active ingredient may affect the dissolution rate (which may affect bioavailability, etc.), manufacturability (e.g., ease of handling, ability to consistently prepare doses of known strength), and stability (e.g., thermal stability, shelf life, etc.) of a pharmaceutical drug product or active ingredient. Such variations may affect the preparation or formulation of pharmaceutical compositions in different dosage or delivery forms, such as solutions or solid oral dosage form including tablets and capsules. Compared to other forms such as non-crystalline or amorphous forms, crystalline forms may provide desired or suitable hygroscopicity, particle size controls, dissolution rate, solubility, purity, physical and chemical stability, manufacturability, yield, and/or process control. Thus, solid forms of the compound of Formula I may provide advantages such as improving: the manufacturing process of the compound, the stability or storability of a drug product form of the compound, the stability or storability of a drug substance of the compound and/or the bioavailability and/or stability of the compound as an active agent.

The use of certain solvents and/or processes have been found to produce different solid forms of the compound Formula I described herein which may exhibit one or more favorable characteristics described above. The processes for the preparation of the solid forms described herein and characterization of these solid forms are described in detail below.

In particular embodiments, novel solid forms, such as crystalline forms of Formula I are disclosed. In some embodiments, zwitterionic solid forms of a compound of Formula I are disclosed. In some embodiments, solid forms of a tromethamine salt of Formula I are disclosed. In some embodiments, solid forms of a p-toluene sulfonic acid salt of Formula I are disclosed.

### Formula I Zwitterion Form I

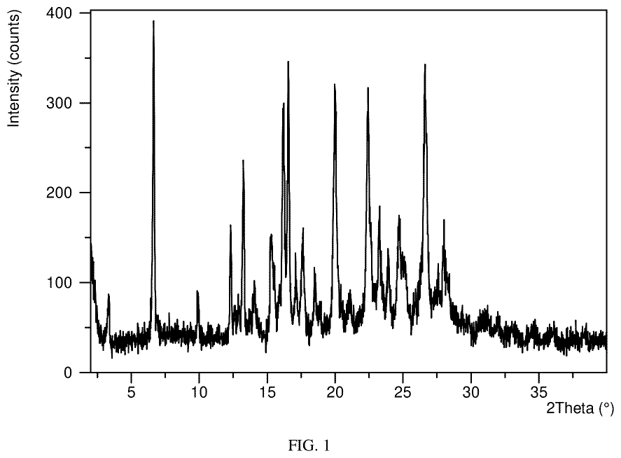

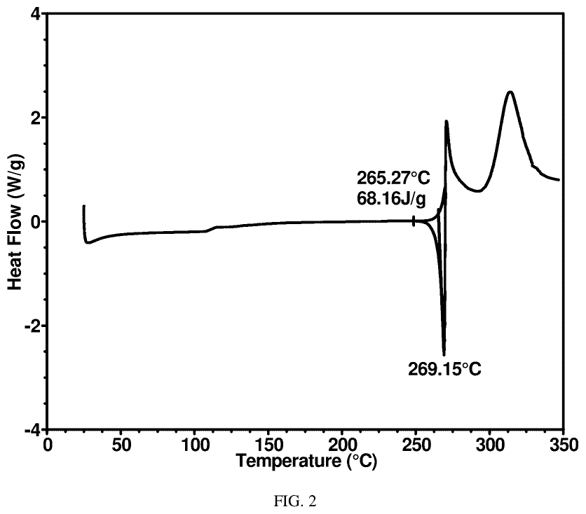

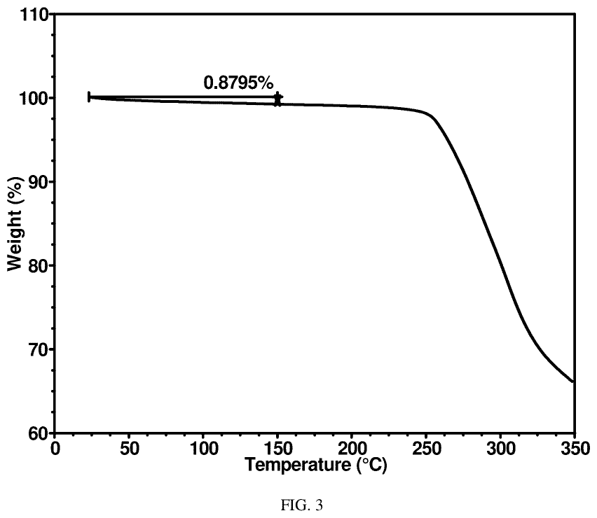

In some embodiments, provided herein is a zwitterionic solid compound of Formula I (Formula I Form I), wherein the solid form exhibits an X-ray powder diffraction (XRPD) pattern substantially as shown in FIG. 1. Zwitterionic crystalline Formula I Form I may exhibit a differential scanning calorimetry (DSC) thermogram substantially as shown in FIG. 2. Crystalline Formula I Form I may exhibit a thermogravimetric analysis (TGA) thermogram substantially as shown in FIG. 3.

In some embodiments of crystalline Formula I Form I, at least one, at least two, or all of the following (a)-(c) apply: (a) crystalline Formula I Form I has an XRPD pattern substantially as shown in FIG. 1; (b) crystalline Formula I Form I has a DSC thermogram substantially as shown in FIG. 2; (c) crystalline Formula I Form I has a TGA thermogram substantially as shown in FIG. 3.

In some embodiments, crystalline Formula I Form I has at least one, at least two, or at least three of the following properties:
(a) an XRPD pattern substantially as shown in FIG. 1
(b) a DSC thermogram substantially as shown in FIG. 2
(c) a TGA thermogram substantially as shown in FIG. 3.

In some embodiments, crystalline Formula I Form I has an XRPD pattern displaying at least two, at least three, at least four, at least five, at least six, at least seven, at least eight, or at least nine of the degree 2θ-reflections with the greatest intensity as the XRPD pattern substantially as shown in FIG. 1.

In certain embodiments, zwitterionic crystalline Formula I Form I has an XRPD pattern comprising degree 2θ-reflections (±0.2 degrees 2θ) at 6.7, 13.3, and 16.6 degrees. In some embodiments, zwitterionic Formula I Form I has an XRPD pattern comprising degree 20-reflections (±0.2 degrees 2θ) at 6.7, 13.3, and 16.6 degrees and one, two or three of the degree 2θ-reflections (±0.2 degrees 2θ) at 12.3, 16.2, and 20.0 degrees. In some embodiments, zwitterionic Formula I Form I has an XRPD pattern comprising degree 2θ-reflections (+0.2 degrees 2θ) at 6.7, 13.3, and 16.6 degrees and one, two or three of the degree 2θ-reflections (+0.2 degrees 2θ) at 15.3, 22.4, and 26.6 degrees. In some embodiments, zwitterionic Formula I Form I has an XRPD pattern comprising degree 2θ-reflections (±0.2 degrees 2θ) at 6.7, 13.3, 16.6, 12.3, 16.2, 20.0, 15.3, 22.4, and 26.6 degrees.

In some embodiments, zwitterionic Formula I Form I has a differential scanning calorimetry thermogram comprising, an endotherm with an onset at about 265° C.

### Formula I Zwitterion Form II

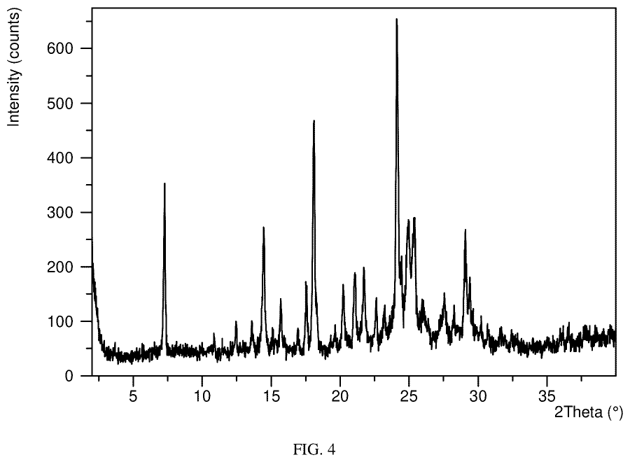

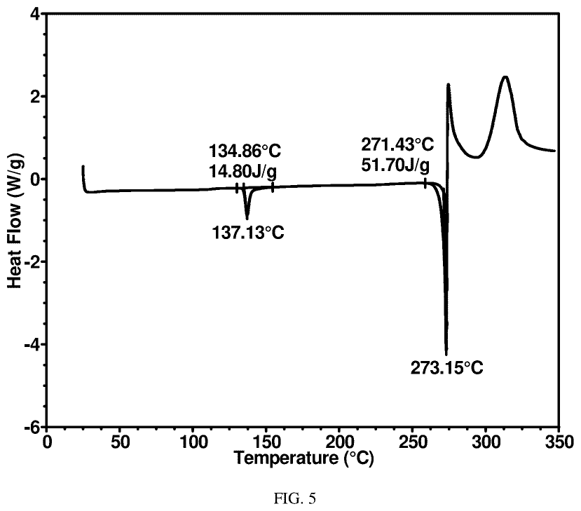

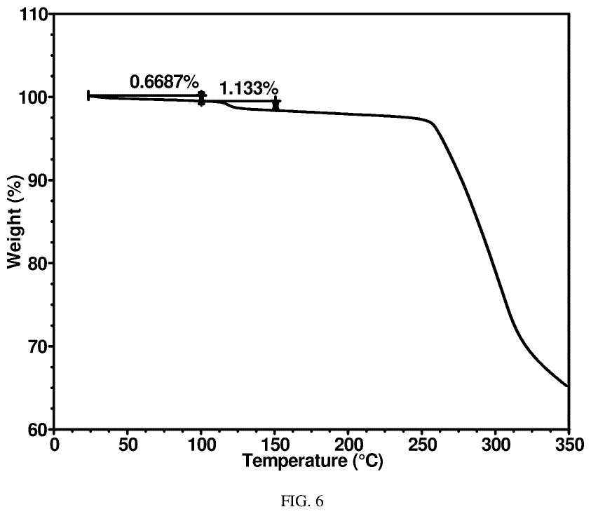

In some embodiments, provided herein is a zwitterionic crystalline compound of Formula I (Formula I Form II), wherein the crystal structure exhibits an X-ray powder diffraction (XRPD) pattern substantially as shown in FIG. 4. Zwitterionic crystalline Formula I Form II may exhibit a differential scanning calorimetry (DSC) thermogram substantially as shown in FIG. 5. Crystalline Formula I Form I may exhibit a thermogravimetric analysis (TGA) thermogram substantially as shown in FIG. 6.

In some embodiments of crystalline Formula I Form II, at least one, at least two, or all of the following (a)-(c) apply: (a) crystalline Formula I Form II has an XRPD pattern substantially as shown in FIG. 4; (b) crystalline Formula I Form II has a DSC thermogram substantially as shown in FIG. 5; (c) crystalline Formula I Form II has a TGA thermogram substantially as shown in FIG. 6.

In some embodiments, crystalline Formula I Form II has at least one, at least two, or at least three of the following properties:
(a) an XRPD pattern substantially as shown in FIG. 4
(b) a DSC thermogram substantially as shown in FIG. 5
(c) a TGA thermogram substantially as shown in FIG. 6.

In some embodiments, crystalline Formula I Form II has an XRPD pattern displaying at least two, at least three, at least four, at least five, at least six, at least seven, at least eight, or at least nine of the degree 2θ-reflections with the greatest intensity as the XRPD pattern substantially as shown in FIG. 4.

In certain embodiments, zwitterionic crystalline Formula I Form II has an XRPD pattern comprising degree 2θ-reflections (±0.2 degrees 2θ) at 7.3, 18.1, and 24.1 degrees. In some embodiments, zwitterionic Formula I Form II has an XRPD pattern comprising degree 20-reflections (±0.2 degrees 2θ) at 7.3, 18.1, and 24.1 degrees and one, two, three, or four of the degree 2θ-reflections (±0.2 degrees 2θ) at 14.5, 25.0, 25.4, and 29.1 degrees. In some embodiments, zwitterionic Formula I Form II has an XRPD pattern comprising degree 20-reflections (±0.2 degrees 2θ) at 7.3, 18.1, 24.1, 14.5, 25.0, 25.4, and 29.1 degrees.

In some embodiments, zwitterionic Formula I Form II has a differential scanning calorimetry thermogram having an endotherm with onset at about 135° C. In some embodiments, zwitterionic Formula I Form II has a differential scanning calorimetry thermogram having an endotherm with onset at about 271° C.

### Formula I Zwitterion Hydrate

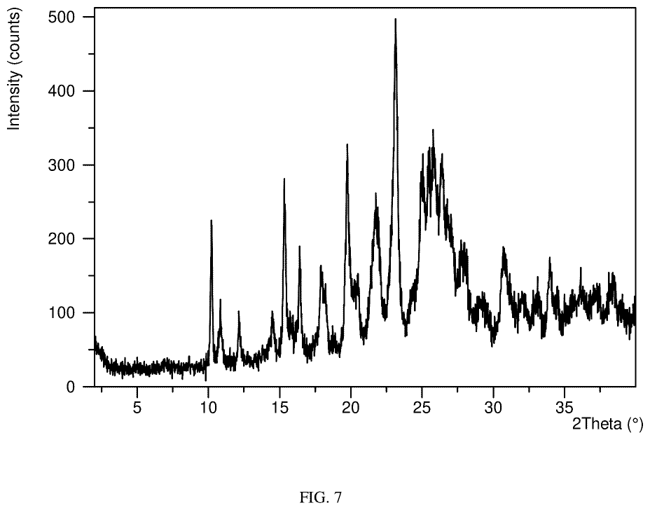

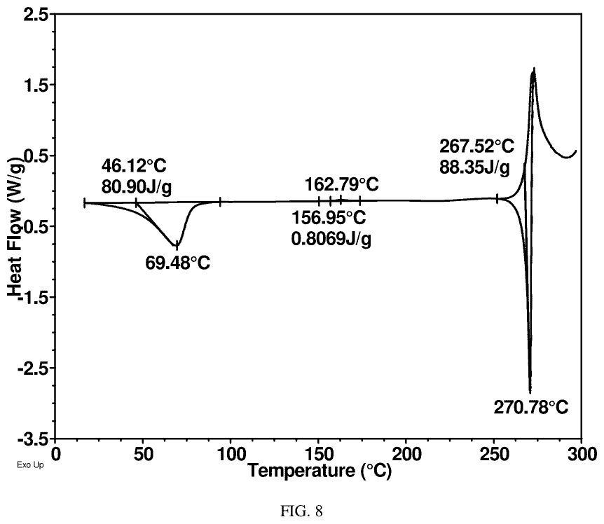

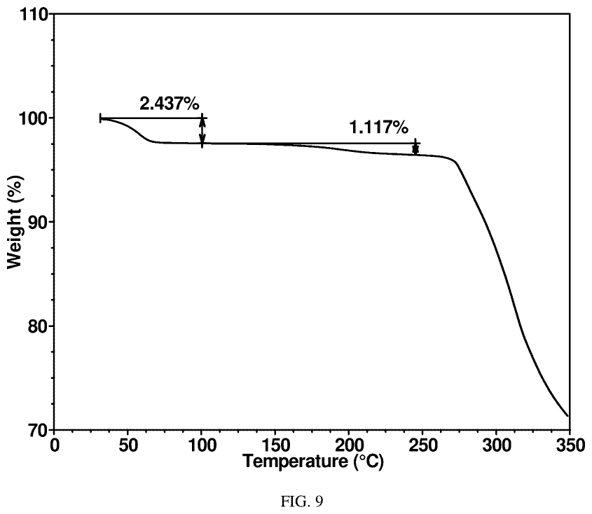

In some embodiments, provided herein is a zwitterionic crystalline compound of Formula I (Formula I hydrate), wherein the crystal structure exhibits an X-ray powder diffraction (XRPD) pattern substantially as shown in FIG. 7. Zwitterionic crystalline Formula I hydrate may exhibit a differential scanning calorimetry (DSC) thermogram substantially as shown in FIG. 8. Crystalline Formula I hydrate may exhibit a thermogravimetric analysis (TGA) thermogram substantially as shown in FIG. 9.

In some embodiments of crystalline Formula I hydrate, at least one, at least two, or all of the following (a)-(c) apply: (a) crystalline Formula I hydrate has an XRPD pattern substantially as shown in FIG. 7; (b) crystalline Formula I hydrate has a DSC thermogram substantially as shown in FIG. 8; (c) crystalline Formula I hydrate has a TGA thermogram substantially as shown in FIG. 9.

In some embodiments, crystalline Formula I hydrate has at least one, at least two, or at least three of the following properties:
(a) an XRPD pattern substantially as shown in FIG. 7
(b) a DSC thermogram substantially as shown in FIG. 8
(c) a TGA thermogram substantially as shown in FIG. 9.

In some embodiments, crystalline Formula I hydrate has an XRPD pattern displaying at least two, at least three, at least four, at least five, at least six, at least seven, at least eight, or at least nine of the degree 2θ-reflections with the greatest intensity as the XRPD pattern substantially as shown in FIG. 7.

In certain embodiments, zwitterionic crystalline Formula I hydrate has an XRPD pattern comprising degree 2θ-reflections (±0.2 degrees 2θ) at 10.2, 15.4, 19.7, and 23.1 degrees. In some embodiments, zwitterionic Formula I hydrate has an XRPD pattern comprising degree 2θ-reflections (±0.2 degrees 2θ) at 10.2, 15.4, 19.7, and 23.1 degrees and one, two, three, or four of the degree 2θ-reflections (±0.2 degrees 2θ) at 16.4, 17.9, 21.7, and 25.8 degrees. In some embodiments, zwitterionic Formula I hydrate has an XRPD pattern comprising degree 20-reflections (±0.2 degrees 2θ) at 10.2, 15.4, 19.7, and 23.1 degrees and one, two, or three of the degree 2θ-reflections (±0.2 degrees 2θ) at 10.8, 25.0, and 26.4 degrees. In some embodiments, zwitterionic Formula I hydrate has an XRPD pattern comprising degree 2θ-reflections (+0.2 degrees 2θ) at 10.2, 15.4, 19.7, 23.1, 16.4, 17.9, 21.7, 25.8, 10.8, 25.0, and 26.4 degrees.

In some embodiments, zwitterionic Formula I hydrate has a differential scanning calorimetry thermogram having a broad endotherm with onset at about 46° C. In some embodiments, zwitterionic Formula I hydrate has a differential scanning calorimetry thermogram having an exotherm with onset at about 157° C. In some embodiments, zwitterionic Formula I hydrate has a differential scanning calorimetry thermogram having an endotherm with onset at about 268° C.

### Formula I Zwitterion Amorphous

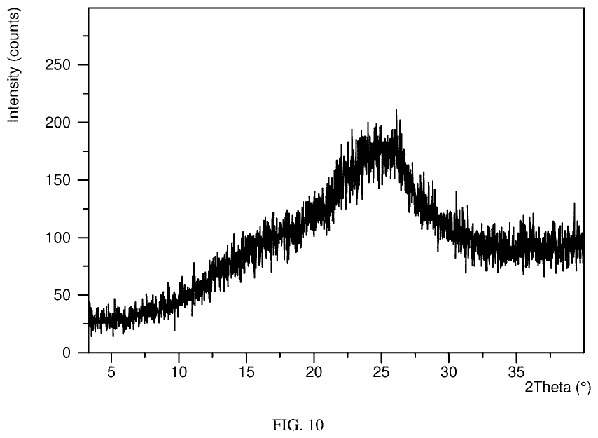

In some embodiments, provided herein is a solid zwitterionic compound of Formula I (Formula I amorphous), wherein the solid form exhibits an X-ray powder diffraction (XRPD) pattern substantially as shown in FIG. 10.

### Formula I Tromethamine Salt Form I

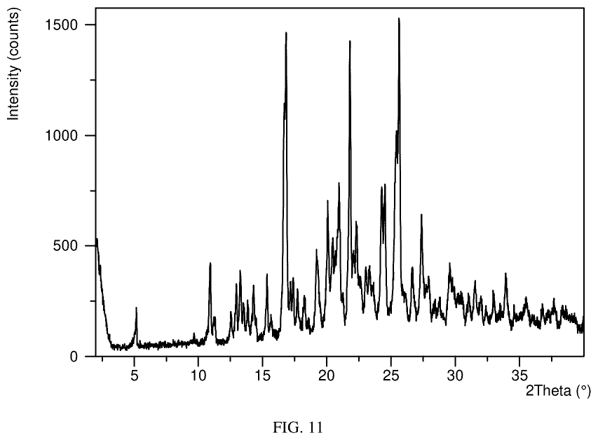

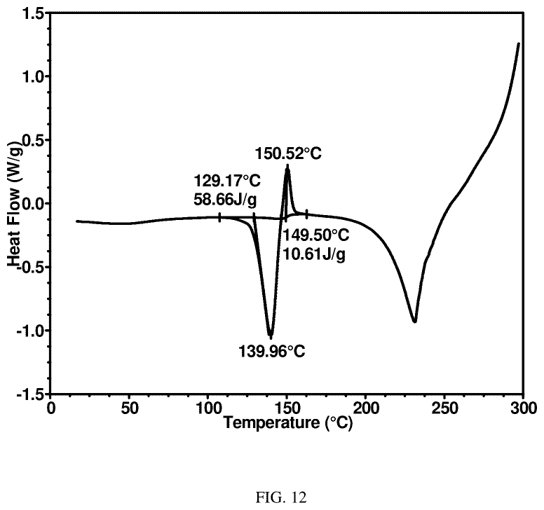

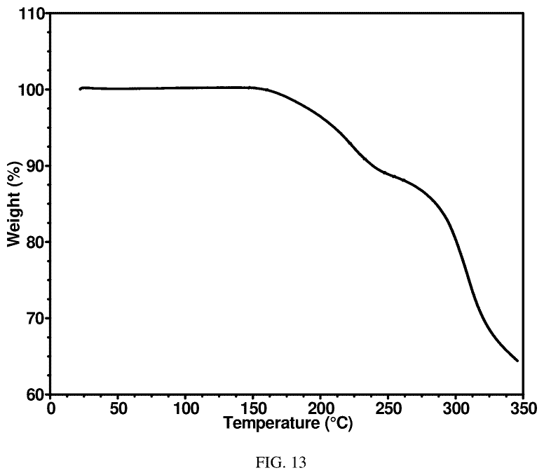

In some embodiments, provided herein is a crystalline compound of Formula I tromethamine salt Form I (Formula I tromethamine salt Form I), wherein the crystal structure exhibits an X-ray powder diffraction (XRPD) pattern substantially as shown in FIG. 11. Formula I tromethamine salt Form I may exhibit a differential scanning calorimetry (DSC) thermogram substantially as shown in FIG. 12. Crystalline Formula I tromethamine salt Form I may exhibit a thermogravimetric analysis (TGA) thermogram substantially as shown in FIG. 13.

In some embodiments of crystalline Formula I tromethamine salt Form I, at least one, at least two, or all of the following (a)-(c) apply: (a) crystalline Formula I tromethamine salt Form I has an XRPD pattern substantially as shown in FIG. 11; (b) crystalline Formula I tromethamine salt Form I has a DSC thermogram substantially as shown in FIG. 12; (c) crystalline Formula I tromethamine salt Form I has a TGA thermogram substantially as shown in FIG. 13.

In some embodiments, crystalline Formula I tromethamine salt Form I has at least one, at least two, or at least three of the following properties:
(a) an XRPD pattern substantially as shown in FIG. 11
(b) a DSC thermogram substantially as shown in FIG. 12
(c) a TGA thermogram substantially as shown in FIG. 13.

In some embodiments, crystalline Formula I tromethamine salt Form I has an XRPD pattern displaying at least two, at least three, at least four, at least five, at least six, at least seven, at least eight, or at least nine of the degree 2θ-reflections with the greatest intensity as the XRPD pattern substantially as shown in FIG. 11.

In certain embodiments, crystalline Formula I tromethamine salt Form I has an XRPD pattern comprising degree 2θ-reflections (±0.2 degrees 2θ) at 5.2, 16.8, and 25.6 degrees.

In some embodiments, Formula I tromethamine salt Form I has an XRPD pattern comprising degree 2θ-reflections (±0.2 degrees 2θ) at 5.2, 16.8, and 25.6 degrees and one, two, or three of the degree 2θ-reflections (±0.2 degrees 2θ) at 10.9, 15.3, and 21.8 degrees.

In some embodiments, Formula I tromethamine salt Form I has an XRPD pattern comprising degree 2θ-reflections (±0.2 degrees 2θ) at 5.2, 16.8, and 25.6 degrees and one, two, three, four, or five of the degree 2θ-reflections (±0.2 degrees 2θ) at 13.3, 20.1, 20.4, 21.0, and 24.3 degrees.

In some embodiments, Formula I tromethamine salt Form I has an XRPD pattern comprising degree 2θ-reflections (±0.2 degrees 2θ) at 5.2, 16.8, 25.6, 10.9, 15.3, 21.8, and 13.3, 20.1, 20.4, 21.0, and 24.3.

In some embodiments, Formula I tromethamine salt Form I has a differential scanning calorimetry thermogram having an endotherm with onset at about 129° C. In some embodiments, Formula I tromethamine salt Form I has a differential scanning calorimetry thermogram having an exotherm with onset at about 150° C.

### Formula I Tromethamine Salt Form II

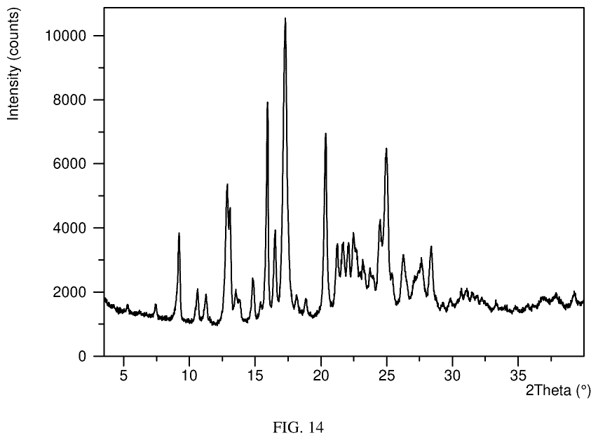

In some embodiments, provided herein is a crystalline compound of Formula I tromethamine salt Form II (Formula I tromethamine salt Form II), wherein the crystal structure exhibits an X-ray powder diffraction (XRPD) pattern substantially as shown in FIG. 14.

In some embodiments, crystalline Formula I tromethamine salt Form II has an XRPD pattern displaying at least two, at least three, at least four, at least five, at least six, at least seven, at least eight, or at least nine of the degree 2θ-reflections with the greatest intensity as the XRPD pattern substantially as shown in FIG. 14.

In certain embodiments, crystalline Formula I tromethamine salt Form II has an XRPD pattern comprising degree 2θ-reflections (±0.2 degrees 2θ) at 9.2, 15.9, and 17.3 degrees. In some embodiments, Formula I tromethamine salt Form II has an XRPD pattern comprising degree 2θ-reflections (±0.2 degrees 2θ) at 9.2, 15.9, and 17.3 degrees and one, two, three, or four of the degree 2θ-reflections (±0.2 degrees 2θ) at 12.9, 20.4, 24.5, and 25.1 degrees. In some embodiments, Formula I tromethamine salt Form II has an XRPD pattern comprising degree 2θ-reflections (±0.2 degrees 2θ) at 9.2, 15.9, 17.3, 12.9, 20.4, 24.5, and 25.1 degrees.

### Formula I Tromethamine Salt Hydrate I

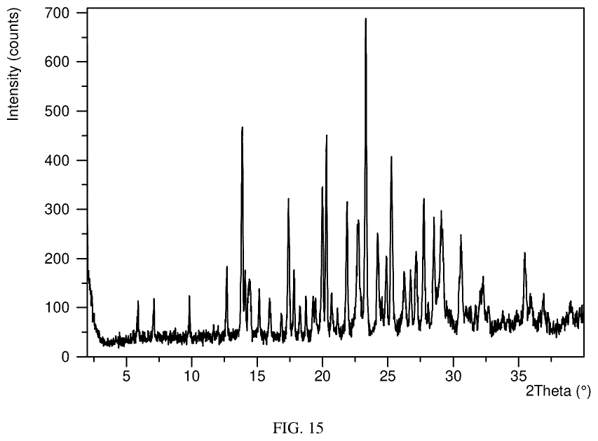

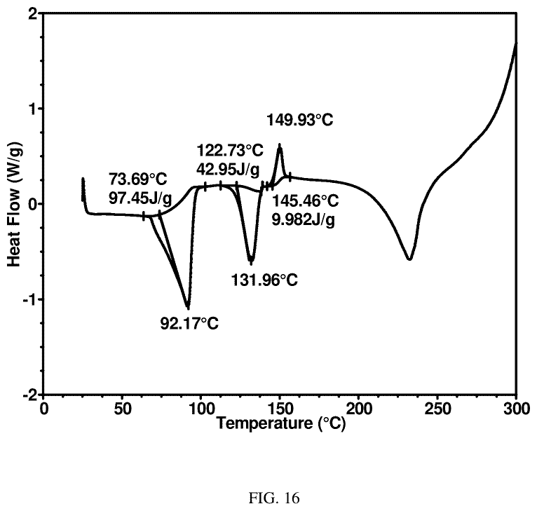

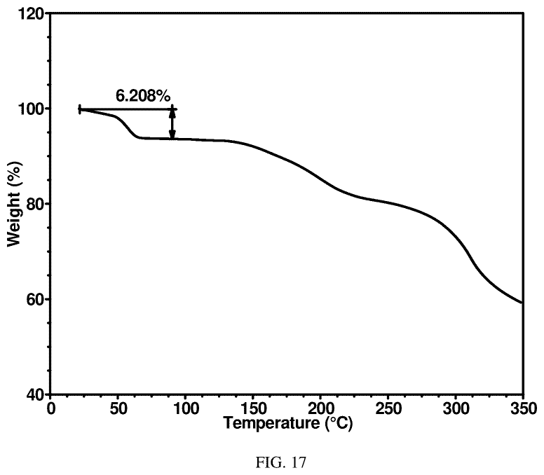

In some embodiments, provided herein is a crystalline compound of Formula I tromethamine salt hydrate I (Formula I tromethamine salt hydrate I), wherein the crystal structure exhibits an X-ray powder diffraction (XRPD) pattern substantially as shown in FIG. 15. Formula I tromethamine salt hydrate I may exhibit a differential scanning calorimetry (DSC) thermogram substantially as shown in FIG. 16. Crystalline Formula I tromethamine salt hydrate I may exhibit a thermogravimetric analysis (TGA) thermogram substantially as shown in FIG. 17.

In some embodiments of crystalline Formula I tromethamine salt hydrate I, at least one, at least two, or all of the following (a)-(c) apply: (a) crystalline Formula I tromethamine salt hydrate I has an XRPD pattern substantially as shown in FIG. 15; (b) crystalline Formula I tromethamine salt hydrate I has a DSC thermogram substantially as shown in FIG. 16; (c) crystalline Formula I tromethamine salt hydrate I has a TGA thermogram substantially as shown in FIG. 17.

In some embodiments, crystalline Formula I tromethamine salt hydrate I has at least one, at least two, or at least three of the following properties:
(a) an XRPD pattern substantially as shown in FIG. 15
(b) a DSC thermogram substantially as shown in FIG. 16
(c) a TGA thermogram substantially as shown in FIG. 17.

In some embodiments, crystalline Formula I tromethamine salt hydrate I has an XRPD pattern displaying at least two, at least three, at least four, at least five, at least six, at least seven, at least eight, or at least nine of the degree 2θ-reflections with the greatest intensity as the XRPD pattern substantially as shown in FIG. 15.

In certain embodiments, crystalline Formula I tromethamine salt hydrate I has an XRPD pattern comprising degree 2θ-reflections (±0.2 degrees 2θ) at 5.9, 13.9, and 23.3 degrees. In some embodiments, Formula I tromethamine salt hydrate I has an XRPD pattern comprising degree 2θ-reflections (±0.2 degrees 2θ) at 5.9, 13.9, and 23.3 degrees and one, two, or three of the degree 2θ-reflections (±0.2 degrees 2θ) at 7.1, 12.7, and 20.3 degrees. In some embodiments, Formula I tromethamine salt hydrate I has an XRPD pattern comprising degree 2θ-reflections (±0.2 degrees 2θ) at 5.9, 13.9, and 23.3 degrees and one, two, three, or four of the degree 2θ-reflections (±0.2 degrees 2θ) at 9.8, 17.4, 22.7, and 25.3 degrees. In some embodiments, Formula I tromethamine salt hydrate I has an XRPD pattern comprising degree 2θ-reflections (±0.2 degrees 2θ) at 5.9, 13.9, 23.3, 7.1, 12.7, 20.3, 9.8, 17.4, 22.7, and 25.3 degrees.

In some embodiments, Formula I tromethamine salt hydrate I has a differential scanning calorimetry thermogram having an endotherm with onset at about 74° C. In some embodiments, Formula I tromethamine salt hydrate I having endotherm with onset at about 123° C. In some embodiments, Formula I tromethamine salt hydrate I has a differential scanning calorimetry thermogram having an exotherm with onset at about 145° C.

### Formula I Tromethamine Salt Hydrate II

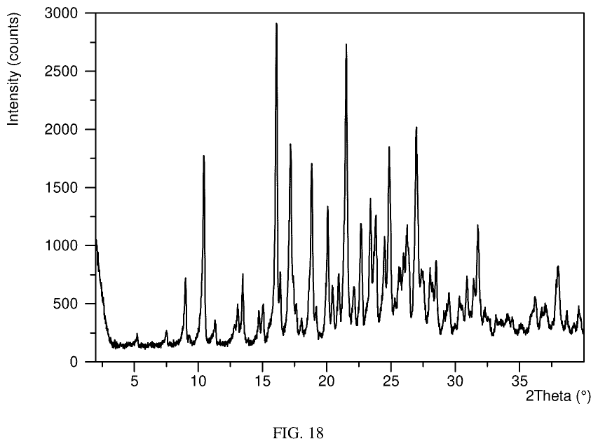

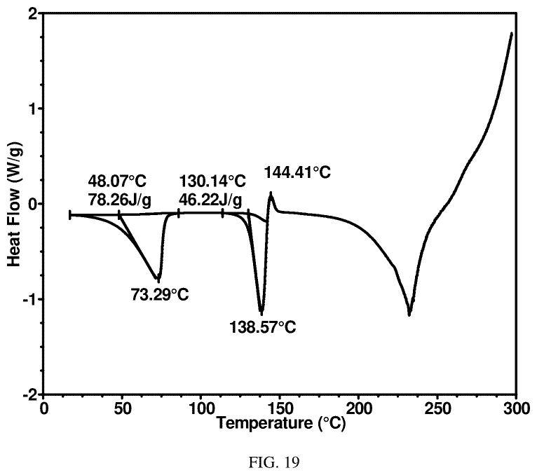

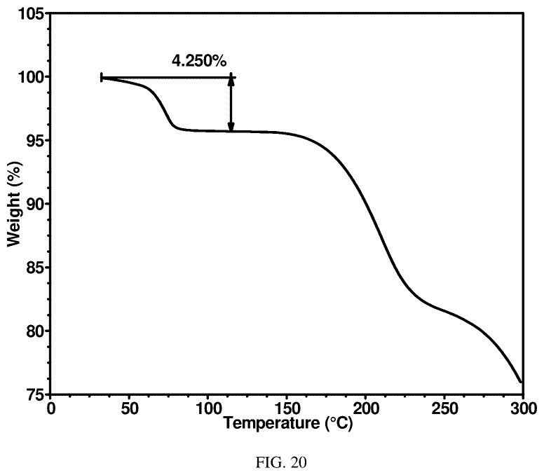

In some embodiments, provided herein is a crystalline compound of Formula I tromethamine salt hydrate II (Formula I tromethamine salt hydrate II), wherein the crystal structure exhibits an X-ray powder diffraction (XRPD) pattern substantially as shown in FIG. 18. Formula I tromethamine salt hydrate II may exhibit a differential scanning calorimetry (DSC) thermogram substantially as shown in FIG. 19. Crystalline Formula I tromethamine salt hydrate II may exhibit a thermogravimetric analysis (TGA) thermogram substantially as shown in FIG. 20.

In some embodiments of crystalline Formula I tromethamine salt hydrate II, at least one, at least two, or all of the following (a)-(c) apply: (a) crystalline Formula I tromethamine salt hydrate II has an XRPD pattern substantially as shown in FIG. 18; (b) crystalline Formula I tromethamine salt hydrate II has a DSC thermogram substantially as shown in FIG. 19; (c) crystalline Formula I tromethamine salt hydrate II has a TGA thermogram substantially as shown in FIG. 20.

In some embodiments, crystalline Formula I tromethamine salt hydrate II has at least one, at least two, or at least three of the following properties:
(a) an XRPD pattern substantially as shown in FIG. 18
(b) a DSC thermogram substantially as shown in FIG. 19
(c) a TGA thermogram substantially as shown in FIG. 20.

In some embodiments, crystalline Formula I tromethamine salt hydrate II has an XRPD pattern displaying at least two, at least three, at least four, at least five, at least six, at least seven, at least eight, or at least nine of the degree 2θ-reflections with the greatest intensity as the XRPD pattern substantially as shown in FIG. 18.

In certain embodiments, crystalline Formula I tromethamine salt hydrate II has an XRPD pattern comprising degree 2θ-reflections (±0.2 degrees 2θ) at 10.5, 16.1, and 21.5 degrees. In some embodiments, Formula I tromethamine salt hydrate II has an XRPD pattern comprising degree 2θ-reflections (±0.2 degrees 2θ) at 10.5, 16.1, and 21.5 degrees and one, two, or three of the degree 2θ-reflections (±0.2 degrees 2θ) at 9.0, 17.2, and 18.8 degrees. In some embodiments, Formula I tromethamine salt hydrate II has an XRPD pattern comprising degree 2θ-reflections (±0.2 degrees 2θ) at 10.5, 16.1, and 21.5 degrees and one, two, three, or four of the degree 2θ-reflections (±0.2 degrees 2θ) at 13.4, 20.1, 24.9, and 26.9 degrees. In some embodiments, Formula I tromethamine salt hydrate II has an XRPD pattern comprising degree 2θ-reflections (±0.2 degrees 2θ) at 10.5, 16.1, 21.5, 9.0, 17.2, 18.8, 13.4, 20.1, 24.9, and 26.9 degrees.

In some embodiments, Formula I tromethamine salt hydrate II has a differential scanning calorimetry thermogram having a broad endotherm with onset at about 48° C. In some embodiments, Formula I tromethamine salt hydrate II has a differential scanning calorimetry thermogram having an endotherm with onset at about 130° C.

### Formula I Tromethamine Salt Hydrate III

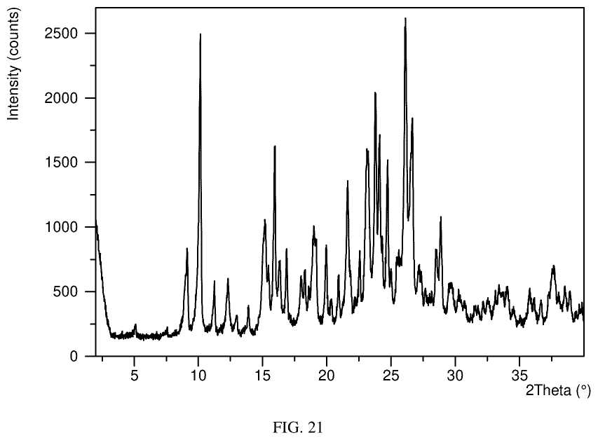

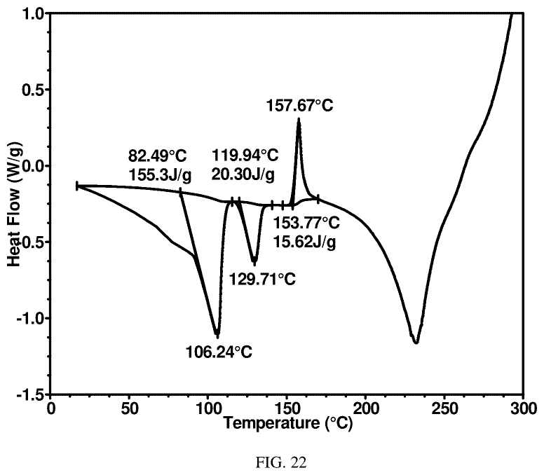

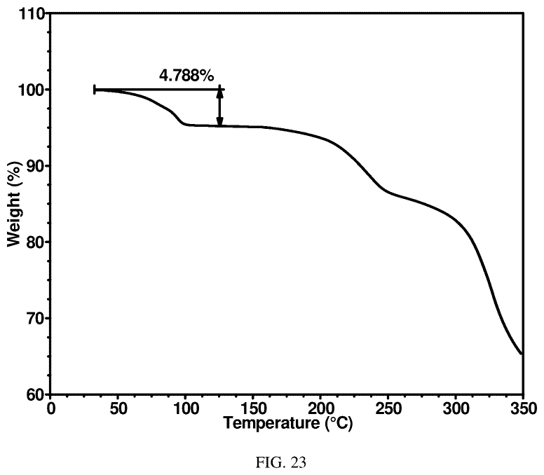

In some embodiments, provided herein is a crystalline compound of Formula I tromethamine salt hydrate III (Formula I tromethamine salt hydrate III), wherein the crystal structure exhibits an X-ray powder diffraction (XRPD) pattern substantially as shown in FIG. 21. Formula I tromethamine salt hydrate III may exhibit a differential scanning calorimetry (DSC) thermogram substantially as shown in FIG. 22. Crystalline Formula I tromethamine salt hydrate III may exhibit a thermogravimetric analysis (TGA) thermogram substantially as shown in FIG. 23.

In some embodiments of crystalline Formula I tromethamine salt hydrate III, at least one, at least two, or all of the following (a)-(c) apply: (a) crystalline Formula I tromethamine salt hydrate III has an XRPD pattern substantially as shown in FIG. 21; (b) crystalline Formula I tromethamine salt hydrate III has a DSC thermogram substantially as shown in FIG. 22; (c) crystalline Formula I tromethamine salt hydrate III has a TGA thermogram substantially as shown in FIG. 23.

In some embodiments, crystalline Formula I tromethamine salt hydrate III has at least one, at least two, or at least three of the following properties:
(a) an XRPD pattern substantially as shown in FIG. 21
(b) a DSC thermogram substantially as shown in FIG. 22
(c) a TGA thermogram substantially as shown in FIG. 23.

In some embodiments, crystalline Formula I tromethamine salt hydrate III has an XRPD pattern displaying at least two, at least three, at least four, at least five, at least six, at least seven, at least eight, or at least nine of the degree 2θ-reflections with the greatest intensity as the XRPD pattern substantially as shown in FIG. 21.

In certain embodiments, crystalline Formula I tromethamine salt hydrate III has an XRPD pattern comprising degree 2θ-reflections (±0.2 degrees 2θ) at 10.2, 16.0, and 23.2 degrees. In some embodiments, Formula I tromethamine salt hydrate III has an XRPD pattern comprising degree 2θ-reflections (±0.2 degrees 2θ) at 10.2, 16.0, and 23.2 degrees and one, two, or three of the degree 2θ-reflections (±0.2 degrees 2θ) at 9.1, 15.2, and 23.8 degrees. In some embodiments, Formula I tromethamine salt hydrate III has an XRPD pattern comprising degree 2θ-reflections (±0.2 degrees 2θ) at 10.2, 16.0, and 23.2 degrees and one, two, or three of the degree 2θ-reflections (±0.2 degrees 2θ) at 11.2, 12.3, 19.0, 21.6, and 26.1 degrees. In some embodiments, Formula I tromethamine salt hydrate III has an XRPD pattern comprising degree 2θ-reflections (±0.2 degrees 2θ) at 10.2, 16.0, 23.2, 9.1, 15.2, 23.8, 11.2, 12.3, 19.0, 21.6, and 26.1 degrees.

In some embodiments, Formula I tromethamine salt hydrate III has a differential scanning calorimetry thermogram having a broad endotherm with onset at about 82° C. In some embodiments, Formula I tromethamine salt hydrate III has a differential scanning calorimetry thermogram having an endotherm with onset at about 120° C. In some embodiments, Formula I tromethamine salt hydrate III has a differential scanning calorimetry thermogram having an exotherm with onset at about 154° C.

### Formula I Tromethamine Salt Hydrate IV

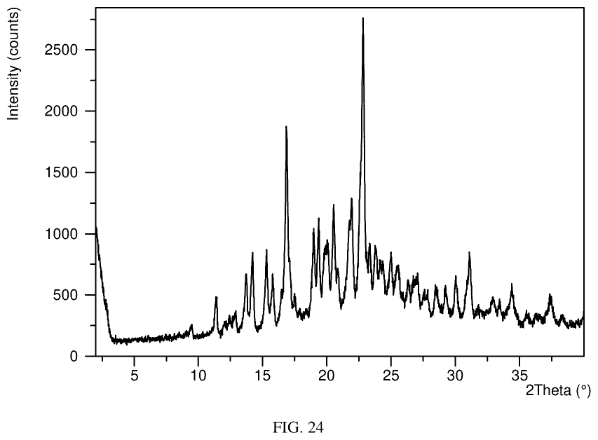

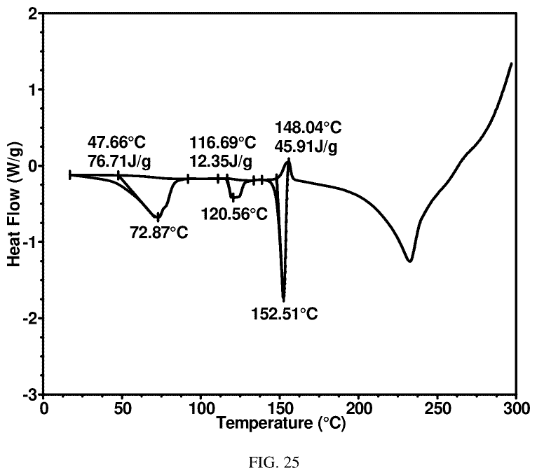

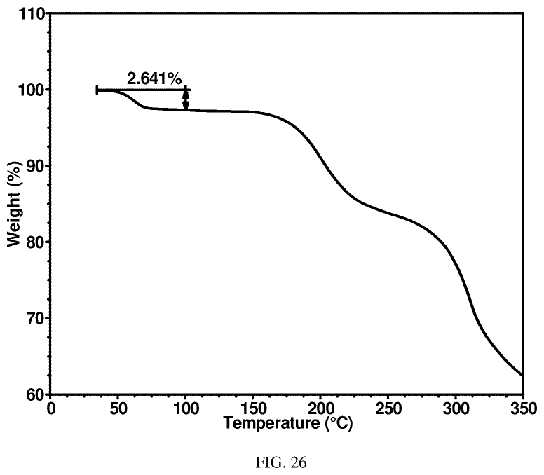

In some embodiments, provided herein is a crystalline compound of Formula I tromethamine salt hydrate IV (Formula I tromethamine salt hydrate IV), wherein the crystal structure exhibits an X-ray powder diffraction (XRPD) pattern substantially as shown in FIG. 24. Formula I tromethamine salt hydrate IV may exhibit a differential scanning calorimetry (DSC) thermogram substantially as shown in FIG. 25. Crystalline Formula I tromethamine salt hydrate IV may exhibit a thermogravimetric analysis (TGA) thermogram substantially as shown in FIG. 26.

In some embodiments of crystalline Formula I tromethamine salt hydrate IV, at least one, at least two, or all of the following (a)-(c) apply: (a) crystalline Formula I tromethamine salt hydrate IV has an XRPD pattern substantially as shown in FIG. 24; (b) crystalline Formula I tromethamine salt hydrate IV has a DSC thermogram substantially as shown in FIG. 25; (c) crystalline Formula I tromethamine salt hydrate IV has a TGA thermogram substantially as shown in FIG. 26.

In some embodiments, crystalline Formula I tromethamine salt hydrate IV has at least one, at least two, or at least three of the following properties:
(a) an XRPD pattern substantially as shown in FIG. 24
(b) a DSC thermogram substantially as shown in FIG. 25
(c) a TGA thermogram substantially as shown in FIG. 26.

In some embodiments, crystalline Formula I tromethamine salt hydrate IV has an XRPD pattern displaying at least two, at least three, at least four, at least five, at least six, at least seven, at least eight, or at least nine of the degree 2θ-reflections with the greatest intensity as the XRPD pattern substantially as shown in FIG. 24.

In certain embodiments, crystalline Formula I tromethamine salt hydrate IV has an XRPD pattern comprising degree 2θ-reflections (±0.2 degrees 2θ) at 11.4, 16.9, and 22.8 degrees.

In some embodiments, Formula I tromethamine salt hydrate IV has an XRPD pattern comprising degree 2θ-reflections (±0.2 degrees 2θ) at 11.4, 16.9, and 22.8 degrees and one, two, or three of the degree 2θ-reflections (±0.2 degrees 2θ) at 14.2, 15.3, and 20.5 degrees.

In some embodiments, Formula I tromethamine salt hydrate IV has an XRPD pattern comprising degree 2θ-reflections (±0.2 degrees 2θ) at 11.4, 16.9, and 22.8 degrees and one, two, three, or four of the degree 2θ-reflections (±0.2 degrees 2θ) at 13.7, 15.8, 19.4, and 21.9 degrees.

In some embodiments, Formula I tromethamine salt hydrate IV has an XRPD pattern comprising degree 2θ-reflections (±0.2 degrees 2θ) at 11.4, 16.9, 22.8, 14.2, 15.3, 20.5, 13.7, 15.8, 19.4, and 21.9 degrees.

In some embodiments, Formula I tromethamine salt hydrate IV has a differential scanning calorimetry thermogram having a broad endotherm with onset at about 48° C. In some embodiments, Formula I tromethamine salt hydrate IV has a differential scanning calorimetry thermogram having an endotherm with onset at about 117° C. In some embodiments, Formula I tromethamine salt hydrate IV has a differential scanning calorimetry thermogram having an endotherm with onset at about 148° C.

### Formula I Tromethamine Salt Methanol Solvate I

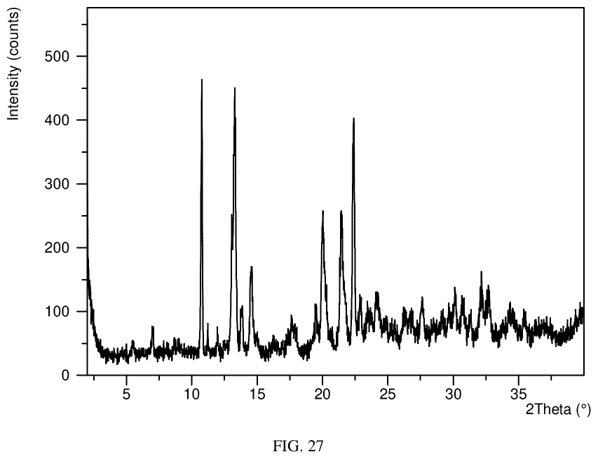

In some embodiments, provided herein is a crystalline compound of Formula I tromethamine salt methanol solvate I (Formula I tromethamine salt methanol solvate I), wherein the crystal structure exhibits an X-ray powder diffraction (XRPD) pattern substantially as shown in FIG. 27.

In some embodiments, crystalline Formula I tromethamine salt methanol solvate I has an XRPD pattern displaying at least two, at least three, at least four, at least five, at least six, at least seven, at least eight, or at least nine of the degree 2θ-reflections with the greatest intensity as the XRPD pattern substantially as shown in FIG. 27.

In certain embodiments, crystalline Formula I tromethamine salt methanol solvate I has an XRPD pattern comprising degree 2θ-reflections (±0.2 degrees 2θ) at 10.8, 13.3, and 22.3 degrees.

In some embodiments, Formula I tromethamine salt methanol solvate I has an XRPD pattern comprising degree 2θ-reflections (±0.2 degrees 2θ) at 10.8, 13.3, and 22.3 degrees and one, two, or three of the degree 2θ-reflections (±0.2 degrees 2θ) at 14.5, 20.0, and 21.4 degrees.

In some embodiments, Formula I tromethamine salt methanol solvate I has an XRPD pattern comprising degree 2θ-reflections (±0.2 degrees 2θ) at 10.8, 13.3, 14.5, 20.0, 21.4, and 22.3 degrees.

### Formula I Tromethamine Salt Methanol Solvate II

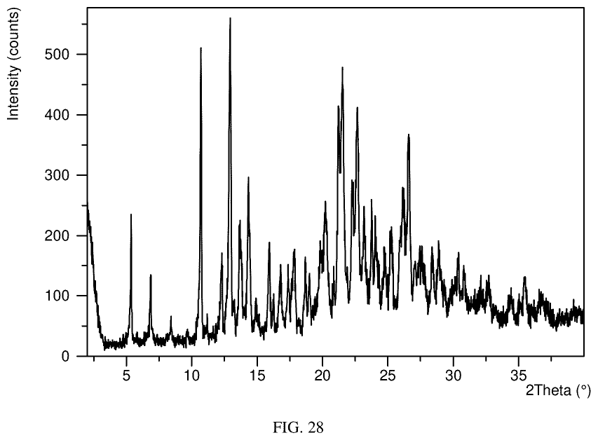

In some embodiments, provided herein is a crystalline compound of Formula I tromethamine salt methanol solvate II (Formula I tromethamine salt methanol solvate II), wherein the crystal structure exhibits an X-ray powder diffraction (XRPD) pattern substantially as shown in FIG. 28.

In some embodiments, crystalline Formula I tromethamine salt methanol solvate II has an XRPD pattern displaying at least two, at least three, at least four, at least five, at least six, at least seven, at least eight, or at least nine of the degree 2θ-reflections with the greatest intensity as the XRPD pattern substantially as shown in FIG. 28.

In certain embodiments, crystalline Formula I tromethamine salt methanol solvate II has an XRPD pattern comprising degree 2θ-reflections (±0.2 degrees 2θ) at 5.4, 12.9, and 22.7 degrees. In some embodiments, Formula I tromethamine salt methanol solvate II has an XRPD pattern comprising degree 2θ-reflections (±0.2 degrees 2θ) at 5.4, 12.9, and 22.7 degrees and one, two, or three of the degree 2θ-reflections (±0.2 degrees 2θ) at 10.7, 14.3, and 15.9 degrees. In some embodiments, Formula I tromethamine salt methanol solvate II has an XRPD pattern comprising degree 2θ-reflections (±0.2 degrees 2θ) at 5.4, 12.9, and 22.7 degrees and one or two of the degree 2θ-reflections (±0.2 degrees 2θ) at 21.2 and 26.6 degrees. In some embodiments, Formula I tromethamine salt methanol solvate II has an XRPD pattern comprising degree 2θ-reflections (±0.2 degrees 2θ) at 5.4, 10.7, 12.9, 14.3, 15.9, 21.2, 22.7, and 26.6 degrees.

### Formula I Tromethamine Salt Methanol Solvate III

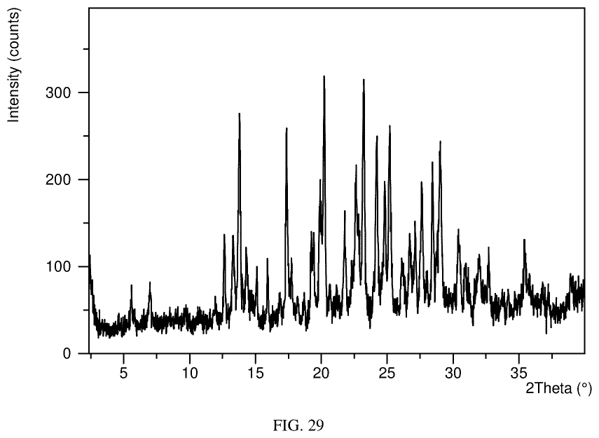

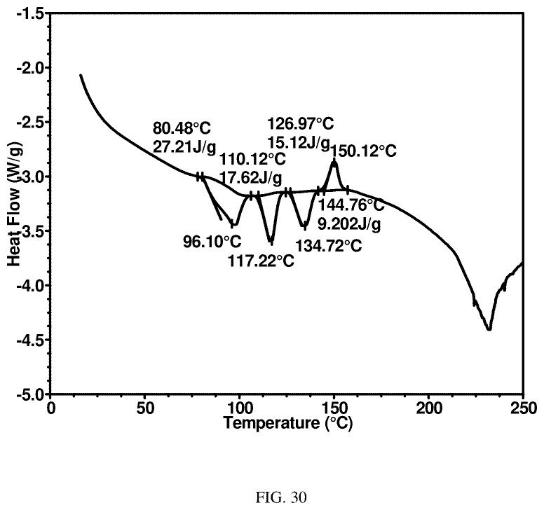

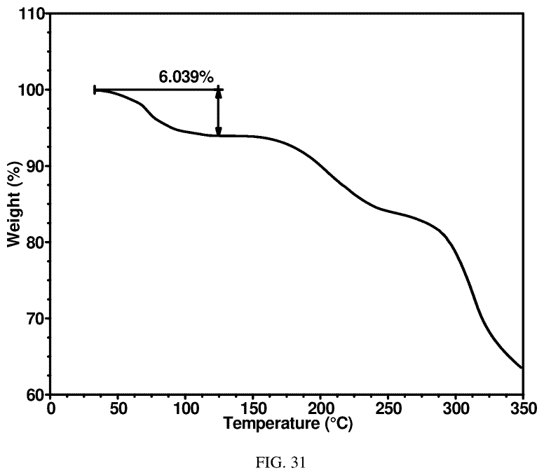

In some embodiments, provided herein is a crystalline compound of Formula I tromethamine salt methanol solvate III (Formula I tromethamine salt methanol solvate III), wherein the crystal structure exhibits an X-ray powder diffraction (XRPD) pattern substantially as shown in FIG. 29. Formula I tromethamine salt methanol solvate III may exhibit a differential scanning calorimetry (DSC) thermogram substantially as shown in FIG. 30. Crystalline Formula I tromethamine salt methanol solvate III may exhibit a thermogravimetric analysis (TGA) thermogram substantially as shown in FIG. 31.

In some embodiments of crystalline Formula I tromethamine salt methanol solvate III at least one, at least two, or all of the following (a)-(c) apply: (a) crystalline Formula I tromethamine salt methanol solvate III has an XRPD pattern substantially as shown in FIG. 29; (b) crystalline Formula I tromethamine salt methanol solvate III has a DSC thermogram substantially as shown in FIG. 30; (c) crystalline Formula I tromethamine salt methanol solvate III has a TGA thermogram substantially as shown in FIG. 31.

In some embodiments, crystalline Formula I tromethamine salt methanol solvate III has at least one, at least two, or at least three of the following properties:
(a) an XRPD pattern substantially as shown in FIG. 29
(b) a DSC thermogram substantially as shown in FIG. 30
(c) a TGA thermogram substantially as shown in FIG. 31.

In some embodiments, crystalline Formula I tromethamine salt methanol solvate III has an XRPD pattern displaying at least two, at least three, at least four, at least five, at least six, at least seven, at least eight, or at least nine of the degree 2θ-reflections with the greatest intensity as the XRPD pattern substantially as shown in FIG. 29.

In certain embodiments, crystalline Formula I tromethamine salt methanol solvate III has an XRPD pattern comprising degree 2θ-reflections (±0.2 degrees 2θ) at 12.6, 13.3, and 13.8 degrees.

In some embodiments, Formula I tromethamine salt methanol solvate III has an XRPD pattern comprising degree 2θ-reflections (±0.2 degrees 2θ) at 12.6, 13.3, and 13.8 degrees and one, two, or three of the degree 2θ-reflections (±0.2 degrees 2θ) at 14.3, 17.4, and 23.2 degrees.

In some embodiments, Formula I tromethamine salt methanol solvate III has an XRPD pattern comprising degree 2θ-reflections (±0.2 degrees 2θ) at 12.6, 13.3, and 13.8 degrees and one, two, or three of the degree 2θ-reflections (±0.2 degrees 2θ) at 20.2, 24.2, 25.2 and 29.1 degrees.

In some embodiments, Formula I tromethamine salt methanol solvate III has an XRPD pattern comprising degree 2θ-reflections (±0.2 degrees 2θ) at 12.6, 13.3, 13.8, 14.3, 17.4, 20.2, 23.2, 24.2, 25.2, and 29.1 degrees.

In some embodiments, Formula I tromethamine salt methanol solvate III has a differential scanning calorimetry thermogram having an endotherm with onset at about 80° C. In some embodiments, Formula I tromethamine salt methanol solvate III has a differential scanning calorimetry thermogram having an endotherm with onset at about 110° C. In some embodiments, Formula I tromethamine salt methanol solvate III has a differential scanning calorimetry thermogram having endotherm with onset at about 127° C. In some embodiments, Formula I tromethamine salt hydrate IV has a differential scanning calorimetry thermogram having an exotherm with onset at about 145° C.

### Formula I Tromethamine Salt MTBE Solvate

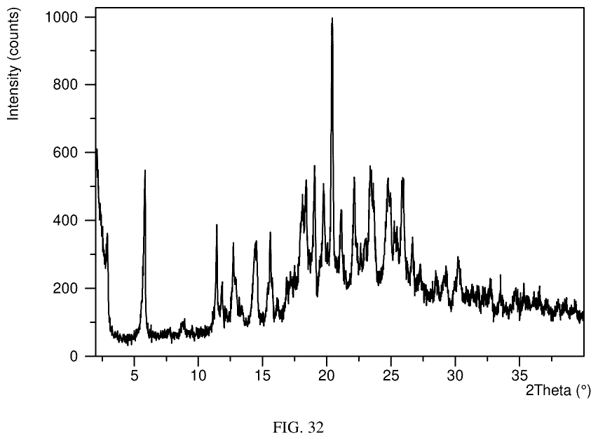

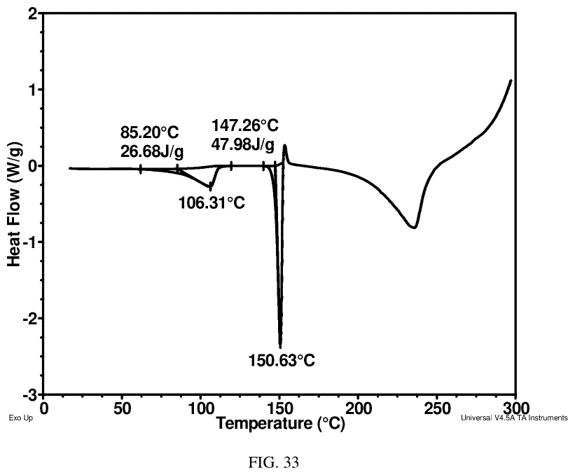

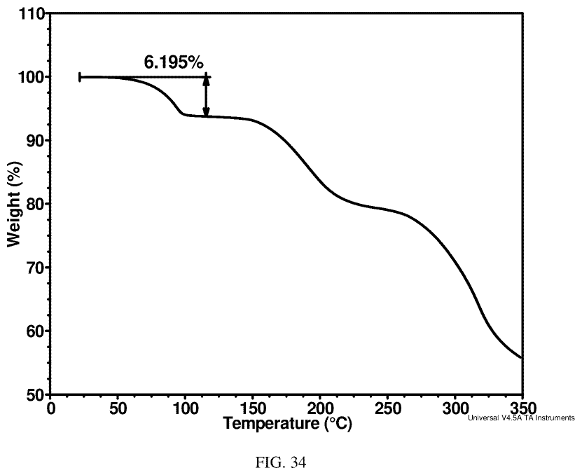

In some embodiments, provided herein is a crystalline compound of Formula I tromethamine salt methyl tertiary-butyl ether (MTBE) solvate (Formula I tromethamine salt MTBE solvate), wherein the crystal structure exhibits an X-ray powder diffraction (XRPD) pattern substantially as shown in FIG. 32. Formula I tromethamine salt MTBE solvate may exhibit a differential scanning calorimetry (DSC) thermogram substantially as shown in FIG. 33. Crystalline Formula I tromethamine salt MTBE solvate may exhibit a thermogravimetric analysis (TGA) thermogram substantially as shown in FIG. 34.

In some embodiments of crystalline Formula I tromethamine salt MTBE solvate, at least one, at least two, or all of the following (a)-(c) apply: (a) crystalline Formula I tromethamine salt MTBE solvate has an XRPD pattern substantially as shown in FIG. 32; (b) crystalline Formula I tromethamine salt MTBE solvate has a DSC thermogram substantially as shown in FIG. 33; (c) crystalline Formula I tromethamine salt MTBE solvate has a TGA thermogram substantially as shown in FIG. 34.

In some embodiments, crystalline Formula I tromethamine salt MTBE solvate has at least one, at least two, or at least three of the following properties:
(a) an XRPD pattern substantially as shown in FIG. 32
(b) a DSC thermogram substantially as shown in FIG. 33
(c) a TGA thermogram substantially as shown in FIG. 34.

In some embodiments, crystalline Formula I tromethamine salt MTBE solvate has an XRPD pattern displaying at least two, at least three, at least four, at least five, at least six, at least seven, at least eight, or at least nine of the degree 2θ-reflections with the greatest intensity as the XRPD pattern substantially as shown in FIG. 32.

In certain embodiments, crystalline Formula I tromethamine salt MTBE solvate has an XRPD pattern comprising degree 2θ-reflections (±0.2 degrees 2θ) at 5.8, 11.4, 15.6, and 20.4 degrees.

In some embodiments, Formula I tromethamine salt MTBE solvate has an XRPD pattern comprising degree 2θ-reflections (±0.2 degrees 2θ) at 5.8, 11.4, 15.6, and 20.4 degrees and one, two, three, or four of the degree 2θ-reflections (±0.2 degrees 2θ) at 14.5, 18.4, 23.4, and 25.9 degrees.

In some embodiments, Formula I tromethamine salt MTBE solvate has an XRPD pattern comprising degree 2θ-reflections (±0.2 degrees 2θ) at 5.8, 11.4, 15.6, and 20.4 degrees and one, two, or three of the degree 2θ-reflections (±0.2 degrees 2θ) at 12.7, 19.0, and 24.7 degrees.

In some embodiments, Formula I tromethamine salt MTBE solvate has an XRPD pattern comprising degree 2θ-reflections (±0.2 degrees 2θ) at 5.8, 11.4, 12.7, 14.5, 15.6, 18.4, 19.0, 20.4, 23.4, 24.7, and 25.9 degrees.

In some embodiments, Formula I tromethamine salt MTBE solvate has a differential scanning calorimetry thermogram having an endotherm with onset at about 85° C. In some embodiments, Formula I tromethamine salt MTBE solvate has a differential scanning calorimetry thermogram having endotherm with onset at about 147° C.

### Formula I Tromethamine Amorphous

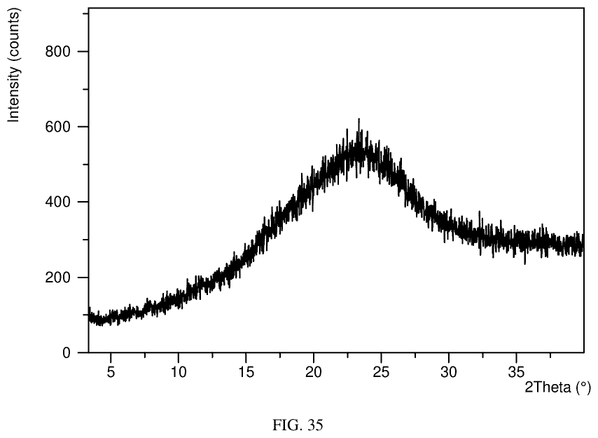

In some embodiments, provided herein is a compound of Formula I tromethamine salt amorphous form (Formula I tromethamine amorphous form), wherein the solid form exhibits an X-ray powder diffraction (XRPD) pattern substantially as shown in FIG. 35.

### Formula I Tromethamine Salt Ethanol Solvate

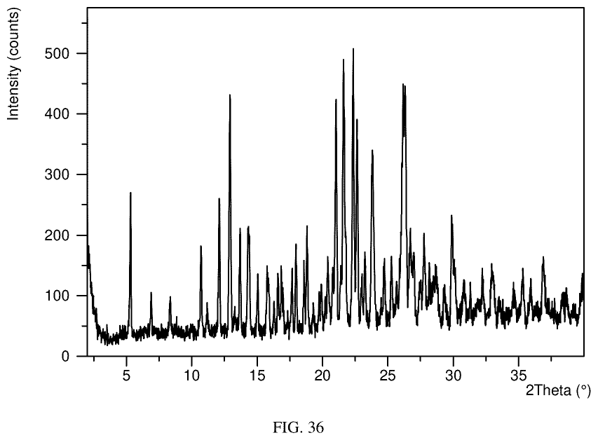

In some embodiments, provided herein is a crystalline compound of Formula I tromethamine salt ethanol solvate (Formula I tromethamine salt ethanol solvate), wherein the crystal structure exhibits an X-ray powder diffraction (XRPD) pattern substantially as shown in FIG. 36.

In some embodiments, crystalline Formula I tromethamine salt ethanol solvate form has an XRPD pattern displaying at least two, at least three, at least four, at least five, at least six, at least seven, at least eight, or at least nine of the degree 2θ-reflections with the greatest intensity as the XRPD pattern substantially as shown in FIG. 36. In some embodiments, tromethamine salt ethanol solvate has four or more of the following characteristic peaks: 5.3, 10.7, 12.1, 12.9, 21.0, 21.6, 22.3, 22.6, 23.8, and 26.3° 20.

Formula I p-Toluenesulfonic Acid (p-TSA) Salt Form I

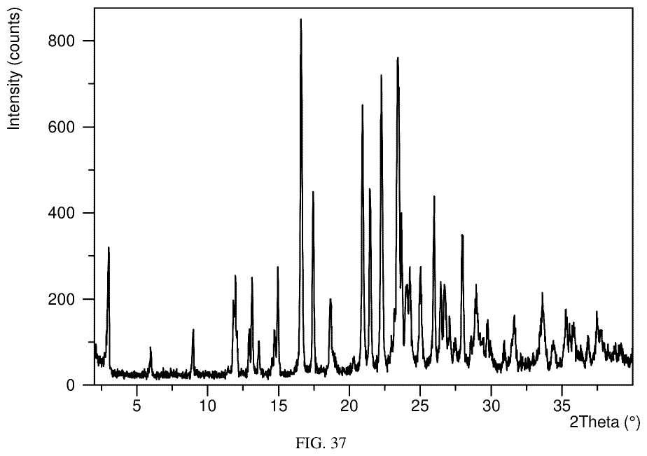

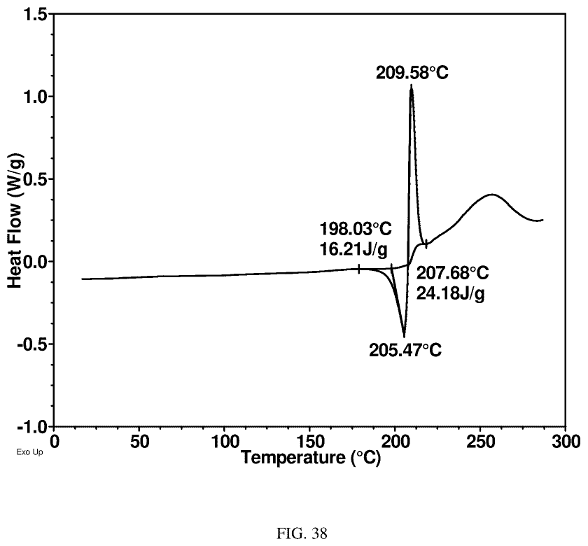

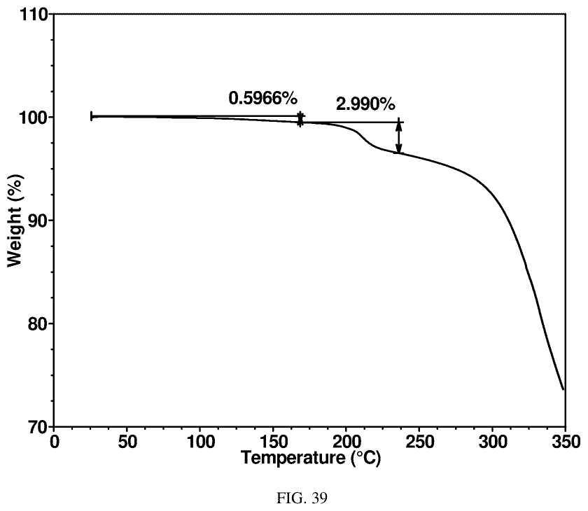

In some embodiments, provided herein is a crystalline compound of Formula I p-TSA salt Form I (Formula I p-TSA salt Form I), wherein the crystal structure exhibits an X-ray powder diffraction (XRPD) pattern substantially as shown in FIG. 37. Formula I p-TSA salt Form I may exhibit a differential scanning calorimetry (DSC) thermogram substantially as shown in FIG. 38. Crystalline Formula I p-TSA salt Form I may exhibit a thermogravimetric analysis (TGA) thermogram substantially as shown in FIG. 39.

In some embodiments of crystalline Formula I p-TSA salt Form I, at least one, at least two, or all of the following (a)-(c) apply: (a) crystalline Formula I p-TSA salt Form I has an XRPD pattern substantially as shown in FIG. 37; (b) crystalline Formula I p-TSA salt Form I has a DSC thermogram substantially as shown in FIG. 38; (c) crystalline Formula I p-TSA salt Form I has a TGA thermogram substantially as shown in FIG. 39.

In some embodiments, crystalline Formula I p-TSA salt Form I has at least one, at least two, or at least three of the following properties:
(a) an XRPD pattern substantially as shown in FIG. 37
(b) a DSC thermogram substantially as shown in FIG. 38
(c) a TGA thermogram substantially as shown in FIG. 39.

In some embodiments, crystalline Formula I p-TSA salt Form I has an XRPD pattern displaying at least two, at least three, at least four, at least five, at least six, at least seven, at least eight, or at least nine of the degree 2θ-reflections with the greatest intensity as the XRPD pattern substantially as shown in FIG. 37.

In certain embodiments, crystalline Formula I p-TSA salt Form I has an XRPD pattern comprising degree 2θ-reflections (±0.2 degrees 2θ) at 16.6, 20.9, 22.3, and 23.4 degrees.

In some embodiments, Formula I p-TSA salt Form I has an XRPD pattern comprising degree 2θ-reflections (±0.2 degrees 2θ) at 16.6, 20.9, 22.3, and 23.4 degrees and one, two, or three of the degree 2θ-reflections (±0.2 degrees 2θ) at 3.0, 13.1, and 21.5 degrees.

In some embodiments, Formula I p-TSA salt Form I has an XRPD pattern comprising degree 2θ-reflections (±0.2 degrees 2θ) at 16.6, 20.9, 22.3, and 23.4 degrees and one, two, or three of the degree 2θ-reflections (±0.2 degrees 2θ) at 9.0, 15.0, 17.5, and 26 degrees.

In some embodiments, Formula I p-TSA salt Form I has an XRPD pattern comprising degree 2θ-reflections (±0.2 degrees 2θ) at 16.6, 20.9, 22.3, 23.4, 3.0, 13.1, 21.5, 9.0, 15.0, 17.5, and 26 degrees.

In some embodiments, Formula I p-TSA salt Form I has a differential scanning calorimetry thermogram having an endotherm with onset at about 198° C. In some embodiments, Formula I p-TSA salt Form I has a differential scanning calorimetry thermogram having an exotherm with onset at about 208° C.

Formula I p-Toluenesulfonic Acid (p-TSA) Salt Form II

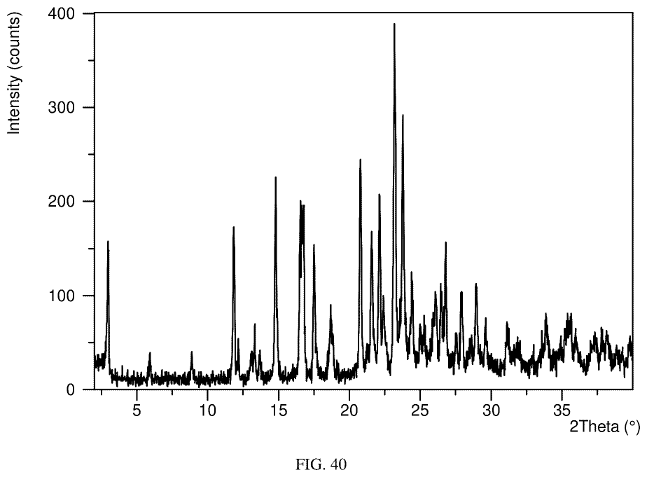

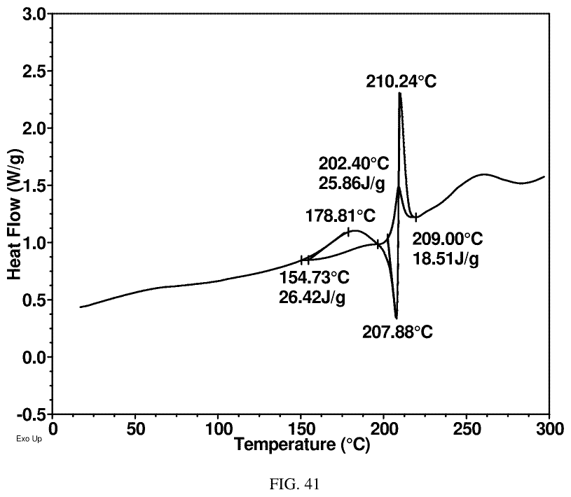

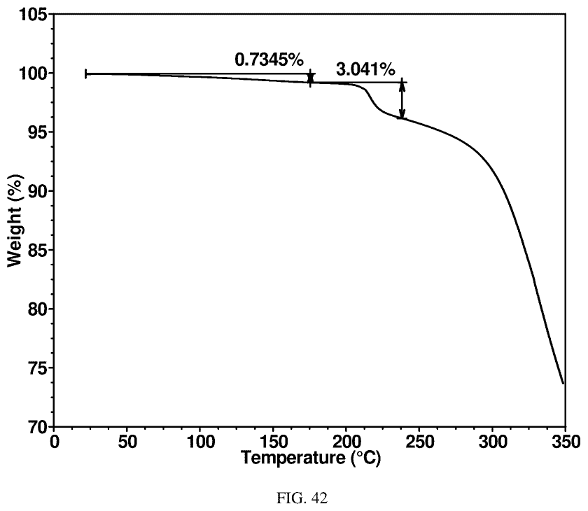

In some embodiments, provided herein is a crystalline compound of Formula I p-TSA salt Form II (Formula I p-TSA salt Form II), wherein the crystal structure exhibits an X-ray powder diffraction (XRPD) pattern substantially as shown in FIG. 40. Formula I p-TSA salt Form II may exhibit a differential scanning calorimetry (DSC) thermogram substantially as shown in FIG. 41. Crystalline Formula I p-TSA salt Form II may exhibit a thermogravimetric analysis (TGA) thermogram substantially as shown in FIG. 42.

In some embodiments of crystalline Formula I p-TSA salt Form II, at least one, at least two, or all of the following (a)-(c) apply: (a) crystalline Formula I p-TSA salt Form II has an XRPD pattern substantially as shown in FIG. 40; (b) crystalline Formula I p-TSA salt Form II has a DSC thermogram substantially as shown in FIG. 41; (c) crystalline Formula I p-TSA salt Form II has a TGA thermogram substantially as shown in FIG. 42.

In some embodiments, crystalline Formula I p-TSA salt Form II has at least one, at least two, or at least three of the following properties:
(a) an XRPD pattern substantially as shown in FIG. 40
(b) a DSC thermogram substantially as shown in FIG. 41
(c) a TGA thermogram substantially as shown in FIG. 42.

In some embodiments, crystalline Formula I p-TSA salt Form II has an XRPD pattern displaying at least two, at least three, at least four, at least five, at least six, at least seven, at least eight, or at least nine of the degree 2θ-reflections with the greatest intensity as the XRPD pattern substantially as shown in FIG. 40.

In certain embodiments, crystalline Formula I p-TSA salt Form II has an XRPD pattern comprising degree 2θ-reflections (±0.2 degrees 2θ) at 14.8, 20.8, 23.2, and 23.8 degrees.

In some embodiments, Formula I p-TSA salt Form II has an XRPD pattern comprising degree 2θ-reflections (±0.2 degrees 2θ) at 14.8, 20.8, 23.2, and 23.8 degrees and one, two, or three of the degree 2θ-reflections (±0.2 degrees 2θ) at 21.6, 22.1, and 24.4 degrees.

In some embodiments, Formula I p-TSA salt Form II has an XRPD pattern comprising degree 2θ-reflections (±0.2 degrees 2θ) at 14.8, 20.8, 23.2, and 23.8 degrees and one, two, or three of the degree 2θ-reflections (±0.2 degrees 2θ) at 3.0, 11.8, and 16.8 degrees.

In some embodiments, Formula I p-TSA salt Form I has an XRPD pattern comprising degree 2θ-reflections (±0.2 degrees 2θ) at 14.8, 20.8, 23.2, 23.8, 21.6, 22.1, 24.4, 3.0, 11.8, and 16.8 degrees.

In some embodiments, Formula I p-TSA salt Form II has a differential scanning calorimetry thermogram having an endotherm with onset at about 202° C. In some embodiments, Formula I p-TSA salt Form II has a differential scanning calorimetry thermogram having an exotherm with onset at about 155° C. In some embodiments, Formula I p-TSA salt Form II has a differential scanning calorimetry thermogram having an exotherm onset at about 209° C.

Formula I p-Toluenesulfonic Acid (p-TSA) Salt Form III

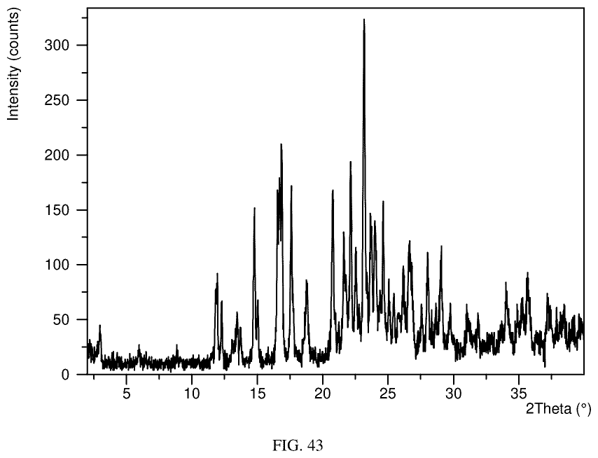

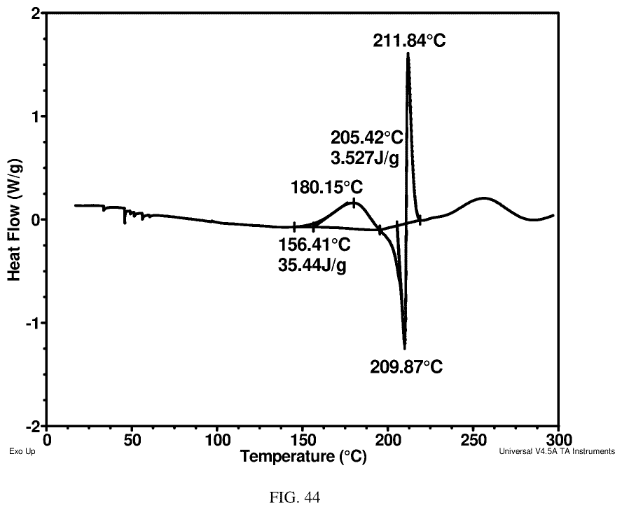

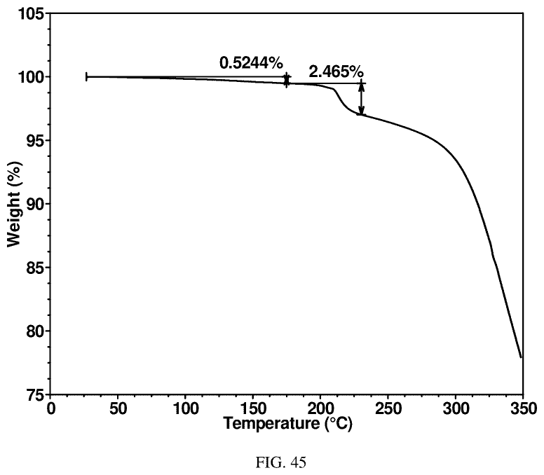

In some embodiments, provided herein is a crystalline compound of Formula I p-TSA salt Form III (Formula I p-TSA salt Form I), wherein the crystal structure exhibits an X-ray powder diffraction (XRPD) pattern substantially as shown in FIG. 43. Formula I p-TSA salt Form III may exhibit a differential scanning calorimetry (DSC) thermogram substantially as shown in FIG. 44. Crystalline Formula I p-TSA salt Form III may exhibit a thermogravimetric analysis (TGA) thermogram substantially as shown in FIG. 45.

In some embodiments of crystalline Formula I p-TSA salt Form III, at least one, at least two, or all of the following (a)-(c) apply: (a) crystalline Formula I p-TSA salt Form III has an XRPD pattern substantially as shown in FIG. 43; (b) crystalline Formula I p-TSA salt Form III has a DSC thermogram substantially as shown in FIG. 44; (c) crystalline Formula I p-TSA salt Form III has a TGA thermogram substantially as shown in FIG. 45.

In some embodiments, crystalline Formula I p-TSA salt Form III has at least one, at least two, or at least three of the following properties:
(a) an XRPD pattern substantially as shown in FIG. 43
(b) a DSC thermogram substantially as shown in FIG. 44
(c) a TGA thermogram substantially as shown in FIG. 45.

In some embodiments, crystalline Formula I p-TSA salt Form III has an XRPD pattern displaying at least two, at least three, at least four, at least five, at least six, at least seven, at least eight, or at least nine of the degree 2θ-reflections with the greatest intensity as the XRPD pattern substantially as shown in FIG. 43.

In certain embodiments, crystalline Formula I p-TSA salt Form III has an XRPD pattern comprising degree 2θ-reflections (±0.2 degrees 2θ) at 11.9, 14.8, 16.9, and 23.2 degrees.

In some embodiments, Formula I p-TSA salt Form III has an XRPD pattern comprising degree 2θ-reflections (±0.2 degrees 2θ) at 11.9, 14.8, 16.9, and 23.2 degrees and one, two, or three of the degree 2θ-reflections (±0.2 degrees 2θ) at 17.6, 20.8, 22.1, and 24.6 degrees.

In some embodiments, Formula I p-TSA salt Form III has an XRPD pattern comprising degree 2θ-reflections (±0.2 degrees 2θ) at 11.9, 14.8, 16.9, and 23.2 degrees and one, two, or three of the degree 2θ-reflections (±0.2 degrees 2θ) at 12.3, 18.8, and 26.6 degrees.

In some embodiments, Formula I p-TSA salt Form I has an XRPD pattern comprising degree 2θ-reflections (±0.2 degrees 2θ) at 11.9, 14.8, 16.9, 23.2, 17.6, 20.8, 22.1, 24.6, 12.3, 18.8, and 26.6 degrees.

In some embodiments, Formula I p-TSA salt Form III has a differential scanning calorimetry thermogram having an exotherm with onset at about 156° C. In some embodiments, Formula I p-TSA salt Form III has a differential scanning calorimetry thermogram having endotherm with onset at about 205° C.

Formula I p-Toluenesulfonic Acid (p-TSA) Salt Hydrate

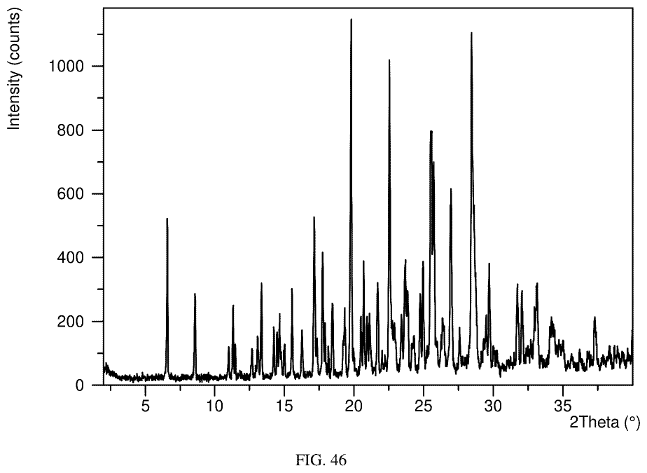

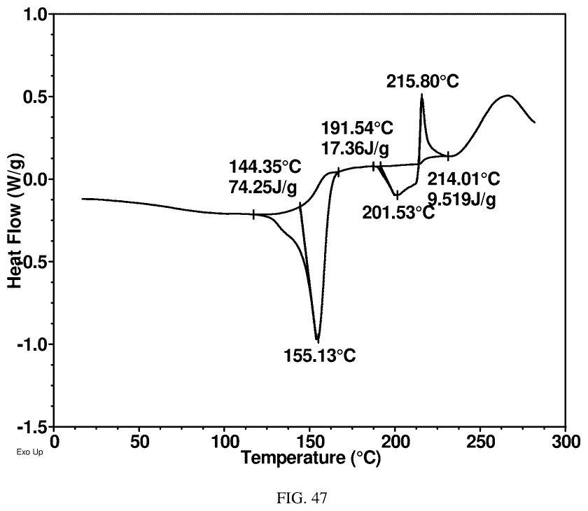

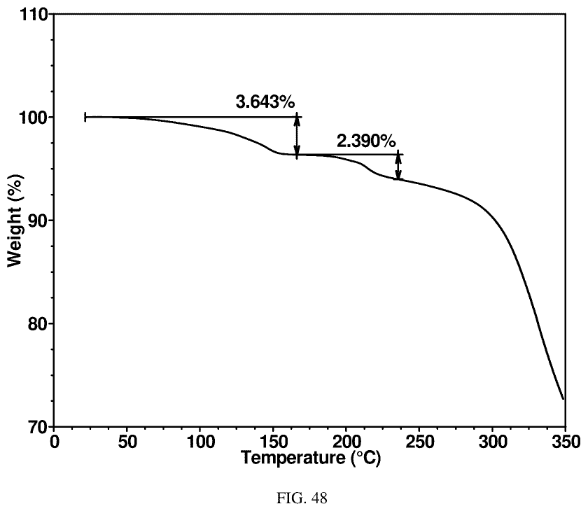

In some embodiments, provided herein is a crystalline compound of Formula I p-TSA salt Form I (Formula I p-TSA salt hydrate), wherein the crystal structure exhibits an X-ray powder diffraction (XRPD) pattern substantially as shown in FIG. 46. Formula I p-TSA salt hydrate may exhibit a differential scanning calorimetry (DSC) thermogram substantially as shown in FIG. 47. Crystalline Formula I p-TSA salt hydrate may exhibit a thermogravimetric analysis (TGA) thermogram substantially as shown in FIG. 48.

In some embodiments of crystalline Formula I p-TSA salt hydrate, at least one, at least two, or all of the following (a)-(c) apply: (a) crystalline Formula I p-TSA salt hydrate has an XRPD pattern substantially as shown in FIG. 46; (b) crystalline Formula I p-TSA salt hydrate has a DSC thermogram substantially as shown in FIG. 47; (c) crystalline Formula I p-TSA salt hydrate has a TGA thermogram substantially as shown in FIG. 48.

In some embodiments, crystalline Formula I p-TSA salt hydrate has at least one, at least two, or at least three of the following properties:
(a) an XRPD pattern substantially as shown in FIG. 46
(b) a DSC thermogram substantially as shown in FIG. 47
(c) a TGA thermogram substantially as shown in FIG. 48.

In some embodiments, crystalline Formula I p-TSA salt hydrate has an XRPD pattern displaying at least two, at least three, at least four, at least five, at least six, at least seven, at least eight, or at least nine of the degree 2θ-reflections with the greatest intensity as the XRPD pattern substantially as shown in FIG. 46.

In certain embodiments, crystalline Formula I p-TSA salt hydrate has an XRPD pattern comprising degree 2θ-reflections (±0.2 degrees 2θ) at 6.6, 8.6, 19.8, and 25.5 degrees.

In some embodiments, Formula I p-TSA salt hydrate has an XRPD pattern comprising degree 2θ-reflections (±0.2 degrees 2θ) at 6.6, 8.6, 19.8, and 25.5 degrees and one, two, three, or four of the degree 2θ-reflections (±0.2 degrees 2θ) at 11.3, 17.1, 22.5, and 28.4 degrees.

In some embodiments, Formula I p-TSA salt hydrate has an XRPD pattern comprising degree 2θ-reflections (±0.2 degrees 2θ) at 6.6, 8.6, 19.8, and 25.5 degrees and one, two, or three of the degree 2θ-reflections (±0.2 degrees 2θ) 15.6, 17.7, and 26.9 degrees.

In some embodiments, Formula I p-TSA salt hydrate has an XRPD pattern comprising degree 2θ-reflections (±0.2 degrees 2θ) at 6.6, 8.6, 19.8, 25.5, 11.3, 17.1, 22.5, and 28.4, 15.6, 17.7, and 26.9 degrees.

In some embodiments, Formula I p-TSA salt hydrate has a differential scanning calorimetry thermogram having an endotherm with onset at about 144° C. In some embodiments, Formula I p-TSA salt hydrate has a differential scanning calorimetry thermogram having endotherm with onset at about 192° C. In some embodiments, Formula I p-TSA salt hydrate has a differential scanning calorimetry thermogram having an exotherm with onset at about 214° C.

### Pharmaceutical Compositions

For the purposes of administration, in certain embodiments, the compounds described herein are administered as a raw chemical or are formulated as pharmaceutical compositions. Pharmaceutical compositions of the present disclosure can include a therapeutically effective amount of a compound of Formula I, and at least one pharmaceutically acceptable carrier and/or excipient. The compound of Formula I is present in the composition in an amount which is effective to treat a particular disease or condition of interest. The pharmaceutical compositions of the present disclosure may additionally comprise one or more other compounds as active ingredients, including for instance prodrugs, other nuclear receptor modulators, or other active pharmaceutical ingredients such as active pharmaceutical ingredients for use in treating liver disease, such as ACC inhibitors or ASK1 inhibitors. In some embodiments, the pharmaceutical compositions of the present disclosure additionally comprise an ACC inhibitor and an ASK1 inhibitor.

In some embodiments, a pharmaceutical composition includes Formula I zwitterion Form I; Formula I zwitterion Form II; Formula I hydrate; Formula I zwitterion amorphous; Formula I tromethamine salt Form I; Formula I tromethamine salt Form II; Formula I tromethamine salt hydrate I; Formula I tromethamine salt hydrate II; Formula I tromethamine salt hydrate III; Formula I tromethamine salt hydrate IV; Formula I tromethamine salt methanol solvate I; Formula I tromethamine salt methanol solvate II; Formula I tromethamine salt methanol solvate III; Formula I tromethamine salt MTBE solvate; Formula I tromethamine amorphous form; Formula I tromethamine salt ethanol solvate; Formula I p-toluenesulfonic acid salt Form I; Formula I p-toluenesulfonic acid salt Form II; Formula I p-toluenesulfonic acid salt Form III; and/or Formula I p-toluenesulfonic acid salt hydrate. In some embodiments, a pharmaceutical composition includes Formula I zwitterion Form I; Formula I zwitterion Form II; Formula I hydrate; Formula I zwitterion amorphous; Formula I tromethamine salt Form I; Formula I tromethamine salt Form II; Formula I tromethamine salt hydrate I; Formula I tromethamine salt hydrate II; Formula I tromethamine salt hydrate III; Formula I tromethamine salt hydrate IV; Formula I tromethamine salt methanol solvate I; Formula I tromethamine salt methanol solvate II; Formula I tromethamine salt methanol solvate III; Formula I tromethamine salt MTBE solvate; Formula I tromethamine amorphous form; Formula I tromethamine salt ethanol solvate; Formula I p-toluenesulfonic acid salt Form I; Formula I p-toluenesulfonic acid salt Form II; Formula I p-toluenesulfonic acid salt Form III; and/or Formula I p-toluenesulfonic acid salt hydrate and a pharmaceutically acceptable carrier.

In some embodiments, a pharmaceutical composition comprises a compound of Formula I (or zwitterion, hydrate, salt or solvate thereof), wherein at least 95% of Formula I is zwitterion Form I, and no more than 5%, 4%, 3%, 2% or 1% of other forms. In some embodiments, a pharmaceutical composition comprises a compound of Formula I (or zwitterion, hydrate, salt or solvate thereof), wherein at least 95% of Formula I is zwitterion Form II, and no more than 5%, 4%, 3%, 2% or 1% of other forms. In some embodiments, a pharmaceutical composition comprises a compound of Formula I (or zwitterion, hydrate, salt or solvate thereof), wherein at least 95% of Formula I is a hydrate, and no more than 5%, 4%, 3%, 2% or 1% of other forms. In some embodiments, a pharmaceutical composition comprises a compound of Formula I (or zwitterion, hydrate, salt or solvate thereof), wherein at least 95% of Formula I is Formula I zwitterion amorphous, and no more than 5%, 4%, 3%, 2% or 1% of other forms. In some embodiments, a pharmaceutical composition comprises a compound of Formula I (or zwitterion, hydrate, salt or solvate thereof), wherein at least 95% of Formula I is Formula I tromethamine salt Form I, and no more than 5%, 4%, 3%, 2% or 1% of other forms. In some embodiments, a pharmaceutical composition comprises a compound of Formula I (or zwitterion, hydrate, salt or solvate thereof), wherein at least 95% of Formula I is Formula I tromethamine salt Form II, and no more than 5%, 4%, 3%, 2% or 1% of other forms. In some embodiments, a pharmaceutical composition comprises a compound of Formula I (or zwitterion, hydrate, salt or solvate thereof), wherein at least 95% of Formula I is Formula I tromethamine salt hydrate I, and no more than 5%, 4%, 3%, 2% or 1% of other forms. In some embodiments, a pharmaceutical composition comprises a compound of Formula I (or zwitterion, hydrate, salt or solvate thereof), wherein at least 95% of Formula I is Formula I tromethamine salt hydrate II, and no more than 5%, 4%, 3%, 2% or 1% of other forms. In some embodiments, a pharmaceutical composition comprises a compound of Formula I (or zwitterion, hydrate, salt or solvate thereof), wherein at least 95% of Formula I is Formula I tromethamine salt hydrate III, and no more than 5%, 4%, 3%, 2% or 1% of other forms. In some embodiments, a pharmaceutical composition comprises a compound of Formula I (or zwitterion, hydrate, salt or solvate thereof), wherein at least 95% of Formula I is Formula I tromethamine salt hydrate IV, and no more than 5%, 4%, 3%, 2% or 1% of other forms. In some embodiments, a pharmaceutical composition comprises a compound of Formula I (or zwitterion, hydrate, salt or solvate thereof), wherein at least 95% of Formula I is Formula I tromethamine salt methanol solvate I, and no more than 5%, 4%, 3%, 2% or 1% of other forms. In some embodiments, a pharmaceutical composition comprises a compound of Formula I (or zwitterion, hydrate, salt or solvate thereof), wherein at least 95% of Formula I is Formula I tromethamine salt methanol solvate II, and no more than 5%, 4%, 3%, 2% or 1% of other forms. In some embodiments, a pharmaceutical composition comprises a compound of Formula I (or zwitterion, hydrate, salt or solvate thereof), wherein at least 95% of Formula I is Formula I tromethamine salt methanol solvate III, and no more than 5%, 4%, 3%, 2% or 1% of other forms. In some embodiments, a pharmaceutical composition comprises a compound of Formula I (or zwitterion, hydrate, salt or solvate thereof), wherein at least 95% of Formula I is Formula I tromethamine salt MTBE solvate, and no more than 5%, 4%, 3%, 2% or 1% of other forms. In some embodiments, a pharmaceutical composition comprises a compound of Formula I (or zwitterion, hydrate, salt or solvate thereof), wherein at least 95% of Formula I is Formula I tromethamine amorphous form, and no more than 5%, 4%, 3%, 2% or 1% of other forms. In some embodiments, a pharmaceutical composition comprises a compound of Formula I (or zwitterion, hydrate, salt or solvate thereof), wherein at least 95% of Formula I is Formula I tromethamine salt ethanol solvate, and no more than 5%, 4%, 3%, 2% or 1% of other forms. In some embodiments, a pharmaceutical composition comprises a compound of Formula I (or zwitterion, hydrate, salt or solvate thereof), wherein at least 95% of Formula I is Formula I p-toluenesulfonic acid salt Form I, and no more than 5%, 4%, 3%, 2% or 1% of other forms. In some embodiments, a pharmaceutical composition comprises a compound of Formula I (or zwitterion, hydrate, salt or solvate thereof), wherein at least 95% of Formula I is Formula I p-toluenesulfonic acid salt Form II, and no more than 5%, 4%, 3%, 2% or 1% of other forms. In some embodiments, a pharmaceutical composition comprises a compound of Formula I (or zwitterion, hydrate, salt or solvate thereof), wherein at least 95% of Formula I is Formula I p-toluenesulfonic acid salt Form III, and no more than 5%, 4%, 3%, 2% or 1% of other forms. In some embodiments, a pharmaceutical composition comprises a compound of Formula I (or zwitterion, hydrate, salt or solvate thereof), wherein at least 95% of Formula I is Formula I p-toluenesulfonic acid salt hydrate, and no more than 5%, 4%, 3%, 2% or 1% of other forms. In any of such embodiments, the pharmaceutical composition comprises a pharmaceutically acceptable carrier.

The activity of compounds of Formula I can be determined by one skilled in the art, for example, as described herein. Appropriate therapeutically effective concentrations and dosages can be readily determined by one skilled in the art.

In certain embodiments, the crystalline, salt, and/or solvate forms described herein may potentially exhibit improved properties. For example, in certain embodiments, the crystalline and/or salt forms described herein may potentially exhibit improved stability. Such improved stability could have a potentially beneficial impact on the manufacture of the compound of Formula I, such as for example offering the ability to store process intermediate for extended periods of time. Improved stability could also potentially benefit a composition or pharmaceutical composition of the compound of Formula I. In certain embodiments, the crystalline salt, and/or solvate forms described herein may also potentially result in improved yield of the compound of Formula I, or in an improvement of the quality of the compound of Formula I. In certain embodiments, the crystalline, salt, and/or solvate forms described herein may also exhibit improved pharmacokinetic properties and/or potentially improved bioavailability.

The compositions are suitable for oral, rectal, topical, parenteral (including subcutaneous, intramuscular, and intravenous), ocular (ophthalmic), pulmonary (nasal or buccal inhalation) or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of the present disclosure can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are employed. If desired, tablets may be coated by standard aqueous or non-aqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate, microcrystalline cellulose, lactose monohydrate, mannitol or colloidal silicon dioxide; a disintegrating agent such as corn starch, potato starch, alginic acid, croscarmellose sodium or crospovidone; a lubricant such as magnesium stearate; and a sweetening agent such as sucrose, lactose or saccharin. When a dosage unit form is a capsule, it may contain, in addition to materials of the above type, a liquid carrier such as a fatty oil or pharmaceutical media, such as, for example, water, glycols (e.g., polyethylene glycol 400), or alcohols.

Various other materials may be present as coatings or to modify the physical form of the dosage unit. For instance, tablets may be coated with shellac, sugar, polyvinyl alcohol, polyethylene glycol 3350, titanium dioxide, talc, coloring agent, or combinations thereof. A syrup or elixir may contain, in addition to the active ingredient, sucrose as a sweetening agent, methyl and propylparabens as preservatives, a dye and a flavoring such as cherry or orange flavor.

The compounds of the present disclosure may also be administered parenterally. Solutions or suspensions of these active compounds can be prepared in water suitably mixed with an organic, an additive, or combinations thereof. Examples of organics include, but are not limited to, N-methyl pyrrolidone, dimethylsulfoxide, polyethylene glycols, and combinations thereof. Examples of additives include, but are not limited to, hydroxy-propylcellulose, polyvinylpyrrolidone, poloxamers, poly(lactic-co-glycolic acid), polysorbates, povidone, carboxymethylcellulose, and combinations thereof. Dispersions can also be prepared in glycerol, liquid polyethylene glycols and mixtures thereof in oils. Under ordinary conditions of storage and use, these preparations may contain a preservative to, for instance, prevent the growth of microorganisms. In some embodiments, the parenteral administration includes intravenous administration with formulations comprising solutions with a mixture of organics and aqueous media. In some embodiments, the intravenous administration is dosed as a 100% organic solution.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. For instance, the forms can be stable under the conditions of manufacture and storage. The forms can be preserved against the contaminating action of microorganisms such as bacteria and fungi (for instance, via use of preservatives). The carrier can be a solvent or dispersion medium containing, for example, water, ethanol, polyol (e.g., glycerol, propylene glycol and liquid polyethylene glycol), suitable mixtures thereof, and vegetable oils.

Any suitable route of administration may be employed for providing a mammal, especially a human, with an effective dose of a compound of the present disclosure. For example, oral, rectal, topical, parenteral, ocular, pulmonary, nasal, and the like may be employed. Dosage forms include tablets, troches, dispersions, suspensions, solutions, capsules, creams, ointments, aerosols, and the like. In some embodiments, compounds of the present disclosure are administered orally.

In some embodiments, the composition comprises a crystalline form of Formula I or a pharmaceutically acceptable salt thereof. In some embodiments, the composition includes a crystalline form of a zwitterion of the compound of Formula I. In some embodiments, the composition includes a crystalline form of a tromethamine salt of the compound of Formula I. In certain embodiments are provided compositions comprising a crystalline form as described herein, wherein the Formula I within the composition is substantially pure (i.e., substantially pure Formula I zwitterion Form I; Formula I zwitterion Form II; Formula I hydrate; Formula I zwitterion amorphous; Formula I tromethamine salt Form I; Formula I tromethamine salt Form II; Formula I tromethamine salt hydrate I; Formula I tromethamine salt hydrate II; Formula I tromethamine salt hydrate III; Formula I tromethamine salt hydrate IV; Formula I tromethamine salt methanol solvate I; Formula I tromethamine salt methanol solvate II; Formula I tromethamine salt methanol solvate III; Formula I tromethamine salt MTBE solvate; Formula I tromethamine amorphous form; Formula I tromethamine salt ethanol solvate; Formula I; Formula I p-toluenesulfonic acid salt Form I; Formula I p-toluenesulfonic acid salt Form II; Formula I p-toluenesulfonic acid salt Form III; or Formula I p-toluenesulfonic acid salt hydrate).

In particular embodiments of compositions comprising a crystalline form of Formula I or a pharmaceutically acceptable salt thereof, at least about 50%, at least about 60%, at least about 70%, at least about 80%, at least about 85%, at least about 90%, at least about 95%, at least about 96%, at least about 97%, at least about 98%, or at least about 99% of Formula I present in the composition is one of the crystalline forms disclosed herein. In certain embodiments, the composition includes at least about 50%, at least about 60%, at least about 70%, at least about 80%, at least about 85%, at least about 90%, at least about 95%, at least about 96%, at least about 97%, at least about 98%, or at least about 99% of one of the crystalline forms of Formula I.

In other embodiments of compositions comprising a crystalline form disclosed herein, less than about 50%, less than about 40%, less than about 30%, less than about 20%, less than about 10%, less than about 5%, less than about 4%, less than about 3%, less than about 2% or less than about 1% of Formula I present in the composition are other amorphous or crystal forms of Formula I and/or impurities.

In yet other embodiments of compositions comprising the crystalline forms disclosed herein, impurities make up less than about 5%, less than about 4%, less than about 3%, less than about 2% or less than about 1% of the total mass relative to the mass of the crystalline forms present. Impurities may, for example, include by-products from synthesizing Formula I, contaminants, degradation products, other crystalline forms, amorphous form, water, and solvents. In certain embodiments, impurities include by-products from the process of synthesizing Formula I. In certain embodiments, impurities include contaminants from the process of synthesizing Formula I. In certain embodiments, impurities include degradation products of Formula I. In certain embodiments, impurities include other crystalline forms of Formula I. In certain embodiments, impurities include other crystalline forms of Formula I and/or amorphous forms of Formula I. In certain embodiments, impurities include water or solvent. In certain embodiments of compositions comprising a crystalline form disclosed herein, impurities are selected from the group consisting of by-products from synthesizing Formula I, contaminants, degradation products, other crystalline forms, amorphous forms, water, solvents and combinations thereof.

### Dosage

The effective dosage of active ingredient employed may vary depending on the particular compound employed, the mode of administration, the condition being treated and the severity of the condition being treated. Such dosage may be ascertained readily by a person skilled in the art.

When treating or preventing FXR mediated conditions for which compounds of the present disclosure are indicated, generally satisfactory results are obtained when the compounds of the present disclosure are administered at a daily dosage of from about 0.1 milligram to about 100 milligram per kilogram of animal body weight. In some embodiments, the compounds of the present disclosure are given as a single daily dose or in divided doses two to six times a day, or in sustained release form. For most large mammals, the total daily dosage can be from about 1 milligram to about 1000 milligrams. In the case of a 70 kg adult human, the total daily dose will generally be from about 7 milligrams to about 350 milligrams. This dosage regimen may be adjusted to provide the optimal therapeutic response. In some embodiments, the total daily dosage is from about 1 milligram to about 900 milligrams, about 10 milligrams to about 800 milligrams, about 20 milligrams to about 700 milligrams, about 30 milligrams to about 600 milligrams, about 40 milligrams to about 550 milligrams, or about 50 milligrams to about 400 milligrams. In some embodiments, the total daily dosage is from about 10 milligrams to about 50 milligrams, from about 20 milligrams to about 40 milligrams, from about 25 milligrams to about 35 milligrams, from about 50 milligrams to about 150 milligrams, from about 70 milligrams to about 130 milligrams, from about 80 milligrams to about 120 milligrams, from about 90 milligrams to about 100 milligrams, from about 1 milligram to about 150 milligrams, from about 1 milligram to about 75 milligrams, from about 1 milligram to about 50 milligrams, from about 25 milligrams to about 125 milligrams, from about 125 milligrams to about 275 milligrams, from about 275 milligrams to about 425 milligrams, from about 425 milligrams to about 575 milligrams, from about 575 milligrams to about 725 milligrams, from about 725 milligrams to about 875 milligrams, or from about 875 milligrams to about 1000 milligrams.

The compounds of the present application or the compositions thereof may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment. Treatment cycles are well known in cancer chemotherapy, and are frequently alternated with resting periods of about 1 to 28 days, commonly about 7 days or about 14 days, between cycles. In some embodiments, the treatment cycle is 30 days. The treatment cycles, in other embodiments, may also be continuous.

In a particular embodiment, the methods provided herein comprise administering to the subject an initial daily dose of about 1 mg to about 800 mg of a compound described herein and increasing the dose by increments until clinical efficacy is achieved. Increments of about 5, 10, 25, 30, 40, 50, or 100 mg can be used to increase the dose. The dosage can be increased daily, every other day, twice per week, once per week or once every 4 weeks.

### Treatment Methods and Uses

“Treatment” or “treating” is an approach for obtaining beneficial or desired results including clinical results. Beneficial or desired clinical results may include one or more of the following: (a) inhibiting the disease or condition (e.g., decreasing one or more symptoms resulting from the disease or condition, and/or diminishing the extent of the disease or condition); (b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or (c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

The disclosure further relates to the use of said compounds for the treatment and/or prophylaxis of diseases and/or conditions through binding of said nuclear receptor by said compounds. Further the present disclosure relates to the use of said compounds for the preparation of a medicament for the treatment and/or prophylaxis of diseases and/or conditions through binding of said nuclear receptor by said compounds.

Also provided herein are methods of treating a patient having a FXR mediated condition. In some embodiments, the method includes administering a compound or composition disclosed herein. In some embodiments, a method of treating a patient having an FXR mediated condition includes administering a therapeutically effective amount of Formula I zwitterion Form I; Formula I zwitterion Form II; Formula I hydrate; Formula I zwitterion amorphous; Formula I tromethamine salt Form I; Formula I tromethamine salt Form II; Formula I tromethamine salt hydrate I; Formula I tromethamine salt hydrate II; Formula I tromethamine salt hydrate III; Formula I tromethamine salt hydrate IV; Formula I tromethamine salt methanol solvate I; Formula I tromethamine salt methanol solvate II; Formula I tromethamine salt methanol solvate III; Formula I tromethamine salt MTBE solvate; Formula I tromethamine amorphous form; Formula I tromethamine salt ethanol solvate; Formula I; Formula I p-toluenesulfonic acid salt Form I; Formula I p-toluenesulfonic acid salt Form II; Formula I p-toluenesulfonic acid salt Form III; and/or Formula I p-toluenesulfonic acid salt hydrate.

Also provided herein are methods of treating or preventing a disease or condition in a patient in need thereof, comprising administering a therapeutically effective amount of an FXR agonist, wherein the disease or condition is congenital hepatic fibrosis, and wherein the FXR agonist is a compound of Formula I

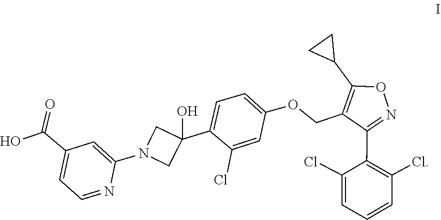 [SMILES: O=C(O)c1ccnc(N2CC(O)(c3ccc(OCc4c(-c5c(Cl)cccc5Cl)noc4C4CC4)cc3Cl)C2)c1]

In some embodiments, a method of treating a patient having congenital hepatic fibrosis includes administering a compound of Formula I. In some embodiments, a method of treating a patient having congenital hepatic fibrosis includes administering a solid form of a compound of Formula I.

In some embodiments a compound or composition disclosed herein is provided for use in the treatment of a FXR mediated condition.

In some embodiments, a compound or composition disclosed herein is provided for the manufacture of a medicament for the treatment of a FXR mediated condition.

In some embodiments, the FXR mediated condition is: a chronic intrahepatic or some form of extrahepatic cholestatic condition; liver fibrosis; an obstructive inflammatory disorder of the liver; chronic inflammatory disorder of the liver; liver cirrhosis; liver steatosis or an associated syndrome; cholestatic or fibrotic effects that are associated with alcohol-induced cirrhosis or with viral-borne forms of hepatitis; liver failure or liver ischemia after major liver resection; chemotherapy associated steatohepatitis (CASH); acute liver failure; or Inflammatory Bowel Disease.

In some embodiments, the FXR mediated condition is a lipid and lipoprotein disorder; Type I Diabetes; Type II Diabetes; clinical complications of Type I and Type II Diabetes selected from the group consisting of diabetic nephropathy, diabetic neuropathy, diabetic retinopathy and other observed effects of clinically manifest long term Diabetes; Non-Alcoholic Fatty Liver Disease (NAFLD); Non-Alcoholic Steatohepatitis (NASH); obesity; a metabolic syndrome selected from the group consisting of combined conditions of dyslipidemia, diabetes and abnormally high body-mass index; acute myocardial infarction; acute stroke; or thrombosis which occurs as an endpoint of chronic obstructive atherosclerosis.

In some embodiments, the FXR mediated condition is: a non-malignant hyperproliferative disorder; and a malignant hyperproliferative disorder selected from the group consisting of hepatocellular carcinoma, colon adenoma, and polyposis; colon adenocarcinoma; breast cancer; pancreas adenocarcinoma; Barrett's esophagus; or other forms of neoplastic diseases of the gastrointestinal tract and the liver.

In some embodiments, the FXR mediated condition is Non-Alcoholic Steatohepatitis (NASH), primary sclerosing cholangitis (PSC), or primary biliary cirrhosis (PBC).

In some embodiments, the FXR mediated condition is congenital hepatic fibrosis.

In some embodiments, the present disclosure relates to the use of compounds and compositions disclosed herein in the preparation of a medicament for the prophylaxis and/or treatment of chronic intrahepatic or some forms of extrahepatic cholestatic conditions, of liver fibrosis, of acute intrahepatic cholestatic conditions, of obstructive or chronic inflammatory disorders that arise out of improper bile composition, of gastrointestinal conditions with a reduced uptake of dietary fat and fat-soluble dietary vitamins, of inflammatory bowel diseases, of lipid and lipoprotein disorders, of Type II Diabetes and clinical complications of Type I and Type II Diabetes, of conditions and diseases which result from chronic fatty and fibrotic degeneration of organs due to enforced lipid and specifically triglyceride accumulation and subsequent activation of profibrotic pathways, of obesity and metabolic syndrome (combined conditions of dyslipidemia, diabetes and abnormally high body-mass index), of acute myocardial infarction, of acute stroke, of thrombosis which occurs as an endpoint of chronic obstructive atherosclerosis, of persistent infections by intracellular bacteria or parasitic protozoa, of non-malignant hyperproliferative disorders, of malignant hyperproliferative disorders, of colon adenocarcinoma and hepatocellular carcinoma in particular, of liver steatosis and associated syndromes, of liver failure or liver malfunction as an outcome of chronic liver diseases or of surgical liver resection, of Hepatitis B infection, of Hepatitis C infection, of cholestatic and fibrotic effects that are associated with alcohol-induced cirrhosis or with viral-borne forms of hepatitis, and/or of congenital hepatic fibrosis.

Medicaments as referred to herein may be prepared by conventional processes, including the combination of a compound according to the present disclosure and a pharmaceutically acceptable carrier.

### Kits

Provided herein are also kits that include a compound or composition described herein and suitable packaging. In one embodiment, a kit further includes instructions for use. In one aspect, a kit includes a crystalline form of the disclosure, or composition including a crystalline form of the disclosure and a label and/or instructions for use of the compounds in the treatment of the indications, including the diseases or conditions, described herein.

Provided herein are also articles of manufacture that include a compound or composition described herein in a suitable container. The container may be a vial, jar, ampoule, preloaded syringe, and intravenous bag.

### Combination Therapy

In some embodiments, disclosed herein are oral dosage forms (e.g., tablets) comprising a novel crystalline forms of a compound of Formula I:

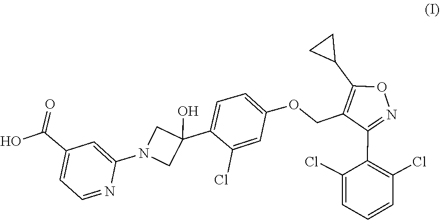 [SMILES: O=C(O)c1ccnc(N2CC(O)(c3ccc(OCc4c(-c5c(Cl)cccc5Cl)noc4C4CC4)cc3Cl)C2)c1]

or a pharmaceutically acceptable salt thereof and at least one additional therapeutic agent. In some embodiments, the oral dosage forms disclosed herein comprise novel crystal forms Formula I or a pharmaceutically acceptable salt thereof and one, two, or three additional therapeutic agents.

In some embodiments, the therapeutic agent, or combination of therapeutic agents, are a (n) ACE inhibitor, Acetaldehyde dehydrogenase inhibitor, Acetyl CoA carboxylase inhibitor, Acetyl COA carboxylase inhibitor, Diacylglycerol O acyltransferase 2 inhibitor, Adenosine A3 receptor agonist, Adiponectin receptor agonist, Aldehyde dehydrogenase 2 stimulator, AKT protein kinase inhibitor, AMP-activated protein kinases (AMPK), AMP kinase activator, ATP citrate lyase inhibitor, AMP activated protein kinase stimulator, Endothelial nitric oxide synthase stimulator, NAD-dependent deacetylase sirtuin-1 stimulator, Androgen receptor agonist, Amylin receptor agonist, Angiotensin II AT-1 receptor antagonist, Autophagy protein modulator, Autotaxin inhibitors, Axl tyrosine kinase receptor inhibitor, Bax protein stimulator, Bioactive lipid, Calcitonin agonist, Cannabinoid receptor modulator, Caspase inhibitor, Caspase-3 stimulator, Cathepsin inhibitor, Caveolin 1 inhibitor, CCR2 chemokine antagonist, CCR2 chemokine antagonist, Angiotensin II AT-1 receptor antagonist, CCR3 chemokine antagonist, CCR5 chemokine antagonist, CD3 antagonist, Chloride channel stimulator, CNR1 inhibitor, Cyclin D1 inhibitor, Cytochrome P450 7A1 inhibitor, DGAT1/2 inhibitor, Diacylglycerol O acyltransferase 1 inhibitor (DGAT1), Cytochrome P450 2E1 inhibitor (CYP2E1), CXCR4 chemokine antagonist, Dipeptidyl peptidase IV inhibitor, Endosialin modulator, Eotaxin ligand inhibitor, Extracellular matrix protein modulator, Farnesoid X receptor agonist, Fatty acid synthase inhibitors, FGF1 receptor agonist, Fibroblast growth factor (FGF-15, FGF-19, FGF-21) ligands, Galectin-3 inhibitor, Glucagon receptor agonist, Glucagon-like peptide 1 agonist, G-protein coupled bile acid receptor 1 agonist, G-protein coupled receptor 84 antagonist, Hedgehog (Hh) modulator, Hepatitis C virus NS3 protease inhibitor, Hepatocyte nuclear factor 4 alpha modulator (HNF4A), Hepatocyte growth factor modulator, Histone deacetylase inhibitor, STAT-3 modulator, HMG CoA reductase inhibitor, Hypoxia inducible factor-2 alpha inhibitor, IL-10 agonist, IL-17 antagonist, Ileal sodium bile acid cotransporter inhibitor, Insulin sensitizer, Insulin ligand agonist, Insulin receptor agonist, integrin modulator, Integrin Antagonist, intereukin-1 receptor-associated kinase 4 (IRAK4) inhibitor, IL-6 receptor agonist, Jak2 tyrosine kinase inhibitor, Ketohexokinase (KHK) inhibitor, Klotho beta stimulator, 5-Lipoxygenase inhibitor, Lipoprotein lipase inhibitor, Liver X receptor, LPL gene stimulator, Lysophosphatidate-1 receptor antagonist, Lysyl oxidase homolog 2 inhibitor, Macrophage mannose receptor 1 modulator, Matrix metalloproteinases (MMPs) inhibitor, MEKK-5 protein kinase inhibitor, MCH receptor-1 antagonist, Membrane copper amine oxidase (VAP-1) inhibitor, Methionine aminopeptidase-2 inhibitor, Methyl CpG binding protein 2 modulator, MicroRNA-21 (miR-21) inhibitor, Mitochondrial uncoupler, Mixed lineage kinase-3 inhibitor, Myelin basic protein stimulator, NACHT LRR PYD domain protein 3 (NLRP3) inhibitor, NAD-dependent deacetylase sirtuin stimulator, NADPH oxidase inhibitor (NOX), Nicotinic acid receptor 1 agonist, P2Y13 purinoceptor stimulator, Nuclear receptor modulators, P2X7 purinoceptor modulator, PDE 3 inhibitor, PDE 4 inhibitor, PDE 5 inhibitor, PDGF receptor beta modulator, Phenylalanine hydroxylase stimulator, Phospholipase C inhibitor, PPAR alpha agonist, PPAR delta agonist, PPAR gamma agonist, Peptidyl-prolyl cis-trans isomerase A inhibitor, PPAR gamma modulator, Protease-activated receptor-2 antagonist, Protein kinase modulator, Rho associated protein kinase inhibitor, Snitrosoglutathione reductase (GSNOR) enzyme inhibitor, Sodium glucose transporter-2 inhibitor, SREBP transcription factor inhibitor, STAT-1 inhibitor, Stearoyl CoA desaturase-1 inhibitor, STK25 inhibitor, Suppressor of cytokine signalling-1 stimulator, Suppressor of cytokine signalling-3 stimulator, Transforming growth factor β (TGF-β), Transforming growth factor β activated Kinase 1 (TAK1), Thyroid hormone receptor beta agonist, TLR-4 antagonist, Transglutaminase inhibitor, Tyrosine kinase receptor modulator, GPCR modulator, nuclear hormone receptor modulator, WNT modulators, or YAP/TAZ modulator and Zonulin inhibitor.

Non-limiting examples of the one or more additional therapeutic agents include:
ACE inhibitors, such as enalapril;
Acetaldehyde dehydrogenase inhibitors, such as ADX-629;
Acetyl CoA carboxylase (ACC) inhibitors, such as NDI-010976 (firsocostat), DRM-01, gemcabene, PF-05175157, QLT-091382, PF-0522 1304;
Acetyl CoA carboxylase/Diacylglycerol O acyltransferase 2 inhibitors, such as PF-07055341;
Adenosine receptor agonists, such as CF-102 (namodenoson), CF-101, CF-502, CGS21680;
Adiponectin receptor agonists, such as ADP-355, ADP-399;
Aldehyde dehydrogenase 2 stimulators, such as FP-045;
Amylin/calcitonin receptor agonists, such as KBP-042, KBP-089;
AMP activated protein kinase stimulators, such as, PXL-770, O-304;
AMP kinase activators/ATP citrate lyase inhibitors, such as as bempedoic acid (ETC-1002, ESP-55016)
AMP activated protein kinase/Endothelial nitric oxide synthase/NAD-dependent deacetylase sirtuin-1 stimulators, such as NS-0200;
Androgen receptor agonists, such as LPCN-1144;
Angiotensin II AT-1 receptor antagonists, such as irbesartan; Angiopoietin-related protein-3 inhibitors, such as IONIS-ANGPTL3-LRx;
Autophagy protein modulators, such as A-2906;
Autotaxin inhibitors, such as PAT-505, PAT-048, GLPG-1690, X-165, PF-8380, AM-063, BBT-877;
Axl tyrosine kinase receptor inhibitors, such as bemcentinib (BGB-324, R-428);
Bax protein stimulators, such as CBL-514;
Bioactive lipids, such as DS-102;
Cannabinoid receptor modulators, such as namacizumab, GWP-42004, REV-200, CRB-4001;
Caspase inhibitors, such as emricasan;
Pan cathepsin B inhibitors, such as VBY-376;
Pan cathepsin inhibitors, such as VBY-825;
CCR2/CCR5 chemokine antagonists, such as cenicriviroc, maraviroc, CCX-872, WXSH-0213;
CCR2 chemokine antagonists, such as propagermanium;
CCR2 chemokine/Angiotensin II AT-1 receptor antagonists, such as DMX-200, DMX-250;
CCR3 chemokine antagonists, such as bertilimumab;
CD3 antagonists, such as NI-0401;
Chloride channel stimulators, such as cobiprostone;
CXCR4 chemokine antagonists, such as AD-214;
Diglyceride acyltransferase 2 (DGAT2) inhibitors, such as IONIS-DGAT2Rx, PF-06865571;
Diglyceride acyltransferase 1 (DGAT1) inhibitors, such as GSK-3008356;
Diacylglycerol O acyltransferase 1 (DGAT1)/Cytochrome P450 2E1 inhibitors (CYP2E1), such as SNP-610;
Dipeptidyl peptidase IV inhibitors, such as linagliptin,evogliptin;
Eotaxin ligand inhibitors, such as bertilimumab, CM-101;
Extracellular matrix protein modulators, such as CNX-024;
Farnesoid X receptor (FXR) agonists, such as AGN-242266, AGN-242256, EP-024297, RDX-023, BWL-200, AKN-083, EDP-305, GNF-5120, GS-9674, LMB-763, obeticholic acid, Px-102, Px-103, M790, M780, M450, M-480, (MET-409), PX20606, EYP-001, TERN-101, TC-100, INT-2228;
Farnesoid X receptor (FXR)/G-protein coupled bile acid receptor 1 (TGR5) agonists, such as INT-767;
Fatty acid synthase inhibitors, such as TVB-2640;
Fibroblast growth factor 19 (rhFGF19)/cytochrome P450 (CYP) 7A1 inhibitors, such as NGM-282;
Fibroblast growth factor 21 (FGF-21) ligand, such as BMS-986171, BIO89-100,
BMS-986036, B-1344;
Fibroblast growth factor 21 (FGF-21)/glucagon like peptide 1 (GLP-1) agonist, such as YH-25723 AKR-001;
Galectin-3 inhibitors, such as GR-MD-02, GB-1107;
Glucagon-like peptide 1 (GLP1R) agonists, such as AC-3174, liraglutide, cotadutide (MEDI-0382), SAR-425899, LY-3305677, HM-15211, YH-25723, YH-GLP1, RPC-8844, PB-718, semaglutide;
G-protein coupled bile acid receptor 1 (TGR5) agonists, such as RDX-009, INT-777;
Heat shock protein 47 (HSP47) inhibitors, such as ND-L02-s0201;
Histone deacetylase inhibitors/STAT-3 modulators, such as SFX-01;
HMG CoA reductase inhibitors, such as atorvastatin, fluvastatin, pitavastatin, pravastatin, rosuvastatin, simvastatin;
Hypoxia inducible factor-2 alpha inhibitors, such as PT-2567;
IL-10 agonists, such as peg-ilodecakin;
Ileal sodium bile acid cotransporter inhibitors, such as odevixibat (A-4250), volixibat potassium ethanolate hydrate (SHP-262), GSK2330672, CJ-14199, elobixibat (A-3309);
Insulin sensitizers, such as, KBP-042, MSDC-0602K, MSDC-5514, Px-102, RG-125 (AZD4076), VVP-100X, CB-4211, ETI-101;
Insulin ligand/dsInsulin receptor agonists, such as ORMD-0801;
Integrin antagonists, such as IDL-2965;
IL-6 receptor agonists, such as KM-2702;
Ketohexokinase (KHK) inhibitors, such as PF-06835919;
beta Klotho (KLB)-FGF1c agonists, such as MK-3655 (NGM-313);
5-Lipoxygenase inhibitors, such as tipelukast (MN-001), DS-102 (AF-102);
Lipoprotein lipase inhibitors, such as CAT-2003;
LPL gene stimulators, such as alipogene tiparvovec;
Liver X receptor (LXR) inhibitors, such as PX-L603, PX-L493, BMS-852927, T-0901317, GW-3965, SR-9238;
Lysophosphatidate-1 receptor antagonists, such as BMT-053011, UD-009 (CP-2090), AR-479, ITMN-10534, BMS-986020, KI-16198;
Lysyl oxidase homolog 2 inhibitors, such as simtuzumab, PXS-5382A (PXS-5338);
Macrophage mannose receptor 1 modulators, such as tilmanocept-Cy3 (technetium Tc 99m tilmanocept);
Membrane copper amine oxidase (VAP-1) inhibitors, such as TERN-201;
MEKK-5 protein kinase (ASK-1) inhibitors, such as GS-4997, SRT-015, GS-444217, GST-HG-151;
MCH receptor-1 antagonists, such as CSTI-100 (ALB-127158);
Semicarbazide-Sensitive Amine Oxidase/Vascular Adhesion Protein-1 (SSAO/VAP-1) Inhibitors, such as PXS-4728A;
Methionine aminopeptidase-2 inhibitors, such as ZGN-1061, ZGN-839, ZN-1345;
Methyl CpG binding protein 2 modulators, such as mercaptamine;
Mineralocorticoid receptor antagonists (MCRA), such as MT-3995;
Mitochondrial uncouplers, such as 2,4-dinitrophenol;
Mixed lineage kinase-3 inhibitors, such as URMC-099-C;
Myelin basic protein stimulators, such as olesoxime;
Myeloperoxidase inhibitors, such as PF-06667272, AZM-198;
NADPH oxidase inhibitors, such as GKT-831, APX-311;
Nicotinic acid receptor 1 agonists, such as ARI-3037MO;
NACHT LRR PYD domain protein 3 (NLRP3) inhibitors, such as KDDF-201406-03, NBC-6, IFM-514, JT-194 (JT-349);
Nuclear receptor modulators, such as DUR-928 (DV-928);
P2X7 purinoceptor modulators, such as SGM-1019;
P2Y13 purinoceptor stimulators, such as CER-209;
PDE 3/4 inhibitors, such as tipelukast (MN-001);
PDE 5 inhibitors, such as sildenafil, MSTM-102;
PDGF receptor beta modulators, such as BOT-191, BOT-509;
Peptidyl-prolyl cis-trans isomerase inhibitors, such as CRV-431 (CPI-432-32), NVP-018, NV-556 (NVP-025);
Phenylalanine hydroxylase stimulators, such as HepaStem;
PPAR agonists, such as elafibranor (GFT-505), seladelpar lysine (MBX-8025), deuterated pioglitazone R-enantiomer, pioglitazone, DRX-065, saroglitazar, lanifibranor (IVA-337), CHS-131;
Protease-activated receptor-2 antagonists, such as PZ-235;
Protein kinase modulators, such as CNX-014;
Rho associated protein kinase (ROCK) inhibitors, such as REDX-10178 (REDX-10325), KD-025;
Snitrosoglutathione reductase (GSNOR) enzyme inhibitors, such as SL-891;
Sodium glucose transporter-2 (SGLT2) inhibitors, such as ipragliflozin, remogliflozin etabonate, ertugliflozin, dapagliflozin, tofogliflozin, sotagliflozin,
Sodium glucose transporter-1/2 (SGLT 1/2) inhibitors, such as licogliflozin bis(prolinate);
SREBP transcription factor inhibitors, such as CAT-2003, MDV-4463;
Stearoyl CoA desaturase-1 inhibitors, such as aramchol;
Thyroid hormone receptor beta agonists, such as resmetirom (MGL-3196), MGL-3745, VK-2809;
TLR-2/TLR-4 antagonists, such as VB-201 (CI-201);
TLR-4 antagonists, such as JKB-121;
Tyrosine kinase receptor modulators, such as CNX-025;
GPCR modulators, such as CNX-023;
Nuclear hormone receptor modulators, such as Px-102;
Xanthine oxidase/Urate anion exchanger 1 (URAT1) inhibitors, such as RLBN-1001, RLBN-1127; and
Zonulin Inhibitors, such as lorazotide acetate (INN-202).

In certain specific embodiments, the one or more additional therapeutic agents are selected from A-4250, AC-3174, acetylsalicylic acid, AK-20, alipogene tiparvovec, AMX-342, AN-3015, aramchol, ARI-3037MO, ASP-8232, AZD-2693, bertilimumab, Betaine anhydrous, BI-1467335, BMS-986036, BMS-986171, BMT-053011, BOT-191, BTT-1023, CAT-2003, cenicriviroc, CBW-511, CER-209, CF-102, CGS21680, CNX-014, CNX-023, CNX-024, CNX-025, cobiprostone, colesevelam, dapagliflozin, DCR-LIV1, deuterated pioglitazone R-enantiomer, 2,4-dinitrophenol, DRX-065, DS-102, DUR-928, EDP-305, elafibranor (GFT-505), emricasan, enalapril, ertugliflozin, evogliptin, F-351, fluasterone (ST-002), FT-4101, GKT-831, GNF-5120, GRI-0621, GR-MD-02, GS-300, GS-4997, GS-9674, HTD-1801, HST-202, HST-201, hydrochlorothiazide, icosabutate (PRC-4016), icosapent ethyl ester, IMM-124-E, INT-767, INV-240, IONIS-DGAT2Rx, ipragliflozin, Irbesarta, propagermanium, IVA-337, JKB-121, KB-GE-001, KBP-042, KD-025, M790, M780, M450, metformin, sildenafil, LC-280126, linagliptin, liraglutide, LJN-452, LM-011, LM-002 (CVI-LM-002), LMB-763, LYN-100, MBX-8025, MDV-4463, mercaptamine, MGL-3196, MGL-3745, MP-301, MSDC-0602K, namacizumab, NC-101, NDI-010976, ND-L02-s0201, NGM-282, NGM-313, NGM-386, NGM-395, NP-160, norursodeoxycholic acid, NVP-022, 0-304, obeticholic acid, 25HC3S, olesoxime, PAT-505, PAT-048, PB-4547, peg-ilodecakin, pioglitazone, pirfenidone, PRI-724, PX20606, Px-102, PX-L603, PX-L493, PXS-4728A, PZ-235, RDX-009, remogliflozin etabonate, RG-125 (AZD4076), RPI-500, saroglitazar, semaglutide, simtuzumab, solithromycin, sotagliflozin, statins (atorvastatin, fluvastatin, pitavastatin, pravastatin, rosuvastatin, simvastatin), TCM-606F, TEV-45478, TQA-3526, tipelukast (MN-001), TLY-012, TRX-318, TVB-2640, UD-009, ursodeoxycholic acid, VBY-376, VBY-825, VK-2809, vismodegib, volixibat potassium ethanolate hydrate (SHP-626), VVP-100X, WAV-301, WNT-974, XRx-117, ZGN-839, ZG-5216, ZSYM-008, ZYSM-007.

In some embodiments, methods and compositions include a therapeutically effective amount of an Apoptosis Signal-Regulating Kinase 1 (ASK1) inhibitor and a therapeutically effective amount of a Farnesoid X Receptor (FXR) agonist, wherein the FXR agonist is a solid form described herein.

In certain embodiments of the methods and pharmaceutical compositions disclosed herein, the ASK1 inhibitor is a compound of Formula II:

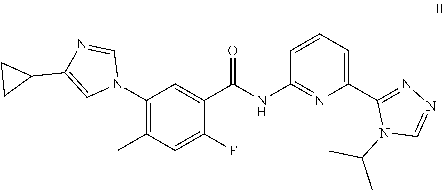 [SMILES: Cc1cc(F)c(C(=O)Nc2cccc(-c3nncn3C(C)C)n2)cc1-n1cnc(C2CC2)c1]

or a pharmaceutically acceptable salt, a stereoisomer, a mixture of stereoisomers, or a tautomer thereof.

ASK1 inhibitors, such as the compound of Formula (II), can be synthesized and characterized using methods known to those of skill in the art, such as those described in U.S. 2007/0276050 U.S. 2011/0009410, and U.S. 2013/0197037.

In some embodiments, methods and compositions include a therapeutically effective amount of an Acetyl COA Carboxylase inhibitor and a therapeutically effective amount of a Farnesoid X Receptor (FXR) agonist, wherein the FXR agonist is a solid form described herein.

### EXAMPLES

### Methods

Compounds of Formula I were synthesized according to known methods, such as those disclosed in U.S. Pat. No. 9,139,539.

### X-Ray Powder Diffraction (XRPD)

XRPD patterns were collected on a PANanalytical XPERT-PRO diffractometer at ambient conditions under the following experimental settings: 45 KV, 40 mA, Kα1=1.5406 Å, scan range 2 to 40° 2θ, step size 0.0084 or 0.0167° 2θ, measurement time: 5 min.

Some X-ray powder diffraction data were collected under ambient conditions on a Rigaku Miniflex 600 diffractometer using Cu K alpha (1.5406 Angstrom) radiation. Powder patterns were collected on a zero background holder with a 0.1 mm indent at a scan rate of 2 to 40° 2θ two theta at 2° 2θ per min at 40 kV and 15 mA.

Differential Scanning calorimetry (DSC)

DSC thermograms were collected on a TA Instruments Q2000 system equipped with a 50 position auto-sampler. The calibration for energy and temperature was carried out using certified indium. Typically 1-5 mg of each sample, in a pin-holed aluminum pan, was heated at 10° C./min from 25° C. to 300° C. A purge of dry nitrogen at 50 mL/min was maintained over the sample throughout the measurement. The onset of the melting endotherm was reported as the melting point.

Some DSC analyses were performed with a TA Discovery series DSC using a few milligrams of material in a Tzero aluminum pan sealed with a Tzero hermetic lid containing two pin holes. Samples were scanned at 10° C. per minute under 50 mL per minute of nitrogen flow.

### Thermo-Gravimetric Analysis (TGA)

TGA thermograms were collected on a TA Instruments Q5000 system, equipped with a 25 position auto-sampler. Typically 1-5 mg of each sample was loaded onto a pre-tared aluminum pan and heated at 10° C./min from 25° C. to 350° C. A nitrogen purge at 25 mL/min was maintained over the sample throughout the measurement.

Some thermogravimetric analysis data were collected with a TA Discovery series TGA. A few milligrams of material were analyzed in an aluminum sample pan. The data was collected from room temperature to 300° C. with a 10° C. per minute scan rate.

### Formula I Zwitterion Form I

Formula I zwitterion Form I is an unsolvated form. It was first obtained by crystallization of Formula I from EtOH at pH 3.5-4.0.

XRPD analysis was conducted. FIG. 1 shows an XRPD pattern of the zwitterionic compound of Formula I Form I. XRPD peaks were identified and are included in Table 1 below.

TABLE 1

| No. | Pos. [°2Th.] | Rel. Int. [%] |
|---|---|---|
| 1 | 3.4 | 15 |
| 2 | 6.7 | 100 |
| 3 | 9.9 | 11 |
| 4 | 12.3 | 35 |
| 5 | 12.9 | 6 |
| 6 | 13.3 | 52 |
| 7 | 14.1 | 16 |
| 8 | 15.3 | 29 |
| 9 | 16.2 | 71 |
| 10 | 16.6 | 85 |
| 11 | 17.1 | 18 |
| 12 | 17.6 | 31 |
| 13 | 18.5 | 16 |
| 14 | 20.0 | 73 |
| 15 | 21.1 | 7 |
| 16 | 22.4 | 65 |
| 17 | 23.2 | 28 |
| 18 | 23.9 | 18 |
| 19 | 24.7 | 28 |
| 20 | 26.6 | 77 |
| 21 | 28.0 | 21 |
| 22 | 31.1 | 6 |

DSC analysis was performed. FIG. 2 shows a DSC thermogram of the zwitterionic compound of Formula I Form I. The DSC analysis revealed an endotherm with onset at about 265° C., followed by an exotherm corresponding to decomposition.

TGA analysis was performed. FIG. 3 shows a TGA thermogram of the zwitterionic compound of Formula I Form I. The TGA analysis showed that solids lost about 0.9% weight below about 150° C.

### Formula I Zwitterion Form II

Zwitterionic compound of Formula I Form II is an unsolvated form. It was obtained from crystallizations by dissolving Formula I zwitterion Form I in DMSO (4 volumes), followed by the addition of anti-solvent (40 volumes of IPAc, EtOAc, or MeCN) and stirring at room temperature. Formula I zwitterion Form II was also obtained after disproportionation of Formula I tromethamine salt in MeOH/water (0.4-0.6 water activity).

XRPD analysis was conducted. FIG. 4 shows an XRPD pattern of the zwitterionic compound of Formula I Form II. XRPD peaks were identified and are included in Table 2 below.

TABLE 2

| No. | Pos. [°2Th.] | Rel. Int. [%] |
|---|---|---|
| 1 | 7.3 | 51 |
| 2 | 12.5 | 8 |
| 3 | 13.6 | 6 |
| 4 | 14.5 | 36 |
| 5 | 15.7 | 14 |
| 6 | 17.0 | 5 |
| 7 | 17.5 | 18 |
| 8 | 18.1 | 73 |
| 9 | 20.2 | 19 |
| 10 | 21.1 | 21 |
| 11 | 21.7 | 22 |
| 12 | 22.6 | 10 |
| 13 | 23.2 | 7 |
| 14 | 24.1 | 100 |
| 15 | 25.0 | 34 |
| 16 | 25.4 | 34 |
| 17 | 26.0 | 8 |
| 18 | 27.6 | 11 |
| 19 | 28.2 | 5 |
| 20 | 29.1 | 33 |
| 21 | 29.4 | 16 |

DSC analysis was performed. FIG. 5 shows a DSC thermogram of the zwitterionic compound of Formula I Form II. As is shown, DSC afforded small endotherm with onset at about 135° C. and endotherm with onset at about 271° C., followed by exotherm corresponding to decomposition.

TGA analysis was performed. FIG. 6 shows a TGA thermogram of the zwitterionic compound of Formula I Form II. As indicated, solids lost about 0.7% weight below about 100° C. and about 1.1% weight at about 100-150° C.

### Formula I Zwitterion Hydrate

Formula I zwitterion hydrate is a crystalline form. It was obtained from the slurry of Formula I tromethamine salt in water due to disproportionation of the salt.

XRPD analysis was conducted. FIG. 7 shows an XRPD pattern of the zwitterionic compound of Formula I hydrate. XRPD peaks were identified and are included in Table 3 below.

TABLE 3

| No. | Pos. [°2Th.] | Rel. Int. [%] |
|---|---|---|
| 1 | 10.2 | 47.6 |
| 2 | 10.8 | 15.7 |
| 3 | 12.1 | 13.8 |
| 4 | 14.5 | 12.1 |
| 5 | 15.4 | 53.1 |
| 6 | 16.4 | 31.6 |
| 7 | 17.9 | 28.4 |
| 8 | 19.7 | 63.0 |
| 9 | 20.5 | 19.2 |
| 10 | 21.7 | 40.4 |
| 11 | 23.1 | 100.0 |
| 12 | 25.0 | 46.9 |
| 13 | 25.8 | 56.1 |
| 14 | 26.4 | 51.0 |
| 15 | 27.9 | 19.4 |
| 16 | 30.7 | 23.0 |
| 17 | 34.0 | 20.3 |

DSC analysis was performed. FIG. 8 shows a DSC thermogram of the zwitterionic compound of Formula I hydrate. As can be seen, DSC afforded broad endotherm with onset at about 46° C., small exotherm with onset at about 157° C. and melting endotherm with onset about 268° C., followed by exotherm corresponding to decomposition.

TGA analysis was performed. FIG. 9 shows a TGA thermogram of the zwitterionic compound of Formula I hydrate. As is shown, solids lost about 2.4% weight below about 100° C. and about 1.1% weight at about 100-250° C.

### Formula I Zwitterion Amorphous

Amorphous zwitterionic compound of Formula I was first obtained by ball milling Formula I (about 1.1 g) in stainless steel chamber containing stainless steel ball for about 5 min. Amorphous zwitterionic Formula I was also obtained using the following procedure: A reactor was charged with Formula I (1.0 equiv), water (20.0 V), and 50% NaOH (1.5 equiv), followed by stirring until dissolution was achieved. Then the reaction mixture was polish filtered, acidified with 1.0 M HCl (1.5 equiv), stirred for about 20 seconds, and filtered to isolate the resulting solids. Solids were washed with water (20V) and transferred to a nitrogen filled glove bag, followed by drying under nitrogen overnight.

XRPD analysis was conducted. FIG. 10 shows an XRPD pattern of the amorphous zwitterionic compound of Formula I.

### Formula I Tromethamine Salt Form I

Formula I tromethamine salt (tris salt) Form I was obtained by drying Formula I tromethamine salt ethanol solvate (at 0% RH and 25° C.). Formula I tromethamine salt Form I was also obtained by drying of Formula I tromethamine salt hydrate I under vacuum at 50° C. Formula I tromethamine salt Form I was also obtained from drying Formula I tromethamine salt methanol solvate under vacuum at 50° C.

FIG. 11 shows an XRPD pattern of Formula I tromethamine salt Form I. XRPD peaks were identified as noted in Table 4 below.

TABLE 4

| | Pos. | Rel. Int. |
|---|---|---|
| No. | [°2Th.] | [%] |
| 1 | 5.2 | 12 |
| 2 | 10.9 | 27 |
| 3 | 13.0 | 18 |
| 4 | 13.3 | 21 |
| 5 | 13.8 | 12 |
| 6 | 14.3 | 15 |
| 7 | 15.3 | 20 |
| 8 | 16.7 | 71 |
| 9 | 16.8 | 100 |
| 10 | 17.2 | 14 |
| 11 | 17.4 | 17 |
| 12 | 17.7 | 14 |
| 13 | 18.3 | 11 |
| 14 | 19.2 | 25 |
| 15 | 20.1 | 38 |
| 16 | 20.4 | 26 |
| 17 | 21.0 | 44 |
| 18 | 21.8 | 89 |
| 19 | 22.3 | 30 |
| 20 | 23.0 | 14 |
| 21 | 23.3 | 13 |
| 22 | 24.3 | 40 |
| 23 | 24.5 | 38 |
| 24 | 25.4 | 53 |
| 25 | 25.6 | 92 |
| 26 | 26.6 | 13 |
| 27 | 27.4 | 32 |
| 28 | 27.9 | 11 |
| 29 | 29.6 | 16 |
| 30 | 31.5 | 12 |
| 31 | 33.9 | 16 |

DSC analysis was performed. FIG. 12 shows a DSC thermogram of the Formula I tromethamine salt Form I. The melting onset is at about 129° C., followed by exotherm with onset at about 150° C. and decomposition.

TGA analysis was performed. FIG. 13 shows a TGA thermogram of the Formula I tromethamine salt Form I. As can be seen, the solids did not show any weight loss below about 150° C. prior to decomposition.

### Formula I Tromethamine Salt Form II

Formula I tromethamine salt Form II was obtained by dehydrating Formula I tromethamine salt hydrate II at about 80-120° C., as well as after dehydration at 0% RH at 25° C. Formula I tromethamine salt Form II was also obtained by vacuum drying Formula I methanol solvate I, Formula I methanol solvate II, or Formula I methanol solvate III at about 50-80° C. Formula I tromethamine salt Form II is unsolvated form, which rapidly absorbs moisture at about >20% RH and converts to Formula I tromethamine salt hydrate II.

XRPD analysis was conducted. FIG. 14 shows an XRPD pattern of Formula I tromethamine salt Form II. XRPD peaks were identified as noted in Table 5 below.

TABLE 5

| | Pos. | Rel. Int. |
|---|---|---|
| No. | [°2Th.] | [%] |
| 1 | 9.2 | 30 |
| 2 | 10.6 | 11 |
| 3 | 11.3 | 9 |
| 4 | 12.9 | 48 |
| 5 | 13.1 | 39 |
| 6 | 13.5 | 10 |
| 7 | 13.8 | 7 |
| 8 | 14.8 | 14 |
| 9 | 15.9 | 72 |
| 10 | 16.5 | 28 |
| 11 | 17.3 | 100 |
| 12 | 18.2 | 5 |
| 13 | 20.4 | 61 |
| 14 | 21.3 | 19 |
| 15 | 21.7 | 19 |
| 16 | 22.1 | 18 |
| 17 | 22.4 | 20 |
| 18 | 22.7 | 13 |
| 19 | 23.2 | 10 |
| 20 | 23.7 | 7 |
| 21 | 24.5 | 25 |
| 22 | 25.1 | 44 |
| 23 | 25.4 | 8 |
| 24 | 26.2 | 16 |
| 25 | 27.1 | 7 |
| 26 | 27.7 | 13 |
| 27 | 28.4 | 21 |

### Formula I Tromethamine Salt Hydrate I

Formula I tromethamine salt hydrate I was obtained by equilibrating of Formula I tromethamine salt ethanol solvate at ambient conditions and at about 98% RH at about 20-25° C. Formula I tromethamine salt hydrate I was also obtained by equilibrating of Formula I tromethamine salt methanol solvate at ambient conditions and at about 98% RH at about 20-25° C. Formula I tromethamine salt hydrate I was also obtained by equilibrating of Formula I tromethamine salt form I at about 80-98% RH at about 20-25° C.

XRPD analysis was conducted. FIG. 15 shows an XRPD pattern of the Formula I tromethamine salt hydrate I. XRPD peaks were identified and are included in Table 6 below.

TABLE 6

| | Pos. | Rel. Int. |
|---|---|---|
| No. | [°2Th.] | [%] |
| 1 | 5.9 | 13 |
| 2 | 7.1 | 12 |
| 3 | 9.8 | 13 |
| 4 | 12.7 | 19 |
| 5 | 13.9 | 67 |
| 6 | 14.1 | 20 |
| 7 | 14.5 | 16 |
| 8 | 15.2 | 14 |
| 9 | 16.0 | 11 |
| 10 | 17.4 | 39 |
| 11 | 17.8 | 21 |
| 12 | 18.7 | 13 |
| 13 | 20.0 | 47 |
| 14 | 20.3 | 62 |
| 15 | 20.7 | 11 |
| 16 | 21.9 | 37 |
| 17 | 22.7 | 35 |
| 18 | 23.3 | 100 |
| 19 | 24.2 | 31 |
| 20 | 24.9 | 23 |
| 21 | 25.3 | 53 |
| 22 | 26.3 | 16 |
| 23 | 26.7 | 16 |
| 24 | 27.2 | 23 |
| 25 | 27.8 | 38 |
| 26 | 28.5 | 31 |
| 27 | 29.1 | 33 |
| 28 | 30.6 | 26 |
| 29 | 32.3 | 15 |
| 30 | 35.5 | 22 |

DSC analysis was performed. FIG. 16 shows a DSC thermogram of the Formula I tromethamine salt hydrate I. The broad endotherm with onset at about 74° C. corresponds to the loss of water. The melting onset is at about 123° C., followed by exotherm with onset at about 145° C. and decomposition. After dehydration at about 100° C., Formula I tromethamine salt hydrate I converts to an unsolvated Formula I tromethamine salt Form I.

TGA analysis was performed. FIG. 17 shows a TGA thermogram of the Formula I tromethamine salt hydrate I. The solids lost about 6.2% weight below about 100° C., which corresponds to about 2.4 equivalents of water.

### Formula I Tromethamine Salt Hydrate II

Formula I tromethamine salt hydrate II was obtained by slurrying Formula I tromethamine salt ethanol solvate and Formula I tromethamine salt Form I in acetonitrile at ambient conditions by moisture adsorption from the air (at >10% RH). Formula I tromethamine salt hydrate II was also obtained by equilibrating of Formula I tromethamine salt form II at ambient conditions.

XRPD analysis was conducted. FIG. 18 shows an XRPD pattern of the Formula I tromethamine salt hydrate II. XRPD peaks were identified and are included in Table 7 below.

TABLE 7

| | Pos. | Rel. Int. |
|---|---|---|
| No. | [°2Th.] | [%] |
| 1 | 9.0 | 21 |
| 2 | 10.5 | 58 |
| 3 | 13.1 | 12 |
| 4 | 13.4 | 21 |
| 5 | 15.1 | 10 |
| 6 | 16.1 | 100 |
| 7 | 16.4 | 19 |
| 8 | 17.2 | 59 |
| 9 | 18.8 | 56 |
| 10 | 20.1 | 41 |
| 11 | 20.4 | 12 |
| 12 | 20.9 | 17 |
| 13 | 21.5 | 91 |
| 14 | 22.1 | 11 |
| 15 | 22.7 | 30 |
| 16 | 23.4 | 38 |
| 17 | 23.8 | 32 |
| 18 | 24.5 | 22 |
| 19 | 24.9 | 52 |
| 20 | 25.6 | 11 |
| 21 | 26.0 | 18 |
| 22 | 26.2 | 28 |
| 23 | 26.9 | 60 |
| 24 | 27.5 | 13 |
| 25 | 28.0 | 17 |
| 26 | 28.5 | 22 |

DSC analysis was performed. FIG. 19 shows a DSC thermogram of the Formula I tromethamine salt hydrate II. The broad endotherm with onset at about 48° C. corresponds to the loss of water. The melting onset is at about 130° C., followed by exotherm and decomposition. After dehydration at about 80° C., Formula I tromethamine salt hydrate II converts to an unsolvated form, Formula I tromethamine salt Form II.

TGA analysis was performed. FIG. 20 shows a TGA thermogram of the Formula I tromethamine salt hydrate II. The solids lost about 4.3% weight below about 100° C., which corresponds to about 1.8 equivalents of water.

### Formula I Tromethamine Salt Hydrate III

Formula I tromethamine salt hydrate III was obtained by charging a 20 mL vial with 220 mg of Formula I and about 1.1 eq Tris (50 mg) and 6 mL of MeCN/10% water. The slurry was stirred at about 50° C. overnight, followed by stirring at room temperature for about 3 days. Isolated solids were washed with 2 mL acetonitrile and dried under vacuum at about 40° C. overnight.

XRPD analysis was conducted. FIG. 21 shows an XRPD pattern of the Formula I tromethamine salt hydrate III. XRPD peaks were identified and are included in Table 8 below.

TABLE 8

| | Pos. | Rel. Int. |
|---|---|---|
| No. | [°2Th.] | [%] |
| 1 | 9.1 | 27 |
| 2 | 10.2 | 100 |
| 3 | 11.2 | 17 |
| 4 | 12.3 | 18 |
| 5 | 15.0 | 29 |
| 6 | 15.2 | 35 |
| 7 | 15.5 | 19 |
| 8 | 16.0 | 60 |
| 9 | 16.3 | 21 |
| 10 | 16.9 | 23 |
| 11 | 18.0 | 15 |
| 12 | 18.3 | 18 |
| 13 | 19.0 | 32 |
| 14 | 19.2 | 24 |
| 15 | 20.0 | 26 |
| 16 | 21.6 | 43 |
| 17 | 22.6 | 17 |
| 18 | 23.2 | 50 |
| 19 | 23.8 | 71 |
| 20 | 24.1 | 55 |
| 21 | 24.3 | 20 |
| 22 | 24.7 | 46 |
| 23 | 26.1 | 95 |
| 24 | 26.7 | 61 |
| 25 | 28.5 | 19 |
| 26 | 28.8 | 30 |

DSC analysis was performed. FIG. 22 shows a DSC thermogram of the Formula I tromethamine salt hydrate III. The broad endotherm at about 20-110° C. with onset at about 82° C. corresponds to the loss of water. The melting onset is at about 120° C., followed by exotherm with onset at about 154° C. and decomposition.

TGA analysis was performed. FIG. 23 shows a TGA thermogram of the Formula I tromethamine salt hydrate III. The solids lost about 4.8% weight below about 125° C., which corresponds to about 2 equivalents of water.

### Formula I Tromethamine Salt Hydrate IV

Formula I tromethamine salt hydrate IV was obtained by combining Formula I tromethamine salt Form I with acetone, DCM and toluene and equilibrating the solids obtained at ambient conditions.

XRPD analysis was conducted. FIG. 24 shows an XRPD pattern of the Formula I tromethamine salt hydrate IV. XRPD peaks were identified and are included in Table 9 below.

TABLE 9

| | Pos. | Rel. Int. |
|---|---|---|
| No. | [°2Th.] | [%] |
| 1 | 11.4 | 10 |
| 2 | 13.7 | 17 |
| 3 | 14.2 | 27 |
| 4 | 15.3 | 27 |
| 5 | 15.8 | 18 |
| 6 | 16.9 | 69 |
| 7 | 19.0 | 28 |
| 8 | 19.4 | 30 |
| 9 | 19.8 | 21 |
| 10 | 20.0 | 24 |
| 11 | 20.5 | 38 |
| 12 | 20.9 | 13 |
| 13 | 21.7 | 26 |
| 14 | 21.9 | 36 |
| 15 | 22.8 | 100 |
| 16 | 23.3 | 16 |
| 17 | 23.8 | 14 |
| 18 | 24.4 | 10 |
| 19 | 25.0 | 12 |
| 20 | 25.5 | 10 |
| 21 | 27.1 | 11 |
| 22 | 30.0 | 12 |
| 23 | 31.1 | 22 |
| 24 | 34.4 | 11 |

DSC analysis was performed. FIG. 25 shows a DSC thermogram of the Formula I tromethamine salt hydrate IV. The broad endotherm at about 20-90° C. with onset at about 48° C. corresponds to the loss of water, followed by small endotherm with onset at about 117° C. The melting onset is at about 148° C., followed by exotherm and decomposition.

TGA analysis was performed. FIG. 26 shows a TGA thermogram of the Formula I tromethamine salt hydrate IV. The solids lost about 2.6% weight below about 100° C., which corresponds to about 1 equivalent of water.

### Formula I Tromethamine Salt Methanol Solvate I

Formula I tromethamine salt methanol solvate I was obtained by charging a 100 mL jacketed reactor with 6 g of Formula I, about 1.1 equivalent Tris (1.36 g) and 60 mL of MeOH. The slurry was stirred at about 50° C. overnight to afford very large particles of MeOH solvate I, which was confirmed by single crystal x-ray analysis. Single crystal x-ray analysis showed two molecules of methanol per one molecule of Formula I tromethamine salt. Formula I tromethamine salt methanol solvate I loses methanol very rapidly at ambient conditions and converts to tromethamine salt methanol solvate III, which further converts to tromethamine salt hydrate I. Formula I tromethamine salt methanol solvate I also converted to methanol solvate II in THF and MTBE slurries. Formula I tromethamine salt methanol solvate I was also obtained by equilibration of Formula I tromethamine salt form I, Formula I tromethamine salt hydrate I, Formula I tromethamine salt hydrate II, Formula I tromethamine salt hydrate III, Formula I tromethamine salt hydrate IV, Formula I tromethamine salt methanol solvate II, Formula I tromethamine salt methanol solvate III, Formula I tromethamine salt ethanol solvate, and Formula I tromethamine salt MTBE solvate in up to 10% water/MeOH mixtures at ambient conditions.

XRPD analysis was conducted. FIG. 27 shows an XRPD pattern of the Formula I tromethamine salt methanol solvate I. XRPD peaks were identified and are included in Table 10 below.

TABLE 10

| | Pos. | Rel. Int. |
|---|---|---|
| No. | [°2Th.] | [%] |
| 1 | 7.0 | 10 |
| 2 | 10.8 | 100 |
| 3 | 13.0 | 44 |
| 4 | 13.3 | 93 |
| 5 | 13.9 | 16 |
| 6 | 14.5 | 31 |
| 7 | 19.5 | 15 |
| 8 | 20.0 | 50 |
| 9 | 21.4 | 49 |
| 10 | 22.3 | 73 |
| 11 | 22.9 | 14 |
| 12 | 24.1 | 14 |
| 13 | 27.6 | 12 |
| 14 | 30.1 | 13 |
| 15 | 30.7 | 10 |
| 16 | 32.2 | 19 |
| 17 | 32.7 | 16 |

### Formula I Tromethamine Salt Methanol Solvate II

Formula I tromethamine salt methanol solvate II was obtained from the slurry of Formula I tromethamine MeOH solvate I in THE, as described above. It was also obtained from the Formula I tromethamine salt formation in MeOH/MTBE and MeOH/5% water/MTBE.

XRPD analysis was conducted. FIG. 28 shows an XRPD pattern of the Formula I tromethamine salt methanol solvate II. XRPD peaks were identified and are included in Table 11 below.

TABLE 11

| | Pos. | Rel. Int. |
|---|---|---|
| No. | [°2Th.] | [%] |
| 1 | 5.4 | 40 |
| 2 | 6.9 | 21 |
| 3 | 10.7 | 94 |
| 4 | 12.3 | 21 |
| 5 | 12.9 | 100 |
| 6 | 13.7 | 34 |
| 7 | 14.3 | 48 |
| 8 | 15.9 | 23 |
| 9 | 16.8 | 19 |
| 10 | 17.4 | 17 |
| 11 | 17.8 | 24 |
| 12 | 18.7 | 19 |
| 13 | 19.0 | 14 |
| 14 | 20.3 | 27 |
| 15 | 21.2 | 62 |
| 16 | 21.6 | 69 |
| 17 | 22.3 | 36 |
| 18 | 22.7 | 62 |
| 19 | 23.2 | 27 |
| 20 | 23.7 | 27 |
| 21 | 24.0 | 22 |
| 22 | 24.8 | 15 |
| 23 | 25.3 | 17 |
| 24 | 26.2 | 30 |
| 25 | 26.6 | 50 |
| 26 | 28.4 | 16 |
| 27 | 28.9 | 16 |
| 28 | 30.4 | 15 |

### Formula I Tromethamine Salt Methanol Solvate III

Formula I tromethamine salt methanol solvate III was obtained by equilibrating Formula I tromethamine salt methanol solvate I for about 5-10 minutes at ambient conditions. It was also obtained by equilibrating Formula I tromethamine salt methanol solvate II for about 5-10 min at ambient conditions. Tromethamine methanol solvate III fully converted to Formula I tromethamine salt hydrate I at ambient conditions within about 1 hour (depending on % RH).

XRPD analysis was conducted. FIG. 29 shows an XRPD pattern of the Formula I tromethamine salt methanol solvate III. XRPD peaks were identified and are included in Table 12 below.

TABLE 12

| | Pos. | Rel. Int. |
|---|---|---|
| No. | [°2Th.] | [%] |
| 1 | 5.6 | 11 |
| 2 | 7.1 | 13 |
| 3 | 12.6 | 33 |
| 4 | 13.3 | 35 |
| 5 | 13.8 | 84 |
| 6 | 14.3 | 26 |
| 7 | 15.1 | 19 |
| 8 | 15.9 | 25 |
| 9 | 17.4 | 83 |
| 10 | 17.7 | 23 |
| 11 | 19.2 | 30 |
| 12 | 19.9 | 47 |
| 13 | 20.2 | 95 |
| 14 | 21.8 | 33 |
| 15 | 22.6 | 56 |
| 16 | 23.2 | 100 |
| 17 | 24.2 | 75 |
| 18 | 24.8 | 52 |
| 19 | 25.2 | 73 |
| 20 | 26.1 | 16 |
| 21 | 26.7 | 28 |
| 22 | 27.1 | 30 |
| 23 | 27.6 | 49 |
| 24 | 28.4 | 52 |
| 25 | 29.1 | 58 |
| 26 | 30.4 | 26 |
| 27 | 30.9 | 13 |
| 28 | 32.0 | 17 |
| 29 | 32.7 | 19 |
| 30 | 35.4 | 27 |

DSC analysis was performed. FIG. 30 shows a DSC thermogram of the Formula I tromethamine salt methanol solvate III. Two endotherms with onsets at about 80 and 110° C. were observed. These may correspond to the loss of methanol and water. The melting onset is at about 127° C., followed by exotherm with onset at about 145° C. and decomposition. After the desolvation, Formula I tromethamine methanol solvate III converted to an unsolvated form-Formula I tromethamine salt Form I.

TGA analysis was performed. FIG. 31 shows a TGA thermogram of the Formula I tromethamine salt methanol solvate III. The solids lost about 6.0% weight below about 120° C. Karl Fischer (KF) analysis showed about 1.1% water. ¹H NMR analysis was performed, and the resultant spectrum showed about 1.1 equivalent of methanol which corresponds to about 4.7 wt %.

### Formula I Tromethamine Salt MTBE Solvate

Formula I tromethamine salt MTBE solvate was obtained by slurrying Formula I tromethamine salt Form I (50 mg) in MTBE (1 mL) at 50° C. The wet solids were analyzed by XRPD. The MTBE solvate converted to disordered material at ambient conditions and after vacuum drying at 50° C.

XRPD analysis was conducted. FIG. 32 shows an XRPD pattern of the Formula I tromethamine salt MTBE solvate. XRPD peaks were identified and are included in Table 13 below.

TABLE 13

| | Pos. | Rel. Int. |
|---|---|---|
| No. | [°2Th.] | [%] |
| 1 | 5.8 | 61 |
| 2 | 11.4 | 37 |
| 3 | 11.8 | 14 |
| 4 | 12.7 | 30 |
| 5 | 14.5 | 25 |
| 6 | 15.6 | 30 |
| 7 | 16.9 | 10 |
| 8 | 18.1 | 34 |
| 9 | 18.4 | 40 |
| 10 | 19.0 | 44 |
| 11 | 19.7 | 36 |
| 12 | 20.4 | 100 |
| 13 | 21.1 | 29 |
| 14 | 22.1 | 35 |
| 15 | 23.4 | 41 |
| 16 | 24.7 | 37 |
| 17 | 25.9 | 38 |
| 18 | 26.7 | 16 |
| 19 | 29.3 | 11 |
| 20 | 30.3 | 13 |

DSC analysis was performed. FIG. 33 shows a DSC thermogram of the Formula I tromethamine salt MTBE solvate. The broad endotherm with onset at about 85° C. corresponds to the loss of solvent. The melting onset is at about 147° C., followed by exotherm and decomposition.

TGA analysis was performed. FIG. 34 shows a TGA thermogram of the Formula I tromethamine salt MTBE solvate. The solids showed about 6.2% weight loss below about 120° C. corresponding to about 0.5 equivalents of MTBE.

### Formula I Tromethamine Salt Amorphous

Formula I tromethamine salt amorphous form was obtained by Formula I tromethamine salt (about 0.89 mg) in about 5 mL of THF at high temperature (about 65° C.), followed by fast evaporation on rotary evaporator at about 40° C. (bath temperature) to obtain foam, and then drying under vacuum at about 50° C. for about 2 hours.

XRPD analysis was conducted. FIG. 35 shows an XRPD pattern of the Formula I tromethamine salt amorphous form.

### Formula I Tromethamine Salt Ethanol Solvate

Formula I tromethamine salt ethanol solvate was obtained by charging a 4 mL vial with 52.5 mg of zwitterionic Formula I, about 1.1 equivalent Tris (12 mg) and 1 mL of ethanol. The slurry was stirred at about 50° C. for about 5 hours and at room temperature overnight. The sample of wet solids was analyzed by XRPD and afforded unique XRPD pattern of ethanol solvate, which converted to Formula I tromethamine salt hydrate I at ambient conditions.

XRPD analysis was conducted. FIG. 36 shows an XRPD pattern of the Formula I tromethamine salt ethanol solvate. XRPD peaks were obtained and are depicted in Table 14 below.

TABLE 14

| | Pos. | Rel. Int. |
|---|---|---|
| No. | [°2Th.] | [%] |
| 1 | 5.3 | 53 |
| 2 | 10.7 | 34 |
| 3 | 12.1 | 52 |
| 4 | 12.9 | 93 |
| 5 | 13.7 | 39 |
| 6 | 14.3 | 39 |
| 7 | 15.1 | 22 |
| 8 | 15.8 | 22 |
| 9 | 16.6 | 21 |
| 10 | 16.9 | 21 |
| 11 | 18.0 | 31 |
| 12 | 18.8 | 40 |
| 13 | 20.4 | 22 |
| 14 | 21.0 | 83 |
| 15 | 21.6 | 100 |
| 16 | 22.3 | 97 |
| 17 | 22.6 | 67 |
| 18 | 23.2 | 22 |
| 19 | 23.8 | 56 |
| 20 | 24.7 | 22 |
| 21 | 25.3 | 23 |
| 22 | 26.1 | 79 |
| 23 | 26.3 | 88 |
| 24 | 26.7 | 31 |
| 25 | 27.0 | 24 |
| 26 | 27.8 | 31 |
| 27 | 28.2 | 21 |
| 28 | 28.7 | 19 |
| 29 | 29.9 | 34 |
| 30 | 36.9 | 21 |

Formula I p-TSA Salt Form I

Formula I p-TSA salt Form I was obtained by dissolving 52.7 mg Formula I Form I with 1.1 eq. of p-toluenesulfonic acid monohydrate (18.8 mg) in 1 mL EtOH at 50° C., followed by cooling to room temperature and stirring at this temperature for about 16 hours. Formula I p-TSA salt Form I was also obtained using a similar procedure in following solvents: THF, MeTHF, MeCN, IPAc, IPA, EtOAc. Formula I p-TSA salt Form I was also made from Formula I Form I in MeCN/water or EtOH/water mixtures (9:1 or 8:2) with NaOH (1.1 equiv) and p-toluenesulfonic acid monohydrate (2.2 equiv) or from Formula I tromethamine salt Form I in MeCN/water or EtOH/water mixtures (9:1 or 8:2) with NaOH (1.1 equiv) and p-toluenesulfonic acid monohydrate (3.2 equiv). Formula I p-TSA salt Form I was also prepared by slurrying Formula I p-TSA salt hydrate in MeCN at 20, 35, 55 and 70° C.

XRPD analysis was conducted. FIG. 37 shows an XRPD pattern of the Formula I p-TSA salt Form I. XRPD peaks were identified and are included in Table 15 below.

TABLE 15

| | Pos. | Rel. Int. |
|---|---|---|
| No. | [°2Th.] | [%] |
| 1 | 3.0 | 38.3 |
| 2 | 9.0 | 14.1 |
| 3 | 11.8 | 23.2 |
| 4 | 12.0 | 29.4 |
| 5 | 13.1 | 28.6 |
| 6 | 15.0 | 32.9 |
| 7 | 16.6 | 100.0 |
| 8 | 17.5 | 55.0 |
| 9 | 18.7 | 21.0 |
| 10 | 20.9 | 78.8 |
| 11 | 21.5 | 54.7 |
| 12 | 22.3 | 86.5 |
| 13 | 23.4 | 91.1 |
| 14 | 23.5 | 78.6 |
| 15 | 23.7 | 43.1 |
| 16 | 24.0 | 19.8 |
| 17 | 24.3 | 28.9 |
| 18 | 25.0 | 28.1 |
| 19 | 26.0 | 49.7 |
| 20 | 26.4 | 25.3 |
| 21 | 26.7 | 23.1 |
| 22 | 28.0 | 38.6 |
| 23 | 28.9 | 18.3 |
| 24 | 33.7 | 18.2 |

DSC analysis was performed. FIG. 38 shows a DSC thermogram of the Formula I p-TSA salt Form I. DSC afforded melting endotherm with onset at about 198° C., followed by exotherm with onset at about 208° C. and decomposition.

TGA analysis was performed. FIG. 39 shows a TGA thermogram of the Formula I p-TSA salt Form I. The solids lost about 0.6% weight below about 170° C. and about 3.0% weight at about 170-235° C., which corresponds to the loss of water due to decomposition

Formula I p-TSA Salt Form II

Formula I p-TSA salt Form II was obtained by stirring of Formula I p-TSA salt Form I for 3 weeks at room temperature in the following solvents: MeCN, MeOH, EtOH, IPA, acetone, MEK, MIBK, THF, MeTHF, EtOAc, IPAc, MTBE, toluene, and heptane

XRPD analysis was conducted. FIG. 40 shows an XRPD pattern of the Formula I p-TSA salt Form II. XRPD peaks were identified and are included in Table 16 below.

TABLE 16

| | Pos. | Rel. Int. |
|---|---|---|
| No. | [°2Th.] | [%] |
| 1 | 3.0 | 41.3 |
| 2 | 11.8 | 49.8 |
| 3 | 14.8 | 61.7 |
| 4 | 16.5 | 51.4 |
| 5 | 16.8 | 54.6 |
| 6 | 17.5 | 40.8 |
| 7 | 18.7 | 20.0 |
| 8 | 20.8 | 66.7 |
| 9 | 21.6 | 41.3 |
| 10 | 22.1 | 54.1 |
| 11 | 22.4 | 19.3 |
| 12 | 23.2 | 100.0 |
| 13 | 23.8 | 70.7 |
| 14 | 24.4 | 23.4 |
| 15 | 26.1 | 17.8 |
| 16 | 26.5 | 18.2 |
| 17 | 26.8 | 29.6 |
| 18 | 27.9 | 19.3 |
| 19 | 29.0 | 24.8 |

DSC analysis was performed. FIG. 41 shows a DSC thermogram of the Formula I p-TSA salt Form II. DSC afforded broad exotherm with onset at about 155° C., melting endotherm with onset at about 202° C., followed by exotherm with onset at about 209° C. and decomposition.

TGA analysis was performed. FIG. 42 shows a TGA thermogram of the Formula I p-TSA salt Form II. The solids lost about 0.7% weight below about 175° C. and about 3.0% weight at about 175-238° C., which corresponds to the loss of water due to decomposition.

Formula I p-TSA salt Form III

Formula I p-TSA salt Form III was obtained by stirring of Formula I p-TSA salt Form I obtained after stirring of Formula I p-TSA salt Form I for about 3 weeks at room temperature in the following solvents: EtOAc, toluene, and in MeCN/water (with 0.6, 0.7, and 0.95 water activity).

XRPD analysis was conducted. FIG. 43 shows an XRPD pattern of the Formula I p-TSA salt Form III. XRPD peaks were identified and are included in Table 17 below.

TABLE 17

| | Pos. | Rel. Int. |
|---|---|---|
| No. | [°2Th.] | [%] |
| 1 | 11.9 | 22.6 |
| 2 | 12.3 | 19.0 |
| 3 | 13.5 | 15.1 |
| 4 | 14.8 | 46.5 |
| 5 | 15.0 | 18.2 |
| 6 | 16.6 | 48.8 |
| 7 | 16.9 | 67.8 |
| 8 | 17.6 | 54.6 |
| 9 | 18.8 | 24.0 |
| 10 | 20.8 | 51.4 |
| 11 | 21.6 | 32.0 |
| 12 | 22.1 | 55.5 |
| 13 | 22.5 | 26.7 |
| 14 | 23.2 | 100.0 |
| 15 | 23.7 | 34.8 |
| 16 | 24.0 | 31.1 |
| 17 | 24.6 | 32.7 |
| 18 | 26.2 | 21.6 |
| 19 | 26.6 | 28.8 |
| 20 | 28.0 | 26.4 |
| 21 | 29.1 | 24.8 |
| 22 | 34.0 | 16.0 |
| 23 | 35.7 | 19.2 |

DSC analysis was performed. FIG. 44 shows a DSC thermogram of the Formula I p-TSA salt Form III. DSC afforded broad exotherm with onset at about 156° C., melting endotherm with onset at about 205° C., followed by immediate exotherm and decomposition.

TGA analysis was performed. FIG. 45 shows a TGA thermogram of the Formula I p-TSA salt Form III. The solids lost about 0.5% weight below about 175° C. and about 2.5% weight at about 175-235° C., which corresponds to the loss of water due to decomposition

Formula I p-TSA Salt Hydrate

Formula I p-TSA salt hydrate was obtained by stirring Formula I tromethamine salt in water with 2 equivalents of p-toluenesulfonic acid monohydrate at 50° C. and at room temperature. Formula I p-TSA salt hydrate was also obtained from competition slurries of Formula I p-TSA salt Form I and hydrate in MeCN/water mixtures at 70° C. with ≥0.4 water activity. Formula I p-TSA salt hydrate was also prepared from Formula I Form I with or without p-TSA salt hydrate seeds in MeCN/water mixtures (9:1 or 8:2) with NaOH (1.1 equiv) and p-toluenesulfonic acid monohydrate (2.2 equiv) or from Formula I tromethamine salt Form I in MeCN/water mixtures (9:1 or 8:2) with NaOH (1.1 equiv) and p-toluenesulfonic acid monohydrate (3.2 equiv).

XRPD analysis was conducted. FIG. 46 shows an XRPD pattern of the Formula I p-TSA salt hydrate. XRPD peaks were identified and are included in Table 18 below.

TABLE 18

| | Pos. | Rel. Int. |
|---|---|---|
| No. | [°2Th.] | [%] |
| 1 | 6.6 | 43.5 |
| 2 | 8.6 | 23.4 |
| 3 | 11.3 | 19.7 |
| 4 | 13.3 | 26.0 |
| 5 | 15.6 | 24.0 |
| 6 | 17.1 | 43.2 |
| 7 | 17.7 | 32.0 |
| 8 | 18.4 | 20.2 |
| 9 | 19.8 | 100.0 |
| 10 | 20.7 | 29.4 |
| 11 | 21.7 | 22.4 |
| 12 | 22.5 | 85.4 |
| 13 | 23.7 | 31.1 |
| 14 | 23.9 | 20.9 |
| 15 | 24.7 | 19.9 |
| 16 | 24.9 | 28.6 |
| 17 | 25.5 | 65.0 |
| 18 | 25.7 | 57.9 |
| 19 | 26.9 | 46.7 |
| 20 | 28.4 | 93.0 |
| 21 | 29.7 | 26.9 |
| 22 | 31.7 | 20.8 |
| 23 | 32.0 | 19.0 |
| 24 | 33.1 | 20.2 |

DSC analysis was performed. FIG. 47 shows a DSC thermogram of the Formula I p-TSA salt hydrate. DSC afforded broad endotherm with onset at about 144° C., followed by broad melting endotherm with onset at about 192° C., exotherm with onset at about 214° C., and decomposition.

TGA analysis was performed. FIG. 48 shows a TGA thermogram of the Formula I p-TSA salt hydrate. The solids lost about 3.6% weight below about 166° C. and about 2.4% weight at about 166-236° C., which corresponds to the loss of water due to decomposition.

Unless otherwise defined, all technical and scientific terms used herein have the same meaning as commonly understood by one of ordinary skill in the art to which this disclosure belongs.

Thus, it should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1.-133. (canceled)

134. A method of treating or preventing a disease or condition in a patient in need thereof, comprising administering a therapeutically effective amount of an FXR agonist, wherein the disease or condition is congenital hepatic fibrosis, and wherein the FXR agonist comprises a solid form of a compound of the following formula: 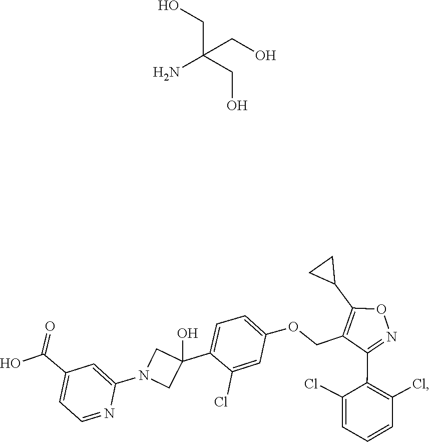 [SMILES: NC(CO)(CO)CO ; O=C(O)c1ccnc(N2CC(O)(c3ccc(OCc4c(-c5c(Cl)cccc5Cl)noc4C4CC4)cc3Cl)C2)c1]
wherein the solid form is tromethamine salt Form I characterized by an X-ray diffraction pattern having 2θ-reflections at 5.2, 16.8, and 25.6 degrees 2θ, plus or minus 0.2 degrees 2θ;
wherein the solid form is tromethamine salt Form II characterized by an X-ray diffraction pattern having 2θ-reflections at 9.2, 15.9, and 17.3 degrees 2θ, plus or minus 0.2 degrees 2θ;
wherein the solid form is tromethamine salt hydrate I characterized by an X-ray diffraction pattern having 2θ-reflections at 5.9, 13.9, 23.3 degrees 2θ, plus or minus 0.2 degrees 2θ;
wherein the solid form is tromethamine salt hydrate II characterized by an X-ray diffraction pattern having 2θ-reflections at 10.5, 16.1, and 21.5 degrees 2θ, plus or minus 0.2 degrees 2θ;
wherein the solid form is tromethamine salt hydrate III characterized by an X-ray diffraction pattern having 2θ-reflections at 10.2, 16.0, and 23.2 degrees 2θ, plus or minus 0.2 degrees 2θ;
wherein the solid form is tromethamine salt hydrate IV characterized by an X-ray diffraction pattern having 2θ-reflections at 11.4, 16.9, and 22.8 degrees 2θ, plus or minus 0.2 degrees 2θ;
wherein the solid form is tromethamine salt methanol solvate I characterized by an X-ray diffraction pattern having 2θ-reflections at 10.8, 13.3, and 22.3 degrees 2θ, plus or minus 0.2 degrees 2θ;
wherein the solid form is tromethamine salt methanol solvate II characterized by an X-ray diffraction pattern having 2θ-reflections at 5.4, 12.9, and 22.7 degrees 2θ, plus or minus 0.2 degrees 2θ;
wherein the solid form is tromethamine salt methanol solvate III characterized by an X-ray diffraction pattern having 2θ-reflections at 12.6, 13.3, and 13.8 degrees 2θ, plus or minus 0.2 degrees 2θ;
wherein the solid form is MTBE solvate characterized by an X-ray diffraction pattern having 2θ-reflections at 5.8, 11.4, 15.6, and 20.4 degrees 2θ, plus or minus 0.2 degrees 2θ; or
wherein the solid form is tromethamine salt amorphous having an X-ray diffraction pattern substantially as shown in FIG. 35.

135. (canceled)

136. The method of claim 134, wherein the tromethamine salt Form I is characterized by an X-ray diffraction pattern further comprising 2θ-reflections at 10.9, 15.3, and 21.8 degrees 2θ, plus or minus 0.2 degrees 2θ.

137. The method of claim 134, wherein the tromethamine salt Form I characterized by an X-ray diffraction pattern further comprising 2θ-reflections at 13.3, 20.1, 20.4, 21.0, and 24.3 degrees 2θ, plus or minus 0.2 degrees 2θ.

138. The method of claim 134, wherein the tromethamine salt Form I has a differential scanning calorimetry thermogram comprising an endotherm with onset at about 129° C.

139. The method of claim 134, wherein the tromethamine salt Form II is characterized by an X-ray diffraction pattern further comprising 2θ-reflections at 12.9, 20.4, 24.5, and 25.1 degrees 2θ, plus or minus 0.2 degrees 2θ.

140. The method of claim 134, wherein the tromethamine salt hydrate I is characterized by an X-ray diffraction pattern further comprising 2θ-reflections at 7.1, 12.7, and 20.3 degrees 2θ, plus or minus 0.2 degrees 2θ.

141. The method of claim 134, wherein the tromethamine salt hydrate I is characterized by an X-ray diffraction pattern further comprising 2θ-reflections at 9.8, 17.4, 22.7, and 25.3 degrees 2θ, plus or minus 0.2 degrees 2θ.

142. The method of claim 134, wherein the tromethamine salt hydrate I has a differential scanning calorimetry thermogram comprising an endotherm with onset at about 74° C., an endotherm with onset at about 123° C., and an exotherm with onset at about 145° C.

143. The method of claim 134, wherein the tromethamine salt hydrate II is characterized by an X-ray diffraction pattern further comprising 2θ-reflections at 9.0, 17.2, and 18.8 degrees 2θ, plus or minus 0.2 degrees 2θ.

144. The method of claim 134, wherein the tromethamine salt hydrate II is characterized by an X-ray diffraction pattern further comprising 2θ-reflections at 13.4, 20.1, 24.9, and 26.9 degrees 2θ, plus or minus 0.2 degrees 2θ.

145. The method of claim 134, wherein the tromethamine salt hydrate II has a differential scanning calorimetry thermogram comprising an endotherm with onset at about 48° C. and an endotherm with onset at about 130° C.

146. The method of claim 134, wherein the tromethamine salt hydrate III is characterized by an X-ray diffraction pattern further comprising 2θ-reflections at 9.1, 15.2, and 23.8 degrees 2θ, plus or minus 0.2 degrees 2θ.

147. The method of claim 134, wherein the tromethamine salt hydrate III is characterized by an X-ray diffraction pattern further comprising 2θ-reflections at 11.2, 12.3, 19.0, 21.6, and 26.1 degrees 2θ, plus or minus 0.2 degrees 2θ.

148. The method of claim 134, wherein the tromethamine salt hydrate III has a differential scanning calorimetry thermogram comprising an endotherm with onset at about 82° C., an endotherm with onset at about 120° C., and an exotherm with onset at about 154° C.

149. The method of claim 134, wherein the tromethamine salt hydrate IV is characterized by an X-ray diffraction pattern further comprising 2θ-reflections at 14.2, 15.3, and 20.5 degrees 2θ, plus or minus 0.2 degrees 2θ.

150. The method of claim 134, wherein the tromethamine salt hydrate IV is characterized by an X-ray diffraction pattern further comprising 2θ-reflections at 13.7, 15.8, 19.4, and 21.9 degrees 2θ, plus or minus 0.2 degrees 2θ.

151. The method of claim 134, wherein the tromethamine salt hydrate IV has a differential scanning calorimetry thermogram comprising an endotherm with onset at about 48° C., an endotherm with onset at about 117° C. and an endotherm with onset at about 148° C.

152. The method of claim 134, wherein the tromethamine salt methanol solvate I is characterized by an X-ray diffraction pattern further comprising 2θ-reflections at 14.5, 20.0, and 21.4 degrees 2θ, plus or minus 0.2 degrees 2θ.

153. The method of claim 134, wherein the tromethamine salt methanol solvate II is characterized by an X-ray diffraction pattern further comprising 2θ-reflections at 10.7, 14.3, and 15.9 degrees 2θ, plus or minus 0.2 degrees 2θ.

154. The method of claim 134, wherein the tromethamine salt methanol solvate II is characterized by an X-ray diffraction pattern further comprising 2θ-reflections at 21.2, and 26.6 degrees 2θ, plus or minus 0.2 degrees 2θ.

155. The method of claim 134, wherein the tromethamine salt methanol solvate III is characterized by an X-ray diffraction pattern further comprising 2θ-reflections at 14.3, 17.4, and 23.2 degrees 2θ, plus or minus 0.2 degrees 2θ.

156. The method of claim 134, wherein the tromethamine salt methanol solvate III is characterized by an X-ray diffraction pattern further comprising 2θ-reflections at 20.2, 24.2, 25.2, and 29.1 degrees 2θ, plus or minus 0.2 degrees 2θ.

157. The method of claim 134, wherein the tromethamine salt methanol solvate III has a differential scanning calorimetry thermogram comprising an endotherm with onset at about 80° C., an endotherm with onset at about 110° C., an endotherm with onset at about 127° C., and an exotherm with onset at about 145° C.

158. The method of claim 134, wherein the MTBE solvate is characterized by an X-ray diffraction pattern further comprising 2θ-reflections at 14.5, 18.4, 23.4, and 25.9 degrees 2θ, plus or minus 0.2 degrees 2θ.

159. The method of claim 134, wherein the MTBE solvate is characterized by an X-ray diffraction pattern further comprising 2θ-reflections at 12.7, 19.0, and 24.7 degrees 2θ, plus or minus 0.2 degrees 2θ.

160. The method of claim 134, wherein the MTBE solvate has a differential scanning calorimetry thermogram comprising an endotherm with onset at about 85° C. and an endotherm with onset at about 147° C.

161. A pharmaceutical composition comprising a therapeutically effective amount of a solid form of a compound of the following formula: 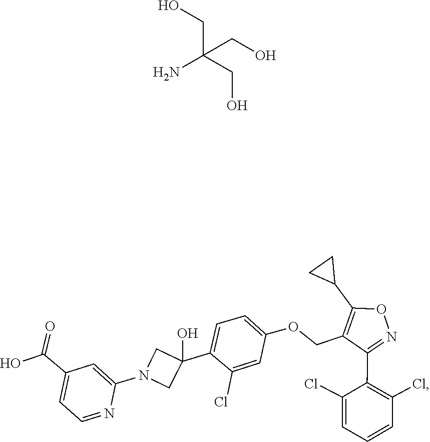 [SMILES: NC(CO)(CO)CO ; O=C(O)c1ccnc(N2CC(O)(c3ccc(OCc4c(-c5c(Cl)cccc5Cl)noc4C4CC4)cc3Cl)C2)c1]
wherein the solid form is tromethamine salt Form I characterized by an X-ray diffraction pattern having 2θ-reflections at 5.2, 16.8, and 25.6 degrees 2θ, plus or minus 0.2 degrees 2θ;
wherein the solid form is tromethamine salt Form II characterized by an X-ray diffraction pattern having 2θ-reflections at 9.2, 15.9, and 17.3 degrees 2θ, plus or minus 0.2 degrees 2θ;
wherein the solid form is tromethamine salt hydrate I characterized by an X-ray diffraction pattern having 2θ-reflections at 5.9, 13.9, 23.3 degrees 2θ, plus or minus 0.2 degrees 2θ;
wherein the solid form is tromethamine salt hydrate II characterized by an X-ray diffraction pattern having 2θ-reflections at 10.5, 16.1, and 21.5 degrees 2θ, plus or minus 0.2 degrees 2θ;
wherein the solid form is tromethamine salt hydrate III characterized by an X-ray diffraction pattern having 2θ-reflections at 10.2, 16.0, and 23.2 degrees 2θ, plus or minus 0.2 degrees 2θ;
wherein the solid form is tromethamine salt hydrate IV characterized by an X-ray diffraction pattern having 2θ-reflections at 11.4, 16.9, and 22.8 degrees 2θ, plus or minus 0.2 degrees 2θ;
wherein the solid form is tromethamine salt methanol solvate I characterized by an X-ray diffraction pattern having 2θ-reflections at 10.8, 13.3, and 22.3 degrees 2θ, plus or minus 0.2 degrees 2θ;
wherein the solid form is tromethamine salt methanol solvate II characterized by an X-ray diffraction pattern having 2θ-reflections at 5.4, 12.9, and 22.7 degrees 2θ, plus or minus 0.2 degrees 2θ;
wherein the solid form is tromethamine salt methanol solvate III characterized by an X-ray diffraction pattern having 2θ-reflections at 12.6, 13.3, and 13.8 degrees 2θ, plus or minus 0.2 degrees 2θ;
wherein the solid form is MTBE solvate characterized by an X-ray diffraction pattern having 2θ-reflections at 5.8, 11.4, 15.6, and 20.4 degrees 2θ, plus or minus 0.2 degrees 2θ; or
wherein the solid form is tromethamine salt amorphous having an X-ray diffraction pattern substantially as shown in FIG. 35.

162. The pharmaceutical composition of claim 161, further comprising one to three additional therapeutic agents, wherein at least one of the additional therapeutic agents is active against a liver disease.
